(19) United States
(12) Patent Application Publication
(10) Pub. No.: US 2026/0078079 A1
(43) Pub. Date: Mar. 19, 2026

(54) FRAGRANCE AND FLAVOR COMPOSITIONS COMPRISING NORBORNANE AND NORBORNENE DERIVATIVES

(71) Applicant: OSMO LABS, PBC, New York, NY (US)

(72) Inventors: Bradley B. GILBERT, Milford, NJ (US); Benjamin AMORELLI, Brielle, NJ (US); Gregory L. WARREN, Winchester, MA (US); Alex J. BERKOWITZ, Brooklyn, NY (US); Andrew P. PATRON, Oceanside, CA (US)

(21) Appl. No.: 19/330,779

(22) Filed: Sep. 16, 2025

Related U.S. Application Data
(60) Provisional application No. 63/695,339, filed on Sep. 16, 2024.

(51) Int. Cl. C07C 69/608; A23L 27/20; C07C 33/14; C07C 43/162; C07C 49/21; C07C 255/31; C07D 303/12; C07D 307/46; C11B 9/00

(52) U.S. Cl. CPC C07C 69/608; A23L 27/204; A23L 27/2052; C07C 33/14; C07C 43/162; C07C 49/21; C07C 255/31; C07D 303/12; C07D 307/46; C11B 9/0034; C11B 9/0076; C07C 2601/16

(57) ABSTRACT

The present application relates to bicyclo[2.2.1]heptane and bicyclo[2.2.1]heptene derivatives, methods of making them, and methods of using them as flavor and fragrance ingredients in food, cosmetic, pharmaceutical, consumer, and other compositions and products.

### CROSS-REFERENCE TO RELATED APPLICATIONS

This application claims priority to, and the benefit of, U.S. Provisional Application No. 63/695,339, filed on Sep. 16, 2024, the contents of which are incorporated by reference in its entirety.

### FIELD OF THE APPLICATION

The present application relates to bicyclo[2.2.1]heptane and bicyclo[2.2.1]heptene derivatives, methods of making them, and methods of using them as flavor and fragrance ingredients in food, cosmetic, pharmaceutical, consumer, and other compositions and products.

### BACKGROUND

Scent is an important factor used to produce a sense of anticipation, quality, palatability, and security to many consumer products. Flavor is particularly important for foodstuffs. Identifying effective aromas and flavors to impart in a product is an element that contributes to the success of the product, and is useful in product marketing, consumer satisfaction, and consumer retention. Toasted coconut and other nutty smells may be particularly desirable for certain flavors and fragrances and may be used in toiletries, cosmetics, household cleaners, room sprays, laundry, and fine fragrance applications, such as in perfumes and toilet water, dental hygiene products (such as toothpastes and mouthwashes), orally administered medications, and food products.

Considerable work is performed by many scientists relating to identifying new substances which can be used, alone or in combinations, to impart to, or enhance, the aroma or flavor of various consumable materials, including, e.g., cosmetics, cleaners, and foodstuffs. While there may be some trends in the relationship between chemical structure and flavor or fragrance—such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile. The art of flavor and fragrance prediction is still in its infancy.

This application describes the surprising and unexpected olfactive qualities of new norbornane and norbornene derivatives, and their use as fragrance and flavor ingredients, and potential applications thereof.

### BRIEF SUMMARY

In one aspect, the application relates to compounds of Formula I or compounds of Formula II:

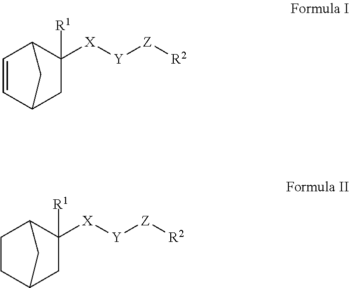

wherein:
R¹ is H, C₁₋₅alkyl, C₃₋₅cycloalkyl, OC₁₋₅alkyl, or OC₃₋₅cycloalkyl;
X is selected from O, C(R^{X1})(R^{X2}), C(O), C(O)O, C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Y is selected from a bond, C(R^{X1})(R^{X2}), C(O), C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Z is selected from a bond, O, CN, CHO, C(O)O, C(O), C(R^{X1})(R^{X2})O, C(R^{X1})(R^{X2})CN, C(R^{X1})(R^{X2})CHO, C(R^{X1})(R^{X2})C(O), C(R^{X1})(R^{X2})OC(O), and C(R^{X1})(R^{X2})C(O)O, provided that when Z is CN, CHO, C(R^{X1})(R^{X2})CN, or C(R^{X1})(R^{X2})CHO, then R² is absent;
or wherein X—Y—Z—R² is:

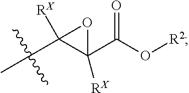

R² is H, OH, C₁₋₆alkyl, C₂₋₆alkenyl, C₃₋₆cycloalkyl, C₃₋₆cycloalkenyl, C₆₋₁₂bicycloalkyl, C₆₋₁₂bicycloalkenyl, aryl, or heteroaryl;
R^{X} is selected from H, C₁₋₆alkyl, C₂₋₆alkenyl, and C₃₋₆cycloalkyl;
R^{X1} and R^{X2} are each independently selected from H, OH, C₁₋₆alkyl, C₂₋₆alkenyl, and C₃₋₆cycloalkyl;
   provided that the group X—Y—Z— does not form a peroxide (—O—O—) or acid anhydride ((C(O)—O—C(O)) structure; and
optionally provided that Y is not C(O), C(R^{X1})(R^{X2})O, or C(R^{X1})(R^{X2})C(O), when Z is O, CN, CHO, C(O)O, or C(O); and
optionally provided that X—Y—Z—R² is not —O—R², —OC(O)—R², or —O—C(R^{X})(R^{X})C(R^{X})(R^{X})—CHO when R^{X1} and R² are each H, methyl, or ethyl; and
optionally provided that Y and Z are not both bonds when X is C(R^{X1})(R^{X2})C(R^{X1})(R^{X2}) or C(R^{X})═C(R^{X});
and all enantiomers, diastereomers, endo/exo, and E/Z isomers thereof;
in free or salt form;
optionally provided that the compound is not one or more of the Selected Compounds as defined hereinbelow,

In another aspect, the application relates to fragrance and flavor compositions comprising the compound of Formula I or II, optionally comprising one or more additives, additional fragrance or flavor ingredients, or a combination of additives and fragrance or flavor ingredients. In some embodiments, the composition comprises one or more of the Selected Compounds, as defined hereinbelow.

In another aspect, the application relates to products, such as consumer products, comprising such fragrance and flavor compositions comprising the compound of Formula I or II as herein provided. In some embodiments, the product comprises one or more of the Selected Compounds, as defined hereinbelow.

In another aspect, the present disclosure provides a method of making compounds of Formula I or II. In some embodiments, the method relates to making one or more of the Selected Compounds, as defined hereinbelow.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

The inventors have unexpectedly found that the compounds disclosed herein have unique and favorable aromas. The compounds are thus useful in imparting and providing desirable aromas and/or flavors to the products to which they are added, including, but not limited to, perfumes, household products, laundry products, personal care products, cosmetics, dental hygiene products, orally administered medications, and food products. The Compounds of Formula I or II may be employed in varying amounts depending upon the specific fragrance or flavor product application, the nature and amount of other flavor or fragrance ingredients present, and the desired aroma and/or flavor of the product.

In a first aspect, the present disclosure provides a compound of Formula I or Formula II:

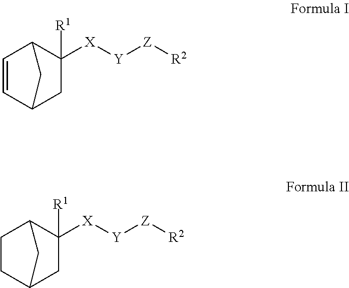

wherein:
R¹ is H, C₁₋₅alkyl, C₃₋₅cycloalkyl, OC₁₋₅alkyl, or OC₃₋₅cycloalkyl;
X is selected from O, C(R^{X1})(R^{X2}), C(O), C(O)O, C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Y is selected from a bond, C(R^{X1})(R^{X2}), C(O), C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Z is selected from a bond, O, CN, CHO, C(O)O, C(O), C(R^{X1})(R^{X2})O, C(R^{X1})(R^{X2})CN, C(R^{X1})(R^{X2})CHO, C(R^{X1})(R^{X2})C(O), C(R^{X1})(R^{X2})OC(O), and C(R^{X1})(R^{X2})C(O)O, provided that when Z is CN, CHO, C(R^{X1})(R^{X2})CN, or C(R^{X1})(R^{X2})CHO, then R² is absent;
or wherein X—Y—Z—R² is:

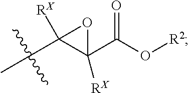

R² is H, OH, C₁₋₆alkyl, C₂₋₆alkenyl, C₃₋₆cycloalkyl, C₃₋₆cycloalkenyl, C₆₋₁₂bicycloalkyl, C₆₋₁₂bicycloalkenyl, aryl, or heteroaryl, or when Z is CN, CHO, C(R^{X1})(R^{X2})CN, or C(R^{X1})(R^{X2})CHO, then R² is absent;
R^{X} is selected from H, C₁₋₆alkyl, C₂₋₆alkenyl, and C₃₋₆cycloalkyl;
R^{X1} and R^{X2} are each independently selected from H, OH, C₁₋₆alkyl, C₂₋₆alkenyl, and C₃₋₆cycloalkyl;
   provided that the group X—Y—Z— does not form a peroxide (—O—O—) or acid anhydride ((C(O)—O—C(O)) structure; and
optionally provided that Y is not C(O), C(R^{X1})(R^{X2})O, or C(R^{X1})(R^{X2})C(O), when Z is O, CN, CHO, C(O)O, or C(O); and
optionally provided that X—Y—Z—R² is not —O—R², —OC(O)—R², or —O—C(R^{X})(R^{X})C(R^{X})(R^{X})—CHO when R^{X1} and R² are each H, methyl, or ethyl; and
optionally provided that Y and Z are not both bonds when X is C(R^{X1})(R^{X2})C(R^{X1})(R^{X2}) or C(R^{X})═C(R^{X});
and all enantiomers, diastereomers, endo/exo, and E/Z isomers thereof;
in free or salt form;
optionally provided that the compound is not one or more of the Selected Compounds as defined hereinbelow.

In some embodiments, excluded from the Compound of Formula I and/or the Compound of Formula II are compounds wherein Y is C(O), C(R^{X1})(R^{X2})O, or C(R^{X1})(R^{X2})C(O), when Z is O, CN, CHO, C(O)O, or C(O).

In some embodiments, excluded from the Compound of Formula I and/or the Compound of Formula II are compounds wherein X—Y—Z—R² is —O—R², —OC(O)—R², or —O—C(R^{X1})(R^{X2})C(R^{X1})(R^{X2})—CHO when R^{X1} and R² are each H, methyl, or ethyl.

In some embodiments, excluded from the Compound of Formula I and/or the Compound of Formula II are compounds wherein Y and Z are both bonds when X is C(R^{X1})(R^{X2})C(R^{X1})(R^{X2}) or C(R^{X})═C(R^{X}).

In further embodiments, of the first aspect, the present disclosure provides:
1.1 Compound of Formula I, wherein R¹ is H;
1.2 Compound of Formula I, wherein R¹ is C₁₋₅alkyl (e.g., methyl, ethyl);
1.3 Compound of Formula I, wherein R¹ is C₃₋₅cycloalkyl (e.g., cyclopropyl);
1.4 Compound of Formula I, wherein R¹ is OC₁₋₅alkyl (e.g., methoxy, ethoxy);
1.5 Compound of Formula I, wherein R¹ is OC₃₋₅cycloalkyl (e.g., cyclopropoxy);
1.6 Compound of Formula I or any of 1.1-1.5, wherein X is O;
1.7 Compound of Formula I or any of 1.1-1.5, wherein X is C(R^{X1})(R^{X2}) (e.g., CH₂);
1.8 Compound of Formula I or any of 1.1-1.5, wherein X is C(O);
1.9 Compound of Formula I or any of 1.1-1.5, wherein X is C(O)O;
1.10 Compound of Formula I or any of 1.1-1.5, wherein X is C(R^{X1})(R^{X2})C(R^{X1})(R^{X2}), e.g., CH₂CH₂, CH(Me)CH₂, or CH₂CH(Me);
1.11 Compound of Formula I or any of 1.1-1.5, wherein X is C(R^{X})═C(R^{X}) (e.g., in E- or Z-configuration), e.g., CH═CH, C(Me)=CH, or CH═C(Me);
1.12 Compound of Formula I or any of 1.1-1.5, wherein X is C(R^{X1})(R^{X2})O;
1.13 Compound of Formula I or any of 1.1-1.5, wherein X is OC(R^{X1})(R^{X2});
1.14 Compound of Formula I or any of 1.1-1.5, wherein X is C(R^{X1})(R^{X2})C(O);
1.15 Compound of Formula I or any of 1.1-1.5, wherein X is C(O)C(R^{X1})(R^{X2});
1.16 Compound of Formula I or any of 1.1-1.15, wherein Y is a bond;
1.17 Compound of Formula I or any of 1.1-1.15, wherein Y is C(R^{X1})(R^{X2});
1.18 Compound of Formula I or any of 1.1-1.15, wherein Y is C(O);
1.19 Compound of Formula I or any of 1.1-1.15, wherein Y is C(R^{X1})(R^{X2})C(R^{X1})(R^{X2}), e.g., CH₂CH₂, CH(Me)CH₂, or CH₂CH(Me);
1.20 Compound of Formula I or any of 1.1-1.15, wherein Y is C(R^{X})═C(R^{X}) (e.g., in E- or Z-configuration), e.g., CH═CH, C(Me)=CH, or CH═C(Me);
1.21 Compound of Formula I or any of 1.1-1.15, wherein Y is C(R^{X1})(R^{X2})O;
1.22 Compound of Formula I or any of 1.1-1.15, wherein Y is OC(R^{X1})(R^{X2});
1.23 Compound of Formula I or any of 1.1-1.15, wherein Y is C(R^{X1})(R^{X2})C(O);
1.24 Compound of Formula I or any of 1.1-1.15, wherein Y is C(O)C(R^{X1})(R^{X2});
1.25 Compound of Formula I or any of 1.1-1.24, wherein Z is a bond;
1.26 Compound of Formula I or any of 1.1-1.24, wherein Z is O;
1.27 Compound of Formula I or any of 1.1-1.24, wherein Z is CN;
1.28 Compound of Formula I or any of 1.1-1.24, wherein Z is CHO;
1.29 Compound of Formula I or any of 1.1-1.24, wherein Z is C(O)O;
1.30 Compound of Formula I or any of 1.1-1.24, wherein Z is C(O);
1.31 Compound of Formula I or any of 1.1-1.24, wherein Z is C(R^{X1})(R^{X2})O;
1.32 Compound of Formula I or any of 1.1-1.24, wherein Z is C(R^{X1})(R^{X2})CN;
1.33 Compound of Formula I or any of 1.1-1.24, wherein Z is C(R^{X1})(R^{X2})CHO;
1.34 Compound of Formula I or any of 1.1-1.24, wherein Z is C(R^{X1})(R^{X2})C(O);
1.35 Compound of Formula I or any of 1.1-1.24, wherein Z is C(R^{X1})(R^{X2})OC(O);
1.36 Compound of Formula I or any of 1.1-1.24, wherein Z is C(R^{X1})(R^{X2})C(O)O;
1.37 Compound of Formula I or any of 1.1-1.5, wherein X—Y—Z—R² is:

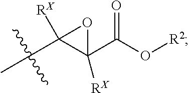

1.38 Compound of Formula I or any of 1.1-1.37, wherein one or more of R^{X}, R^{X1} and R^{X2} are H;
1.39 Compound of Formula I or any of 1.1-1.38, wherein one or more of R^{X}, R^{X1} and R^{X2} are OH;
1.40 Compound of Formula I or any of 1.1-1.39, wherein one or more of R^{X}, R^{X1} and R^{X2} are C₁₋₆alkyl (e.g., methyl or ethyl);
1.41 Compound of Formula I or any of 1.1-1.40, wherein one or more of R^{X}, R^{X1} and R^{X2} are C₃₋₆cycloalkyl (e.g., cyclopropyl);
1.42 Compound of Formula I or any of 1.1-1.41, wherein one or more of R^{X}, R^{X1} and R^{X2} are C₂₋₆alkenyl (e.g., vinyl or allyl), optionally in E- or Z-stereochemistry;
1.43 Compound of Formula I or any of 1.1-1.37, wherein R^{X}, R^{X1} and R² are each independently selected from H and C₁₋₆alkyl (e.g., methyl or ethyl);
1.44 Compound of Formula I or any of 1.1-1.37, wherein R^{X}, R^{X1} and R² are each independently selected from H and methyl;
1.45 Compound of Formula I or any of 1.1-1.37, wherein R^{X}, R^{X1} and R² are each H;
1.46 Compound of Formula I or any of 1.1-1.37, wherein R^{X}, R^{X1} and R² are each methyl;
1.47 Compound of Formula I or any of 1.1-1.46, wherein R² is H;
1.48 Compound of Formula I or any of 1.1-1.46, wherein R² is OH;
1.49 Compound of Formula I or any of 1.1-1.46, wherein R² is C₁₋₆alkyl;
1.50 Compound 1.49, wherein R² is selected from methyl, ethyl, propyl, isopropyl, n-butyl, sec-butyl, isobutyl, tert-butyl, n-pentyl, isopentyl, neopentyl, 3-pentyl, 3-methyl-2-butyl, n-hexyl, and 2-methyl-3-pentyl;
1.51 Compound of Formula I or any of 1.1-1.46, wherein R² is C₃₋₆cycloalkyl;
1.52 Compound 1.51, wherein R² is cyclopropyl, cyclobutyl, or cyclopentyl;
1.53 Compound of Formula I or any of 1.1-1.46, wherein R² is C₂₋₆alkenyl, optionally in E- or Z-stereochemistry;
1.54 Compound 1.53, wherein R² is vinyl, allyl, crotyl, methallyl, 3,3-dimethylallyl, 1-propen-1-yl, 2-buten-1-yl, 2-buten-2-yl, 3-buten-1-yl, 2-penten-2-yl, or 3-hexen-1-yl;
1.55 Compound of Formula I or any of 1.1-1.46, wherein R² is C₃₋₆cycloalkenyl (e.g., cyclopentenyl, or cyclohexenyl);
1.56 Compound of Formula I or any of 1.1-1.46, wherein R² is C₆₋₁₂bicycloalkyl (e.g., bicyclo[4.2.0]octanyl, bicyclo[4.3.0]nonanyl, bicyclo[4.4.0]decanyl, bicyclo[2.2.1]heptanyl, bicyclo[2.2.2]octanyl, bicyclo[3.2.2]nonanyl);
1.57 Compound of Formula I or any of 1.1-1.46, wherein R² is C₆₋₁₂bicycloalkenyl (e.g., bicyclo[2.2.1]hept-2-en-2-yl, bicyclo[2.2.1]hept-5-en-2-yl, bicyclo[2.2.2]octen-5-en-2-yl, bicyclo[2.2.2]oct-2-en-2-yl);
1.58 Compound of Formula I or any of 1.1-1.46, wherein R² is aryl (e.g., phenyl, naphthyl);
1.59 Compound of Formula I or any of 1.1-1.46, wherein R² is heteroaryl;
1.60 Compound 1.59, wherein R² is 5-membered heteroaryl (e.g., thiophenyl, furanyl, pyrrolyl, oxazolyl, isoxazolyl, pyrazolyl, imidazolyl, thiazolyl, or isothiazolyl);
1.61 Compound 1.59, wherein R² is 6-membered heteroaryl (e.g., pyridinyl, pyrimidinyl, pyrazinyl, or pyridazinyl);
1.62 Compound 1.59, wherein R² is 9- to 12-membered bicyclic heteroaryl (e.g., benzofuranyl, benzothiophenyl, indolyl, indazolyl, benzimidazolyl, benzoxazolyl, benzothiazolyl)
1.63 Compound of Formula I, or any of 1.1-1.62, wherein the compound is a Compound of Formula Ia:

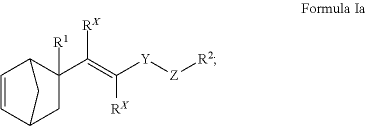

wherein Y is C(R^{X1})(R^{X2})C(R^{X1})(R^{X2}) or a bond, Z is CHO, C(O)O, C(O), C(R^{X1})(R^{X2})O, or C(R^{X1})(R^{X2})OC(O), and R² is H, C₁₋₅alkyl, C₃₋₅cycloalkyl, or C₂₋₅alkenyl;
1.64 Compound 1.63, wherein R^{X} is independently selected from H and C₁₋₆alkyl (e.g., methyl);
1.65 Compound 1.63, wherein R^{X} is independently selected from H and methyl;
1.66 Compound 1.63, wherein R^{X} is H;
1.67 Any one of Compounds 1.63-1.66, wherein R^{X1} and R^{X2} are each independently selected from H and C₁₋₆alkyl (e.g., methyl or ethyl);
1.68 Any one of Compounds 1.63-1.66, wherein R^{X1} and R^{X2} are each independently selected from H, methyl, and ethyl, e.g., R^{X1} and R² are each H;
1.69 Any one of Compounds 1.63-1.66, wherein Y is selected from —CH₂CH(Me)-, —CH(Me)CH₂—, —CH(Me)CH(Me)-, —CH₂C(Me)₂-, —C(Me)₂CH₂—, —CH₂CH(Et)-, —CH(Et)CH₂—;
1.70 Any one of Compounds 1.63-1.66, wherein Y is —CH₂CH(Me)-;
1.71 Any one of Compounds 1.63-1.66, wherein Y is a bond;
1.72 Any one of Compounds 1.63-1.71, wherein Z is CHO;
1.73 Any one of Compounds 1.63-1.71, wherein Z is C(R^{X1})(R^{X2})O, optionally wherein R² is H;
1.74 Compound 1.73, wherein R^{X1} and R^{X2} are each independently selected from H and C₁₋₆alkyl (e.g., methyl);
1.75 Any one of Compounds 1.63-1.71, wherein Z is C(O)O, C(O), or C(R^{X1})(R^{X2})OC(O);
1.76 Compound 1.75, wherein R^{X1} and R^{X2} are each independently selected from H and C₁₋₆alkyl (e.g., methyl);
1.77 Any one of Compounds 1.63-1.76, wherein R² is H;
1.78 Any one of Compounds 1.63-1.76, wherein R² is C₁₋₆alkyl (e.g., methyl, ethyl, propyl, isopropyl);
1.79 Any one of Compounds 1.63-1.76, wherein R² is C₃₋₆cycloalkyl (e.g., cyclopropyl, cyclobutyl);
1.80 Any one of Compounds 1.63-1.76, wherein R² is C₂₋₆alkenyl (e.g., vinyl, allyl, methallyl), optionally in E- or Z-stereochemistry;
1.81 Any one of Compounds 1.63-1.80, wherein Z is C(O)O and R² is C₁₋₆alkyl (e.g., methyl, ethyl, propyl, isopropyl);
1.82 Any one of Compounds 1.63-1.80, wherein Z is C(O)O and R² is C₃₋₆cycloalkyl (e.g., cyclopropyl, cyclobutyl);
1.83 Any one of Compounds 1.63-1.80, wherein Z is C(O)O and R² is C₂₋₆alkenyl (e.g., vinyl, allyl, methallyl), optionally in E- or Z-stereochemistry;
1.84 Any one of Compounds 1.63-1.80, wherein Z is C(O)O and R² is methyl, ethyl, cyclopropyl, propyl, or isopropyl;
1.85 Any one of Compounds 1.63-1.84, wherein Z is CH₂O and R² is H;
1.86 Any one of Compounds 1.63-1.85, wherein R¹ is H or methyl;
1.87 Compound of Formula I, or any of 1.1-1.62, wherein the compound is a Compound of Formula Ib:

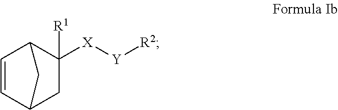

wherein X is C(R^{X1})(R^{X2})O, or C(O)(O), and Y is C(R^{X1})(R^{X2}) or C(O), provided that X is not C(O)O when Y is C(O);
1.88 Compound 1.87, wherein X is C(R^{X1})(R^{X2})O;
1.89 Compound 1.88, wherein R^{X1} and R^{X2} are each independently selected from H, C₁₋₆alkyl (e.g., methyl or ethyl), and C₂₋₅alkenyl (e.g., vinyl, allyl), optionally in E- or Z-stereochemistry;
1.90 Compound 1.89, wherein R^{X1} and R^{X2} are each independently selected from H, methyl, ethyl, and vinyl;
1.91 Compound 1.89, wherein C(R^{X1})(R^{X2})O is CH₂O, CH(Me)O, or CH(CH═CH₂)O;
1.92 Compound 1.87, wherein X is C(O)O;
1.93 Any of Compounds 1.87-1.92, wherein Y is C(R^{X1})(R^{X2});
1.94 Compound 1.93, wherein R^{X1} and R^{X2} are each independently selected from H C₁₋₆alkyl (e.g., methyl or ethyl), and C₂₋₅alkenyl (e.g., vinyl, allyl), optionally in E- or Z-stereochemistry;
1.95 Compound 1.94, wherein C(R^{X1})(R^{X2})O is CH₂O;
1.96 Any of Compounds 1.87-1.92, wherein Y is C(O);
1.97 Any of Compounds 1.87-1.96, wherein R² is H;
1.98 Any one of Compounds 1.87-1.96, wherein R² is C₁₋₆alkyl (e.g., methyl, ethyl, propyl, isopropyl);
1.99 Compound 1.98, wherein R² is selected from methyl, ethyl, propyl, isopropyl, n-butyl, sec-butyl, isobutyl, n-pentyl, 3-pentyl, 3-methyl-2-butyl, n-hexyl, and 2-methyl-3-pentyl;
1.100 Any one of Compounds 1.87-1.96, wherein R² is C₃₋₆cycloalkyl (e.g., cyclopropyl, cyclobutyl);
1.101 Any one of Compounds 1.87-1.96, wherein R² is C₂₋₆alkenyl (e.g., vinyl, allyl, methallyl), optionally in E- or Z-stereochemistry;
1.102 Compound 1.101, wherein R² is vinyl, allyl, crotyl, methallyl, 3,3-dimethylallyl, 1-propen-1-yl, 2-buten-1-yl, 2-buten-2-yl, 3-buten-1-yl, 2-penten-2-yl, or 3-hexen-1-yl;
1.103 Any one of Compounds 1.87-1.96, wherein R² is C₆₋₁₂bicycloalkyl or C₆₋₁₂bicycloalkenyl (e.g., bicyclo[2.2.1]heptanyl, bicyclo[2.2.1]hept-2-en-2-yl, bicyclo[2.2.1]hept-5-en-2-yl);
1.104 Any one of Compounds 1.87-1.96, wherein R² is heteroaryl;
1.105 Compound 1.104, wherein R² is thiophenyl, furanyl, or pyridyl;
1.106 Any one of Compounds 1.87-1.96, wherein R² is aryl (e.g., phenyl);
1.107 Any one of Compounds 1.87-1.1064, wherein R¹ is H or methyl;
1.108 Compound of Formula I, or any of 1.1-1.62, wherein the compound is a Compound of Formula Ic:

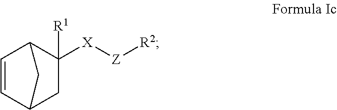

wherein X is C(R^{X1})(R^{X2}) or C(R^{X1})(R^{X2})C(R^{X1})(R^{X2}), and Z is CN, CHO, C(R^{X1})(R^{X2})CN, C(R^{X1})(R^{X2})CHO, C(R^{X1})(R^{X2})O, and C(R^{X1})(R^{X2})C(O)O;
1.109 Compound 1.108, wherein X is C(R^{X1})(R^{X2});
1.110 Compound 1.109, wherein R^{X1} and R^{X2} are each independently selected from H, C₁₋₆alkyl (e.g., methyl or ethyl), and C₂₋₅alkenyl (e.g., vinyl, allyl), optionally in E- or Z-stereochemistry;
1.111 Compound 1.109, wherein R^{X1} and R^{X2} are each independently selected from H, and C₁₋₆alkyl (e.g., methyl or ethyl);
1.112 Compound 1.109, wherein R^{X1} and R^{X2} are each independently selected from H, methyl, and ethyl, e.g., wherein X is CH₂, CH(Me), or CH(Et);
1.113 Compound 1.108, wherein X is C(R^{X1})(R^{X2})C(R^{X1})(R^{X2});
1.114 Compound 1.113, wherein each R^{X1} and R² are each independently selected from H, C₁₋₆alkyl (e.g., methyl or ethyl), and C₂₋₅alkenyl (e.g., vinyl, allyl), optionally in E- or Z-stereochemistry;
1.115 Compound 1.114, wherein each R^{X1} and R² are each independently selected from H, and C₁₋₆alkyl (e.g., methyl or ethyl);
1.116 Compound 1.114, wherein each R^{X1} and R² are each independently selected from H, methyl, and ethyl;
1.117 Compound 1.116, wherein X is CH₂CH₂, CH₂CH(Me), CH₂CH(Et), CH(Me)CH₂, or CH(Et)CH₂;
1.118 Any of Compounds 1.108-1.117, wherein Z is CN and R² is absent;
1.119 Any of Compounds 1.108-1.117, wherein Z is CHO and R² is absent;
1.120 Any of Compounds 1.108-1.117, wherein Z is C(R^{X1})(R^{X2})CN and R² is absent;
1.121 Any of Compounds 1.108-1.117, wherein Z is C(R^{X1})(R^{X2})CHO and R² is absent;
1.122 Any of Compounds 1.108-1.117, wherein Z is C(R^{X1})(R^{X2})C(O)O, and R² is C₁₋₆alkyl (e.g., methyl, ethyl, propyl, isopropyl);
1.123 Any of Compounds 1.108-1.117, wherein Z is C(R^{X1})(R^{X2})C(O)O, and R² is C₂₋₆alkenyl (e.g., vinyl, allyl, methallyl), optionally in E- or Z-stereochemistry;
1.124 Any of Compounds 1.108-1.117, wherein Z is C(R^{X1})(R^{X2})O, and R² is H;
1.125 Compound 1.120, 1.121, 1.122, 1.123, or 1.124, wherein R^{X1} and R^{X2} are each independently selected from H and methyl, e.g., wherein the C(R^{X1})(R^{X2}) of Z is CH₂ or CH(Me);
1.126 Any one of Compounds 1.108-1.125, wherein R¹ is H or methyl;
1.127 Compound of Formula I, wherein the compound is a Compound of Formula Id:

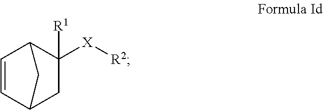

wherein X is C(R^{X1})(R^{X2}), C(R^{X1})(R^{X2})O, C(O) or C(O)(O), or wherein X—R² is:

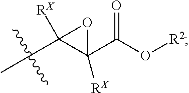

1.128 Compound of 1.127, wherein X is C(R^{X1})(R^{X2});
1.129 Compound of 1.127, wherein X is C(R^{X1})(R^{X2})O;
1.130 Compound 1.128 or 1.129, wherein R^{X1} and R^{X2} are each independently selected from H, C₁₋₆alkyl (e.g., methyl or ethyl), and C₂₋₅alkenyl (e.g., vinyl, allyl), optionally in E- or Z-stereochemistry;
1.131 Compound 1.130, wherein R^{X1} and R^{X2} are each independently selected from H, methyl, ethyl, and vinyl;
1.132 Compound 1.130, wherein X is CH₂O, CH(Me)O, or CH(CH═CH₂)O;
1.133 Compound 1.130, wherein X is CH₂, CH(Me), or CH(CH═CH₂);
1.134 Compound 1.127, wherein X is C(O);
1.135 Compound 1.127, wherein X is C(O)O;
1.136 Compound 1.127, wherein X—R² is:

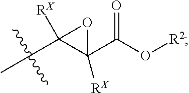

1.137 Any of Compounds 1.127-1.136, wherein R² is C₁₋₆alkyl (e.g., methyl, ethyl, propyl, isopropyl);
1.138 Compound 1.137, wherein R² is selected from methyl, ethyl, propyl, isopropyl, n-butyl, sec-butyl, isobutyl, n-pentyl, 3-pentyl, 3-methyl-2-butyl, n-hexyl, and 2-methyl-3-pentyl;
1.139 Any one of Compounds 1.127-1.136, wherein R² is C₃₋₆cycloalkyl (e.g., cyclopropyl, cyclobutyl);
1.140 Any one of Compounds 1.127-1.136, wherein R² is C₂₋₆alkenyl (e.g., vinyl, allyl, methallyl), optionally in E- or Z-stereochemistry;
1.141 Compound 1.140, wherein R² is vinyl, allyl, crotyl, methallyl, 3,3-dimethylallyl, 1-propen-1-yl, 2-buten-1-yl, 2-buten-2-yl, 3-buten-1-yl, 2-penten-2-yl, or 3-hexen-1-yl;
1.142 Any one of Compounds 1.127-1.136, wherein R² is C₆₋₁₂bicycloalkyl or C₆₋₁₂bicycloalkenyl (e.g., bicyclo[2.2.1]heptanyl, bicyclo[2.2.1]hept-2-en-2-yl, bicyclo[2.2.1]hept-5-en-2-yl);
1.143 Any one of Compounds 1.127-1.136, wherein R² is heteroaryl;
1.144 Compound 1.143, wherein R² is 5-membered heteroaryl (e.g., thiophenyl, furanyl, pyrrolyl, oxazolyl, isoxazolyl, pyrazolyl, imidazolyl, thiazolyl, or isothiazolyl);
1.145 Any one of Compounds 1.127-1.136, wherein R² is aryl (e.g., phenyl);
1.146 Any one of Compounds 1.127-1.145, wherein R¹ is H or methyl;
1.147 Compound of Formula I, or any of 1.1-1.146, wherein the compound of Formula I is selected from any of the following:

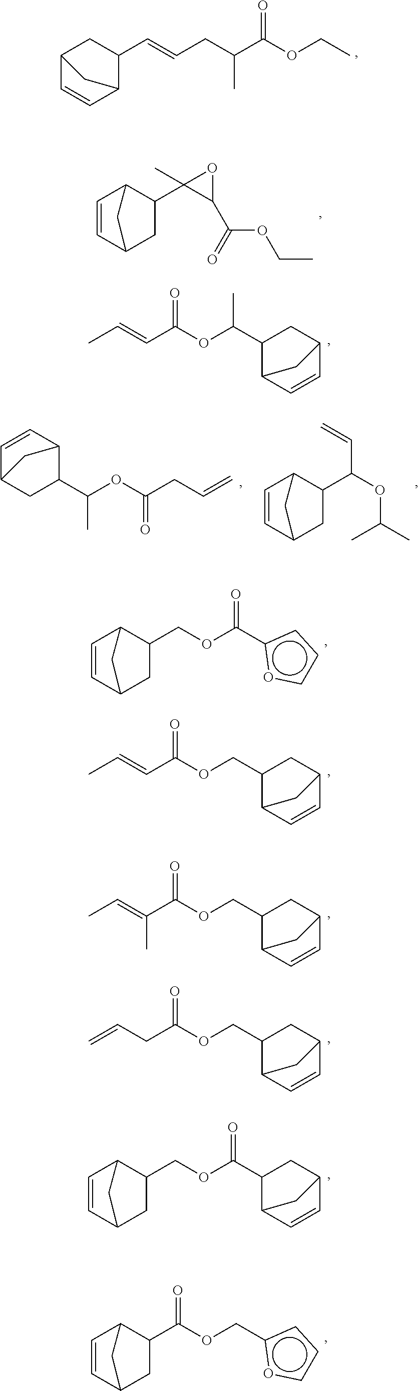

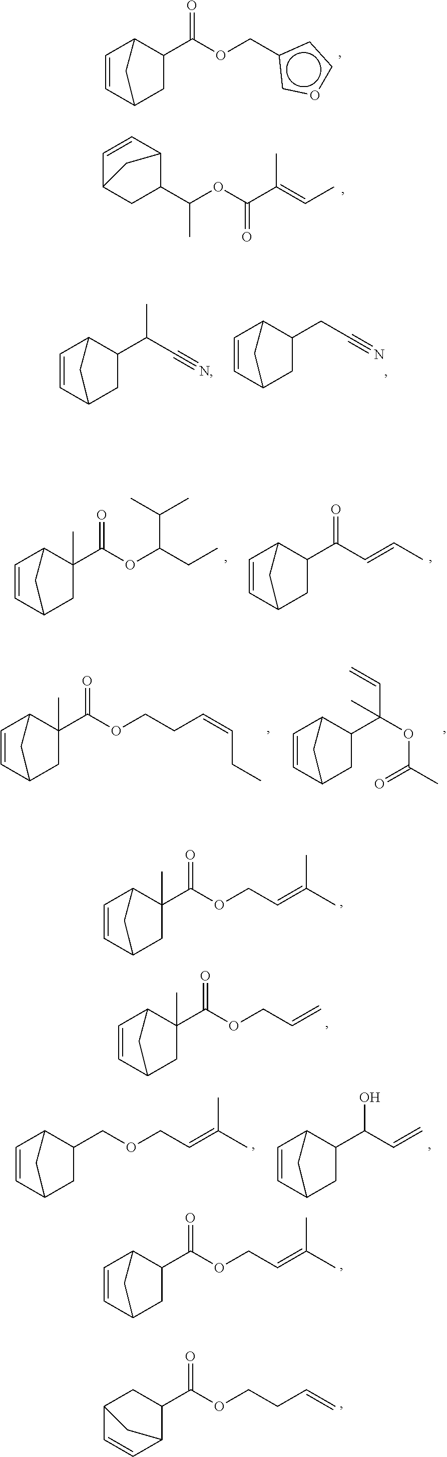

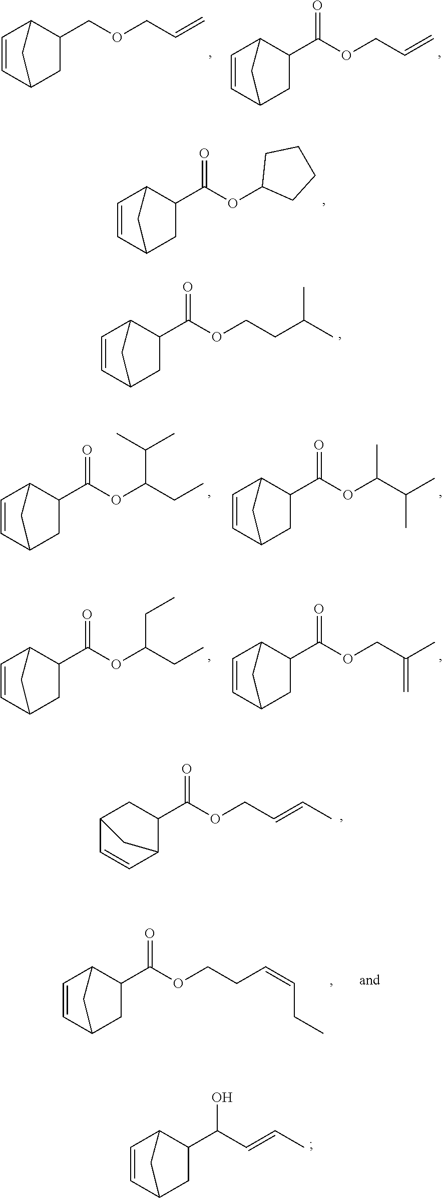

1.148 Compound of Formula I, or any of 1.1-1.146, wherein the compound of Formula I is selected from any of the following:

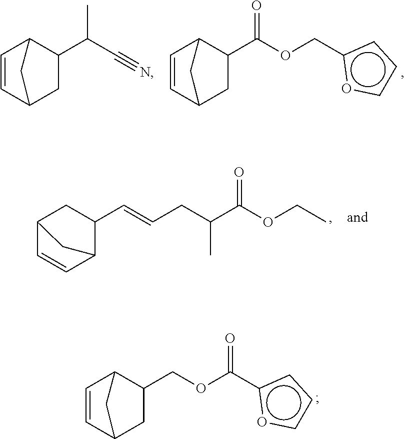

1.149 Compound of Formula I, or any of 1.1-1.146, wherein the compound of Formula I is:

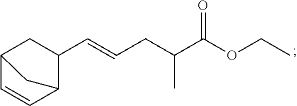 [SMILES: CCOC(=O)C(C)C/C=C/C1CC2C=CC1C2]

1.150 Compound of Formula I, or any of 1.1-1.146, wherein the compound of Formula I is selected from any of the following:

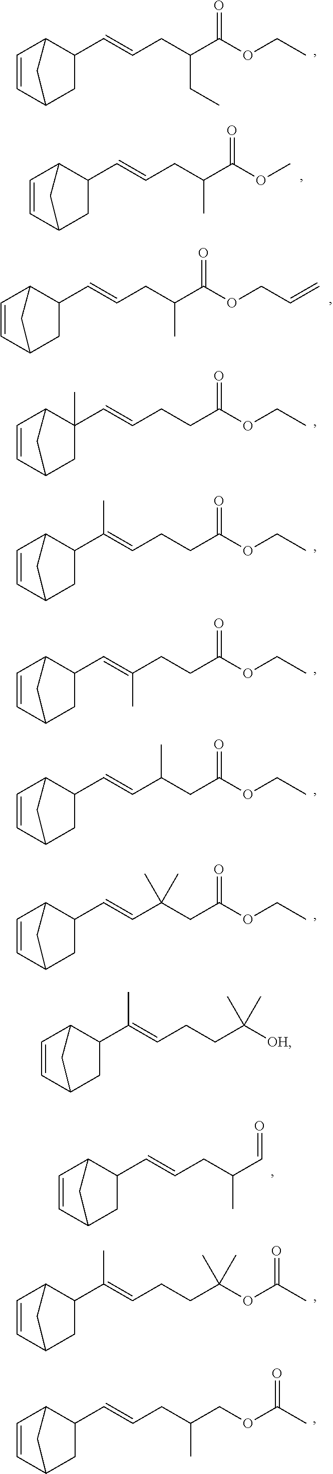

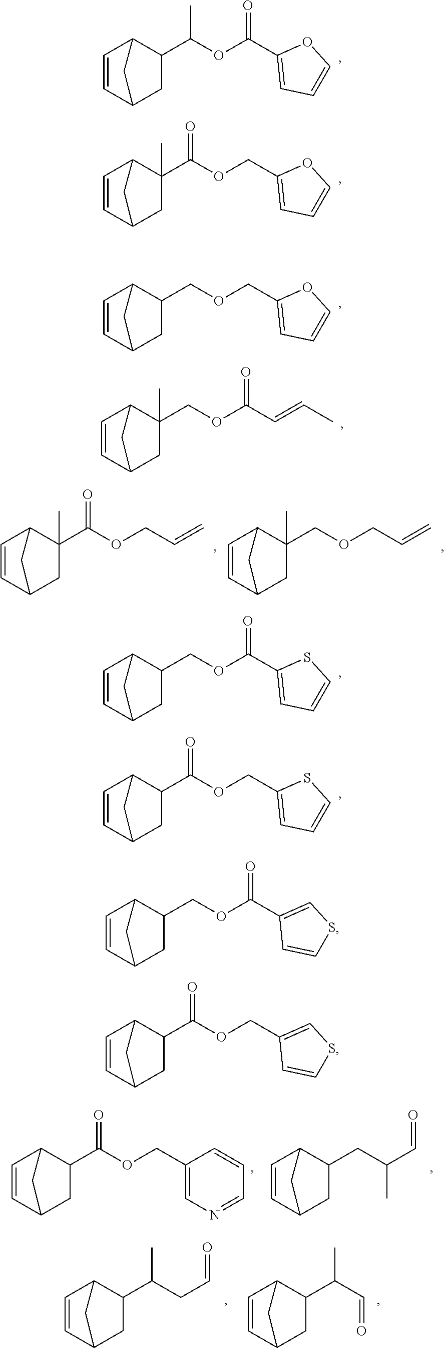

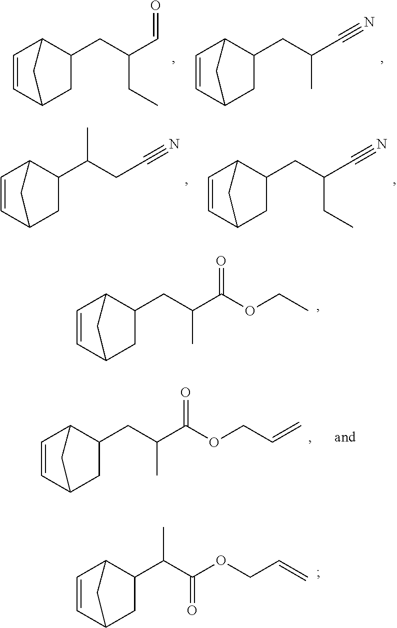

1.151 Compound of Formula I, or any of 1.1-1.62, wherein the compound is a Compound of Formula Ie:

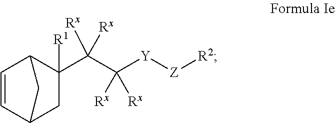

wherein R^{X} is independently selected from H, C₁₋₆alkyl, C₂₋₆alkenyl, and C₃₋₆cycloalkyl, Y is C(R^{X1})(R^{X2})C(R^{X1})(R^{X2}) or a bond, Z is CHO, C(O)O, C(O), C(R^{X1})(R^{X2})O, or C(R^{X1})(R^{X2})OC(O), and R² is H, C₁₋₅alkyl, C₃₋₅cycloalkyl, or C₂₋₅alkenyl;
1.152 Compound 1.151, wherein R^{X} is independently selected from H and C₁₋₆alkyl (e.g., methyl);
1.153 Compound 1.151, wherein R^{X} is independently selected from H and methyl;
1.154 Compound 1.151, wherein R^{X} is H;
1.155 Any one of Compounds 1.151-1.154, wherein R^{X1} and R^{X2} are each independently selected from H and C₁₋₆alkyl (e.g., methyl or ethyl);
1.156 Any one of Compounds 1.151-1.154, wherein R^{X1} and R^{X2} are each independently selected from H, methyl, and ethyl, e.g., R^{X1} and R² are each H;
1.157 Any one of Compounds 1.151-1.154, wherein Y is selected from —CH₂CH(Me)-, —CH(Me)CH₂—, —CH(Me)CH(Me)-, —CH₂C(Me)₂-, —C(Me)₂CH₂-, —CH₂CH(Et)-, —CH(Et)CH₂—;
1.158 Any one of Compounds 1.151-1.154, wherein Y is —CH₂CH(Me)-;
1.159 Any one of Compounds 1.151-1.154, wherein Y is a bond;
1.160 Any one of Compounds 1.151-1.159, wherein Z is CHO;
1.161 Any one of Compounds 1.151-1.159, wherein Z is C(R^{X1})(R^{X2})O, optionally wherein R² is H;
1.162 Compound 1.161, wherein R^{X1} and R^{X2} are each independently selected from H and C₁₋₆alkyl (e.g., methyl);
1.163 Any one of Compounds 1.151-1.159, wherein Z is C(O)O, C(O), or C(R^{X1})(R^{X2})OC(O);
1.164 Compound 1.163, wherein R^{X1} and R^{X2} are each independently selected from H and C₁₋₆alkyl (e.g., methyl);
1.165 Any one of Compounds 1.151-1.164, wherein R² is H;
1.166 Any one of Compounds 1.151-1.164, wherein R² is C₁₋₆alkyl (e.g., methyl, ethyl, propyl, isopropyl);
1.167 Any one of Compounds 1.151-1.164, wherein R² is C₃₋₆cycloalkyl (e.g., cyclopropyl, cyclobutyl);
1.168 Any one of Compounds 1.151-1.164, wherein R² is C₂₋₆alkenyl (e.g., vinyl, allyl, methallyl), optionally in E- or Z-stereochemistry;
1.169 Any one of Compounds 1.151-1.168, wherein Z is C(O)O and R² is C₁₋₆alkyl (e.g., methyl, ethyl, propyl, isopropyl);
1.170 Any one of Compounds 1.151-1.168, wherein Z is C(O)O and R² is C₃₋₆cycloalkyl (e.g., cyclopropyl, cyclobutyl);
1.171 Any one of Compounds 1.151-1.168, wherein Z is C(O)O and R² is C₂₋₆alkenyl (e.g., vinyl, allyl, methallyl), optionally in E- or Z-stereochemistry;
1.172 Any one of Compounds 1.151-1.168, wherein Z is C(O)O and R² is methyl, ethyl, cyclopropyl, propyl, or isopropyl;
1.173 Any one of Compounds 1.151-1.172, wherein Z is CH₂O and R² is H;
1.174 Any one of Compounds 1.151-1.173, wherein R¹ is H or methyl;
1.175 Compound of Formula I, or any of Compounds 1.1-1.146 or 1.151-1.174, wherein the compound of Formula I is selected from any of the following:

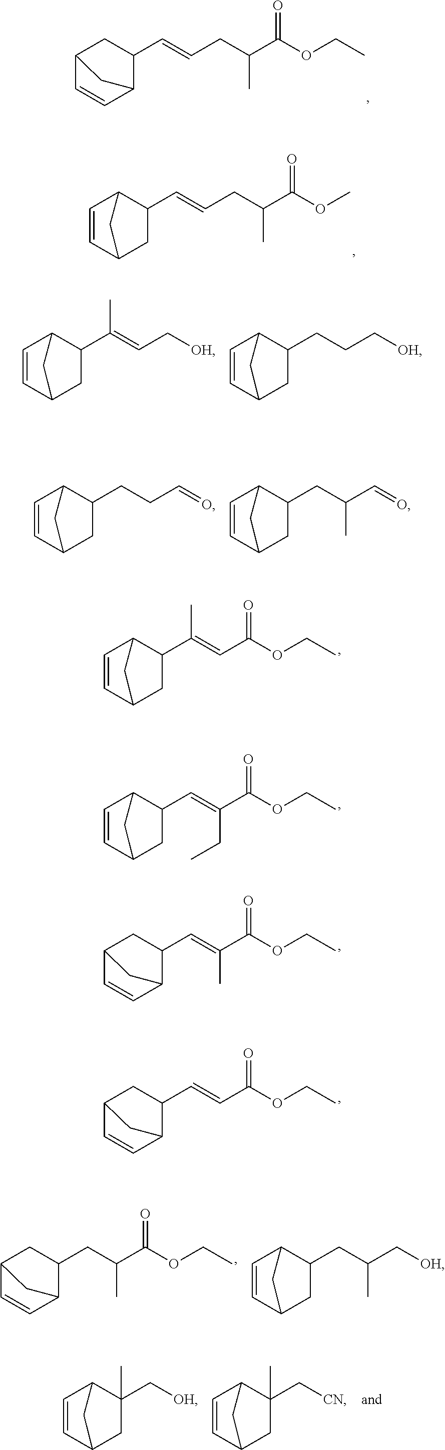

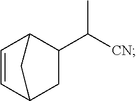

1.176 Compound of Formula I, or any of 1.1-1.62, wherein the compound is a Compound of Formula If:

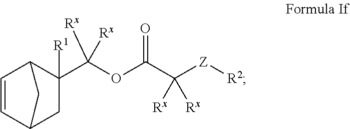

wherein R^{X} is independently selected from H, C₁₋₆alkyl, C₂₋₆alkenyl, and C₃₋₆cycloalkyl, Z is CN, CHO, C(O)O, or C(O), and R² is H, C₁₋₅alkyl, C₃₋₅cycloalkyl, or C₂₋₅alkenyl;
provided that when Z is CN or CHO, then R² is absent;
1.177 Compound 1.176, wherein R^{X} is independently selected from H and C₁₋₆alkyl (e.g., methyl or ethyl);
1.178 Compound 1.176, wherein R^{X} is independently selected from H, methyl, and ethyl;
1.179 Compound 1.176, wherein at least one R^{X} is independently C₁₋₆alkyl (e.g., methyl or ethyl);
1.180 Compound 1.176, wherein at least two R^{X} are independently C₁₋₆alkyl (e.g., methyl or ethyl), optionally wherein there are two C₁₋₆alkyl on different carbon atoms;
1.181 Any one of compounds 1.176-1.180, wherein one of the two C(R^{X}) moieties is a CH₂;
1.182 Any one of compounds 1.176-1.181, wherein at least one R^{X} is independently C₂₋₆alkenyl (e.g., vinyl);
1.183 Any one of compounds 1.176-1.182, wherein at least one R^{X} is independently C₃₋₆cycloalkyl (e.g., cyclopropyl);
1.184 Any one of compounds 1.176-1.181, wherein R^{X} is independently selected from H, methyl, ethyl, vinyl, and cyclopropyl;
1.185 Any one of compounds 1.176-1.184, wherein the Compound of Formula If is a Compound of Formula If-1:

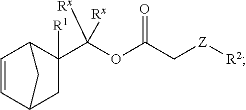

1.186 Any one of compounds 1.176-1.184, wherein the Compound of Formula If is a Compound of Formula If-2:

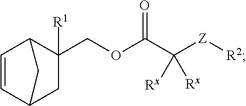

1.187 Any one of compounds 1.176-1.184, wherein the Compound of Formula If is a Compound of Formula If-3:

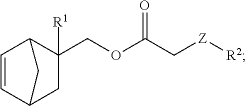

1.188 Any one of Compounds 1.176-1.187, wherein Z is CHO;
1.189 Any one of Compounds 1.176-1.187, wherein Z is CN;
1.190 Any one of Compounds 1.176-1.187, wherein Z is C(O)O;
1.191 Any one of Compounds 1.176-1.187, wherein Z is C(O);
1.192 Any one of Compounds 1.176-1.191, wherein R² is H;
1.193 Any one of Compounds 1.176-1.191, wherein R² is C₁₋₆alkyl (e.g., methyl, ethyl, propyl, isopropyl);
1.194 Any one of Compounds 1.176-1.191, wherein R² is C₃₋₆cycloalkyl (e.g., cyclopropyl, cyclobutyl);
1.195 Any one of Compounds 1.176-1.191, wherein R² is C₂₋₆alkenyl (e.g., vinyl, allyl, methallyl), optionally in E- or Z-stereochemistry;
1.196 Any one of Compounds 1.176-1.187, wherein Z is C(O)O and R² is C₁₋₆alkyl (e.g., methyl, ethyl, propyl, isopropyl);
1.197 Any one of Compounds 1.176-1.187, wherein Z is C(O)O and R² is C₃₋₆cycloalkyl (e.g., cyclopropyl, cyclobutyl);
1.198 Any one of Compounds 1.176-1.187, wherein Z is C(O)O and R² is C₂₋₆alkenyl (e.g., vinyl, allyl, methallyl), optionally in E- or Z-stereochemistry;
1.199 Any one of Compounds 1.176-1.187, wherein Z is C(O)O and R² is methyl, ethyl, cyclopropyl, propyl, or isopropyl;
1.200 Any one of Compounds 1.176-1.187, wherein Z is C(O) and R² is C₁₋₆alkyl (e.g., methyl, ethyl, propyl, isopropyl);
1.201 Any one of Compounds 1.176-1.187, wherein Z is C(O) and R² is C₃₋₆cycloalkyl (e.g., cyclopropyl, cyclobutyl);
1.202 Any one of Compounds 1.176-1.187, wherein Z is C(O) and R² is C₂₋₆alkenyl (e.g., vinyl, allyl, methallyl), optionally in E- or Z-stereochemistry;
1.203 Any one of Compounds 1.176-1.187, wherein Z is C(O) and R² is methyl, ethyl, cyclopropyl, propyl, or isopropyl;
1.204 Any one of Compounds 1.176-1.203, wherein R¹ is H;
1.205 Any one of Compounds 1.176-1.203, wherein R¹ is methyl;
1.206 Compound of Formula I, or any of Compounds 1.1-1.146 or 1.176-1.205, wherein the compound of Formula I is selected from any of the following:

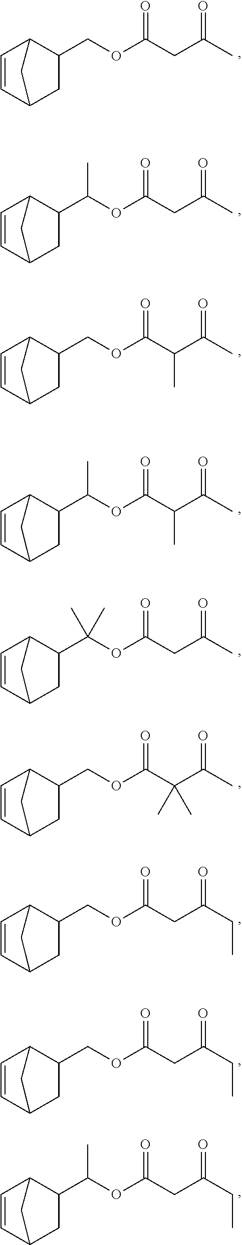

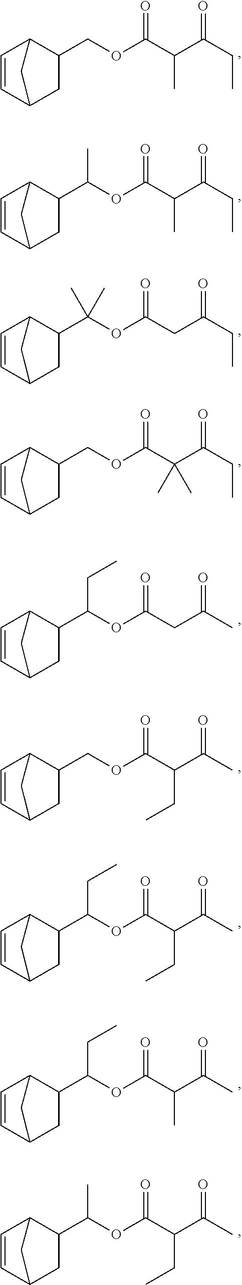

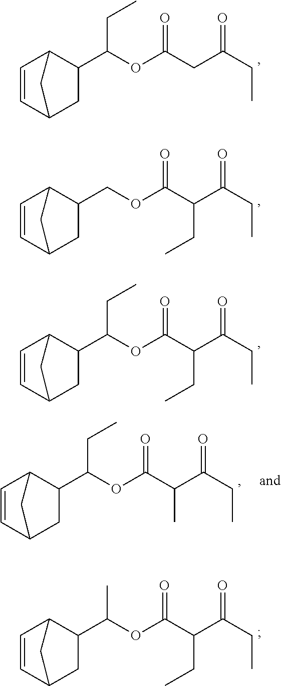

1.207 Any preceding compound, wherein the compound has endo stereochemistry about the norbornene ring;
1.208 Any preceding compound, wherein the compound has exo stereochemistry about the norbornene ring;
1.209 Any preceding compound, wherein the compound has a molecular weight selected from the range of to 110 to 600, or 110 to 500, or 110 to 450, or 110 to 400, or 110 to 350, or 110 to 300, or 110 to 250, or 110 to 200;
1.210 Any preceding compound, wherein the compound has a pleasing taste and/or aroma, e.g., as judged by a trained flavor or fragrance chemist or master perfumer (e.g., toasted coconut or other nutty aromas and/or tastes).

In further embodiments, of the first aspect, the present disclosure provides:
2.1. Compound of Formula II, wherein R¹ is H;
2.2. Compound of Formula II, wherein R¹ is C₁₋₅alkyl (e.g., methyl, ethyl);
2.3. Compound of Formula II, wherein R¹ is C₃₋₅cycloalkyl (e.g., cyclopropyl);
2.4. Compound of Formula II, wherein R¹ is OC₁₋₅alkyl (e.g., methoxy, ethoxy);
2.5. Compound of Formula II, wherein R¹ is OC₃₋₅cycloalkyl (e.g., cyclopropoxy);
2.6. Compound of Formula II or any of 2.1-2.5, wherein X is O;
2.7. Compound of Formula II or any of 2.1-2.5, wherein X is C(R^{X1})(R^{X2}), (e.g., CH₂);
2.8. Compound of Formula II or any of 2.1-2.5, wherein X is C(O);
2.9. Compound of Formula II or any of 2.1-2.5, wherein X is C(O)O;
2.10. Compound of Formula II or any of 2.1-2.5, wherein X is C(R^{X1})(R^{X2})C(R^{X1})(R^{X2}), e.g., CH₂CH₂, CH(Me)CH₂, or CH₂CH(Me);
2.11. Compound of Formula II or any of 2.1-2.5, wherein X is C(R^{X})═C(R^{X}) (e.g., in E- or Z-configuration), e.g., CH═CH, C(Me)=CH, or CH═C(Me);
2.12. Compound of Formula II or any of 2.1-2.5, wherein X is C(R^{X1})(R^{X2})O;
2.13. Compound of Formula II or any of 2.1-2.5, wherein X is OC(R^{X1})(R^{X2});
2.14. Compound of Formula II or any of 2.1-2.5, wherein X is C(R^{X1})(R^{X2})C(O);
2.15. Compound of Formula II or any of 2.1-2.5, wherein X is C(O)C(R^{X1})(R^{X2});
2.16. Compound of Formula II or any of 2.1-2.15, wherein Y is a bond;
2.17. Compound of Formula II or any of 2.1-2.15, wherein Y is C(R^{X1})(R^{X2});
2.18. Compound of Formula II or any of 2.1-2.15, wherein Y is C(O);
2.19. Compound of Formula II or any of 2.1-2.15, wherein Y is C(R^{X1})(R^{X2})C(R^{X1})(R^{X2}), e.g., CH₂CH₂, CH(Me)CH₂, or CH₂CH(Me);
2.20. Compound of Formula II or any of 2.1-2.15, wherein Y is C(R^{X})═C(R^{X}) (e.g., in E- or Z-configuration), e.g., CH═CH, C(Me)=CH, or CH═C(Me);
2.21. Compound of Formula II or any of 2.1-2.15, wherein Y is C(R^{X1})(R^{X2})O;
2.22. Compound of Formula II or any of 2.1-2.15, wherein Y is OC(R^{X1})(R^{X2});
2.23. Compound of Formula II or any of 2.1-2.15, wherein Y is C(R^{X1})(R^{X2})C(O);
2.24. Compound of Formula II or any of 2.1-2.15, wherein Y is C(O)C(R^{X1})(R^{X2});
2.25. Compound of Formula II or any of 2.1-2.24, wherein Z is a bond;
2.26. Compound of Formula II or any of 2.1-2.24, wherein Z is O;
2.27. Compound of Formula II or any of 2.1-2.24, wherein Z is CN;
2.28. Compound of Formula II or any of 2.1-2.24, wherein Z is CHO;
2.29. Compound of Formula II or any of 2.1-2.24, wherein Z is C(O)O;
2.30. Compound of Formula II or any of 2.1-2.24, wherein Z is C(O);
2.31. Compound of Formula II or any of 2.1-2.24, wherein Z is C(R^{X1})(R^{X2})O;
2.32. Compound of Formula II or any of 2.1-2.24, wherein Z is C(R^{X1})(R^{X2})CN;
2.33. Compound of Formula II or any of 2.1-2.24, wherein Z is C(R^{X1})(R^{X2})CHO;
2.34. Compound of Formula II or any of 2.1-2.24, wherein Z is C(R^{X1})(R^{X2})C(O);
2.35. Compound of Formula II or any of 2.1-2.24, wherein Z is C(R^{X1})(R^{X2})OC(O);
2.36. Compound of Formula II or any of 2.1-2.24, wherein Z is C(R^{X1})(R^{X2})C(O)O;
2.37. Compound of Formula II or any of 2.1-2.5, wherein X—Y—Z—R² is:

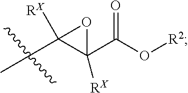

2.38. Compound of Formula II or any of 2.1-2.37, wherein one or more of R^{X}, R^{X1} and R^{X2} are H;
2.39. Compound of Formula II or any of 2.1-2.38, wherein one or more of R^{X}, R^{X1} and R^{X2} are OH;
2.40. Compound of Formula II or any of 2.1-2.39, wherein one or more of R^{X}, R^{X1} and R^{X2} are C₁₋₆alkyl (e.g., methyl or ethyl);
2.41. Compound of Formula II or any of 2.1-2.40, wherein one or more of R^{X}, R^{X1} and R^{X2} are C₃₋₆cycloalkyl (e.g., cyclopropyl);
2.42. Compound of Formula II or any of 2.1-2.41, wherein one or more of R^{X}, R^{X1} and R^{X2} are C₂₋₆alkenyl (e.g., vinyl or allyl), optionally in E- or Z-stereochemistry;
2.43. Compound of Formula II or any of 2.1-2.37, wherein R^{X}, R^{X1} and R^{X2} are each independently selected from H and C₁₋₆alkyl (e.g., methyl or ethyl);
2.44. Compound of Formula II or any of 2.1-2.37, wherein R^{X}, R^{X1} and R^{X2} are each independently selected from H and methyl;
2.45. Compound of Formula II or any of 2.1-2.37, wherein R^{X}, R^{X1} and R^{X2} are each H;
2.46. Compound of Formula II or any of 2.1-2.37, wherein R^{X}, R^{X1} and R^{X2} are each methyl;
2.47. Compound of Formula II or any of 2.1-2.46, wherein R² is H;
2.48. Compound of Formula II or any of 2.1-2.46, wherein R² is OH;
2.49. Compound of Formula II or any of 2.1-2.46, wherein R² is C₁₋₆alkyl;
2.50. Compound 2.49, wherein R² is selected from methyl, ethyl, propyl, isopropyl, n-butyl, sec-butyl, isobutyl, tert-butyl, n-pentyl, isopentyl, neopentyl, 3-pentyl, 3-methyl-2-butyl, n-hexyl, and 2-methyl-3-pentyl;
2.51. Compound of Formula II or any of 2.1-2.46, wherein R² is C₃₋₆cycloalkyl;
2.52. Compound 2.51, wherein R² is cyclopropyl, cyclobutyl, or cyclopentyl;
2.53. Compound of Formula II or any of 2.1-2.46, wherein R² is C₂₋₆alkenyl, optionally in E- or Z-stereochemistry;
2.54. Compound 2.53, wherein R² is vinyl, allyl, crotyl, methallyl, 3,3-dimethylallyl, 1-propen-1-yl, 2-buten-1-yl, 2-buten-2-yl, 3-buten-1-yl, 2-penten-2-yl, or 3-hexen-1-yl;
2.55. Compound of Formula II or any of 2.1-2.46, wherein R² is C₃₋₆cycloalkenyl (e.g., cyclopentenyl, or cyclohexenyl);
2.56. Compound of Formula II or any of 2.1-2.46, wherein R² is C₆₋₁₂bicycloalkyl (e.g., bicyclo[4.2.0]octanyl, bicyclo[4.3.0]nonanyl, bicyclo[4.4.0]decanyl, bicyclo[2.2.1]heptanyl, bicyclo[2.2.2]octanyl, bicyclo[3.2.2]nonanyl);
2.57. Compound of Formula II or any of 2.1-2.46, wherein R² is C₆₋₁₂bicycloalkenyl (e.g., bicyclo[2.2.1]hept-2-en-2-yl, bicyclo[2.2.1]hept-5-en-2-yl, bicyclo[2.2.2]octen-5-en-2-yl, bicyclo[2.2.2]oct-2-en-2-yl);
2.58. Compound of Formula II or any of 2.1-2.46, wherein R² is aryl (e.g., phenyl, naphthyl);
2.59. Compound of Formula II or any of 2.1-2.46, wherein R² is heteroaryl;
2.60. Compound 2.59, wherein R² is 5-membered heteroaryl (e.g., thiophenyl, furanyl, pyrrolyl, oxazolyl, isoxazolyl, pyrazolyl, imidazolyl, thiazolyl, or isothiazolyl);
2.61. Compound 2.59, wherein R² is 6-membered heteroaryl (e.g., pyridinyl, pyrimidinyl, pyrazinyl, or pyridazinyl);
2.62. Compound 2.59, wherein R² is 9- to 12-membered bicyclic heteroaryl (e.g., benzofuranyl, benzothiophenyl, indolyl, indazolyl, benzimidazolyl, benzoxazolyl, benzothiazolyl)
2.63. Compound of Formula II, or any of 2.1-2.62, wherein the compound is a Compound of Formula IIa:

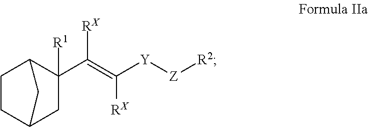

wherein Y is C(R^{X1})(R^{X2})C(R^{X1})(R^{X2}) or a bond, Z is CHO, C(O)O, C(O), C(R^{X1})(R^{X2})O, or C(R^{X1})(R^{X2})OC(O), and R² is H, C₁₋₅alkyl, C₃₋₅cycloalkyl, or C₂₋₅alkenyl;
2.64. Compound 2.63, wherein R^{X} is independently selected from H and C₁₋₆alkyl (e.g., methyl);
2.65. Compound 2.63, wherein R^{X} is independently selected from H and methyl;
2.66. Compound 2.63, wherein R^{X} is H;
2.67. Any one of Compounds 2.63-2.66, wherein R^{X1} and R^{X2} are each independently selected from H and C₁₋₆alkyl (e.g., methyl or ethyl);
2.68. Any one of Compounds 2.63-2.66, wherein R^{X1} and R^{X2} are each independently selected from H, methyl, and ethyl, e.g., R^{X1} and R² are each H;
2.69. Any one of Compounds 2.63-2.66, wherein Y is selected from —CH₂CH(Me)-, —CH(Me)CH₂—, —CH(Me)CH(Me)-, —CH₂C(Me)₂-, —C(Me)₂CH₂—, —CH₂CH(Et)-, —CH(Et)CH₂—;
2.70. Any one of Compounds 2.63-2.66, wherein Y is —CH₂CH(Me)-;
2.71. Any one of Compounds 2.63-2.66, wherein Y is a bond;
2.72. Any one of Compounds 2.63-2.71, wherein Z is CHO;
2.73. Any one of Compounds 2.63-2.71, wherein Z is C(R^{X1})(R^{X2})O, optionally wherein R² is H;
2.74. Compound 2.73, wherein R^{X1} and R^{X2} are each independently selected from H and C₁₋₆alkyl (e.g., methyl);
2.75. Any one of Compounds 2.63-2.71, wherein Z is C(O)O, C(O), or C(R^{X1})(R^{X2})OC(O);
2.76. Compound 2.75, wherein R^{X1} and R^{X2} are each independently selected from H and C₁₋₆alkyl (e.g., methyl);
2.77. Any one of Compounds 2.63-2.76, wherein R² is H;
2.78. Any one of Compounds 2.63-2.76, wherein R² is C₁₋₆alkyl (e.g., methyl, ethyl, propyl, isopropyl);
2.79. Any one of Compounds 2.63-2.76, wherein R² is C₃₋₆cycloalkyl (e.g., cyclopropyl, cyclobutyl);
2.80. Any one of Compounds 2.63-2.76, wherein R² is C₂₋₆alkenyl (e.g., vinyl, allyl, methallyl), optionally in E- or Z-stereochemistry;
2.81. Any one of Compounds 2.63-2.80, wherein Z is C(O)O and R² is C₁₋₆alkyl (e.g., methyl, ethyl, propyl, isopropyl);
2.82. Any one of Compounds 2.63-2.80, wherein Z is C(O)O and R² is C₃₋₆cycloalkyl (e.g., cyclopropyl, cyclobutyl);
2.83. Any one of Compounds 2.63-2.80, wherein Z is C(O)O and R² is C₂₋₆alkenyl (e.g., vinyl, allyl, methallyl), optionally in E- or Z-stereochemistry;
2.84. Any one of Compounds 2.63-2.80, wherein Z is C(O)O and R² is methyl, ethyl, cyclopropyl, propyl, or isopropyl;
2.85. Any one of Compounds 2.63-2.84, wherein Z is CH₂O and R² is H;
2.86. Any one of Compounds 2.63-2.85, wherein R¹ is H or methyl;
2.87. Compound of Formula II, or any of 2.1-2.62, wherein the compound is a Compound of Formula IIb:

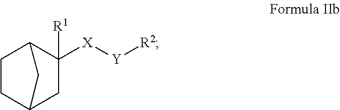

wherein X is C(R^{X1})(R^{X2})O, or C(O)(O), and Y is C(R^{X1})(R^{X2}) or C(O), provided that X is not C(O)O when Y is C(O);
2.88. Compound 2.87, wherein X is C(R^{X1})(R^{X2})O;
2.89. Compound 2.88, wherein R^{X1} and R^{X2} are each independently selected from H, C₁₋₆alkyl (e.g., methyl or ethyl), and C₂₋₅alkenyl (e.g., vinyl, allyl), optionally in E- or Z-stereochemistry;
2.90. Compound 2.89, wherein R^{X1} and R^{X2} are each independently selected from H, methyl, ethyl, and vinyl;
2.91. Compound 2.89, wherein C(R^{X1})(R^{X2})O is CH₂O, CH(Me)O, or CH(CH═CH₂)O;
2.92. Compound 2.87, wherein X is C(O)O;
2.93. Any of Compounds 2.87-2.92, wherein Y is C(R^{X1})(R^{X2});
2.94. Compound 2.93, wherein R^{X1} and R^{X2} are each independently selected from H C₁₋₆alkyl (e.g., methyl or ethyl), and C₂₋₅alkenyl (e.g., vinyl, allyl), optionally in E- or Z-stereochemistry;
2.95. Compound 2.94, wherein C(R^{X1})(R^{X2})O is CH₂O;
2.96. Any of Compounds 2.87-2.92, wherein Y is C(O);
2.97. Any of Compounds 2.87-2.96, wherein R² is H;
2.98. Any one of Compounds 2.87-2.96, wherein R² is C₁₋₆alkyl (e.g., methyl, ethyl, propyl, isopropyl);
2.99. Compound 2.98, wherein R² is selected from methyl, ethyl, propyl, isopropyl, n-butyl, sec-butyl, isobutyl, n-pentyl, 3-pentyl, 3-methyl-2-butyl, n-hexyl, and 2-methyl-3-pentyl;
2.100. Any one of Compounds 2.87-2.96, wherein R² is C₃₋₆cycloalkyl (e.g., cyclopropyl, cyclobutyl);
2.101. Any one of Compounds 2.87-2.96, wherein R² is C₂₋₆alkenyl (e.g., vinyl, allyl, methallyl), optionally in E- or Z-stereochemistry;
2.102. Compound 2.101, wherein R² is vinyl, allyl, crotyl, methallyl, 3,3-dimethylallyl, 1-propen-1-yl, 2-buten-1-yl, 2-buten-2-yl, 3-buten-1-yl, 2-penten-2-yl, or 3-hexen-1-yl;
2.103. Any one of Compounds 2.87-2.96, wherein R² is C₆₋₁₂bicycloalkyl or C₆₋₁₂bicycloalkenyl (e.g., bicyclo[2.2.1]heptanyl, bicyclo[2.2.1]hept-2-en-2-yl, bicyclo[2.2.1]hept-5-en-2-yl);
2.104. Any one of Compounds 2.87-2.96, wherein R² is heteroaryl;
2.105. Compound 2.104, wherein R² is thiophenyl, furanyl, or pyridyl;
2.106. Any one of Compounds 2.87-2.96, wherein R² is aryl (e.g., phenyl);
2.107. Any one of Compounds 2.87-2.106, wherein R¹ is H or methyl;
2.108. Compound of Formula II, or any of 2.1-2.62, wherein the compound is a Compound of Formula IIc:

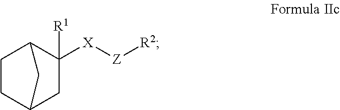

wherein X is C(R^{X1})(R^{X2}) or C(R^{X1})(R^{X2})C(R^{X1})(R^{X2}), and Z is CN, CHO, C(R^{X1})(R^{X2})CN, C(R^{X1})(R^{X2})CHO, C(R^{X1})(R^{X2})O, and C(R^{X1})(R^{X2})C(O)O;
2.109. Compound 2.108, wherein X is C(R^{X1})(R^{X2});
2.110. Compound 2.109, wherein R^{X1} and R^{X2} are each independently selected from H, C₁₋₆alkyl (e.g., methyl or ethyl), and C₂₋₅alkenyl (e.g., vinyl, allyl), optionally in E- or Z-stereochemistry;
2.111. Compound 2.109, wherein R^{X1} and R^{X2} are each independently selected from H, and C₁₋₆alkyl (e.g., methyl or ethyl);
2.112. Compound 2.109, wherein R^{X1} and R^{X2} are each independently selected from H and methyl, e.g., wherein X is CH₂, CH(Me), or CH(Et);
2.113. Compound 2.108, wherein X is C(R^{X1})(R^{X2})C(R^{X1})(R^{X2});
2.114. Compound 2.113, wherein each R^{X1} and R^{X2} are each independently selected from H, C₁₋₆alkyl (e.g., methyl or ethyl), and C₂₋₅alkenyl (e.g., vinyl, allyl), optionally in E- or Z-stereochemistry;
2.115. Compound 2.114, wherein each R^{X1} and R^{X2} are each independently selected from H, and C₁₋₆alkyl (e.g., methyl or ethyl);
2.116. Compound 2.114, wherein each R^{X1} and R^{X2} are each independently selected from H, methyl, and ethyl;
2.117. Compound 2.116, wherein X is CH₂CH₂, CH₂CH(Me), CH₂CH(Et), CH(Me)CH₂, or CH(Et)CH₂;
2.118. Any of Compounds 2.108-2.117, wherein Z is CN and R² is absent;
2.119. Any of Compounds 2.108-2.117, wherein Z is CHO and R² is absent;
2.120. Any of Compounds 2.108-2.117, wherein Z is C(R^{X1})(R^{X2})CN and R² is absent;
2.121. Any of Compounds 2.108-2.117, wherein Z is C(R^{X1})(R^{X2})CHO and R² is absent;
2.122. Any of Compounds 2.108-2.117, wherein Z is C(R^{X1})(R^{X2})C(O)O, and R² is C₁₋₆alkyl (e.g., methyl, ethyl, propyl, isopropyl);
2.123. Any of Compounds 2.108-2.117, wherein Z is C(R^{X1})(R^{X2})C(O)O, and R² is C₂₋₆alkenyl (e.g., vinyl, allyl, methallyl), optionally in E- or Z-stereochemistry;
2.124. Any of Compounds 2.108-2.117, wherein Z is C(R^{X1})(R^{X2})O, and R² is H;
2.125. Compound 2.120, 2.121, 2.122, 2.123, or 2.124, wherein R^{X1} and R^{X2} are each independently selected from H and methyl, e.g., wherein the C(R^{X1})(R^{X2}) of Z is CH₂ or CH(Me);
2.126. Any one of Compounds 2.108-2.125, wherein R¹ is H or methyl;
2.127. Compound of Formula II, wherein the compound is a Compound of Formula IId:

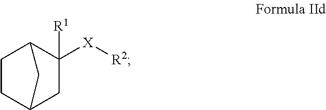

wherein X is C(R^{X1})(R^{X2}), C(R^{X1})(R^{X2})O, C(O) or C(O)(O), or
wherein X—R² is:

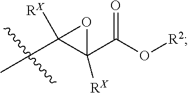

2.128. Compound of 2.127, wherein X is C(R^{X1})(R^{X2});
2.129. Compound of 2.127, wherein X is C(R^{X1})(R^{X2})O;
2.130. Compound 2.128 or 2.129, wherein R^{X1} and R² are each independently selected from H, C₁₋₆alkyl (e.g., methyl or ethyl), and C₂₋₅alkenyl (e.g., vinyl, allyl), optionally in E- or Z-stereochemistry;
2.131. Compound 2.130, wherein R^{X1} and R^{X2} are each independently selected from H, methyl, ethyl, and vinyl;
2.132. Compound 2.130, wherein X is CH₂O, CH(Me)O, or CH(CH═CH₂)O;
2.133. Compound 2.130, wherein X is CH₂, CH(Me), or CH(CH═CH₂);
2.134. Compound 2.127, wherein X is C(O);
2.135. Compound 2.127, wherein X is C(O)O;
2.136. Compound 2.127, wherein X—R² is:

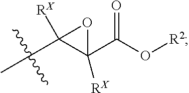

2.137. Any of Compounds 2.127-2.136, wherein R² is C₁₋₆alkyl (e.g., methyl, ethyl, propyl, isopropyl);
2.138. Compound 2.128, wherein R² is selected from methyl, ethyl, propyl, isopropyl, n-butyl, sec-butyl, isobutyl, n-pentyl, 3-pentyl, 3-methyl-2-butyl, n-hexyl, and 2-methyl-3-pentyl;
2.139. Any one of Compounds 2.127-2.136, wherein R² is C₃₋₆cycloalkyl (e.g., cyclopropyl, cyclobutyl);
2.140. Any one of Compounds 2.127-2.136, wherein R² is C₂₋₆alkenyl (e.g., vinyl, allyl, methallyl), optionally in E- or Z-stereochemistry;
2.141. Compound 2.140, wherein R² is vinyl, allyl, crotyl, methallyl, 3,3-dimethylallyl, 1-propen-1-yl, 2-buten-1-yl, 2-buten-2-yl, 3-buten-1-yl, 2-penten-2-yl, or 3-hexen-1-yl;
2.142. Any one of Compounds 2.127-2.136, wherein R² is C₆₋₁₂bicycloalkyl or C₆₋₁₂bicycloalkenyl (e.g., bicyclo[2.2.1]heptanyl, bicyclo[2.2.1]hept-2-en-2-yl, bicyclo[2.2.1]hept-5-en-2-yl);
2.143. Any one of Compounds 2.127-2.136, wherein R² is heteroaryl;
2.144. Compound 2.143, wherein R² is 5-membered heteroaryl (e.g., thiophenyl, furanyl, pyrrolyl, oxazolyl, isoxazolyl, pyrazolyl, imidazolyl, thiazolyl, or isothiazolyl);
2.145. Any one of Compounds 2.127-2.136, wherein R² is aryl (e.g., phenyl);
2.146. Any one of Compounds 2.127-2.145, wherein R¹ is H or methyl;
2.147. Compound of Formula II, or any of 2.1-2.146, wherein the compound of Formula II is selected from any of the following:

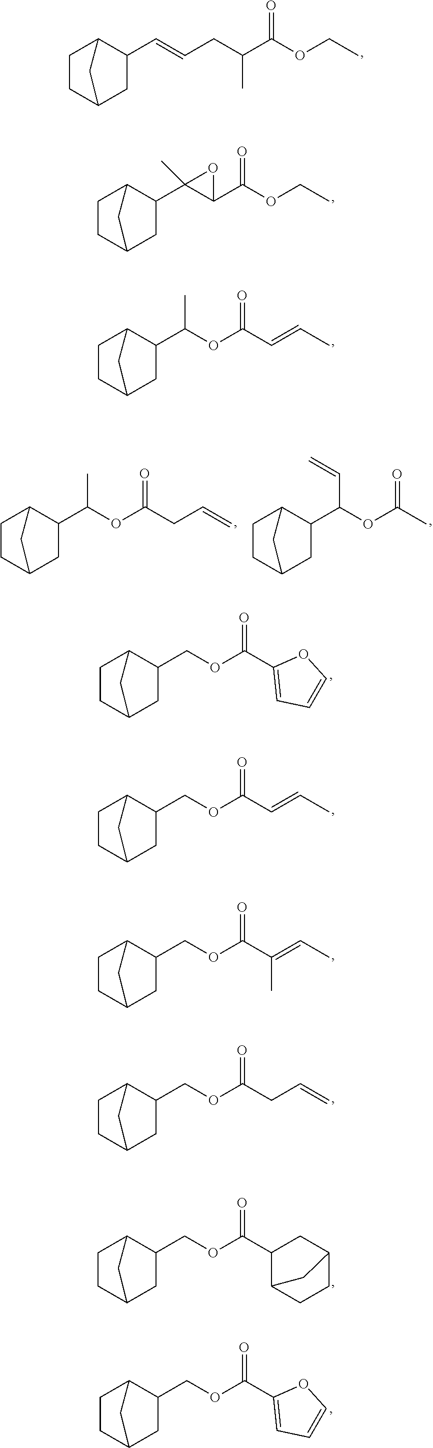

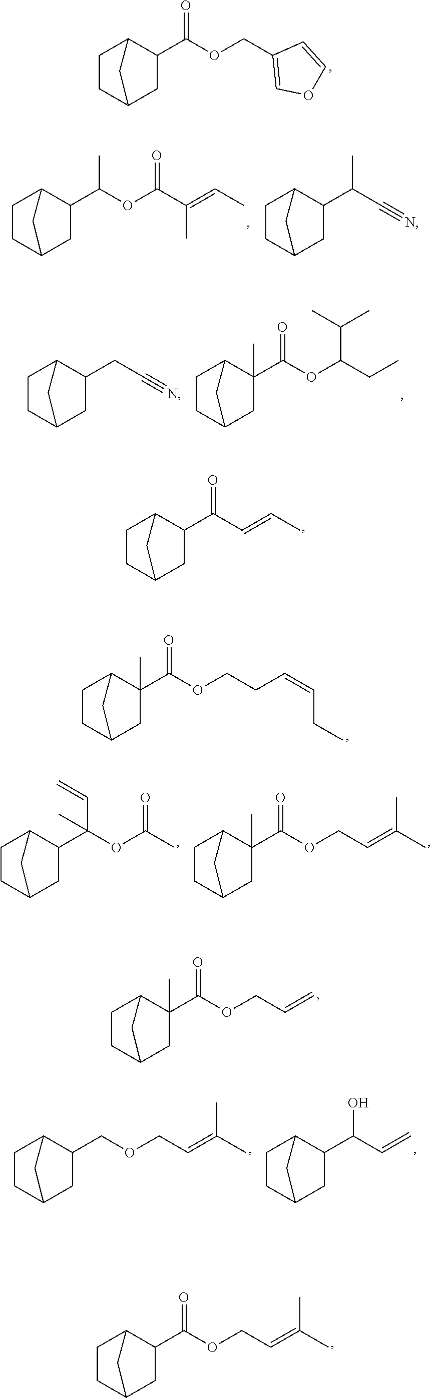

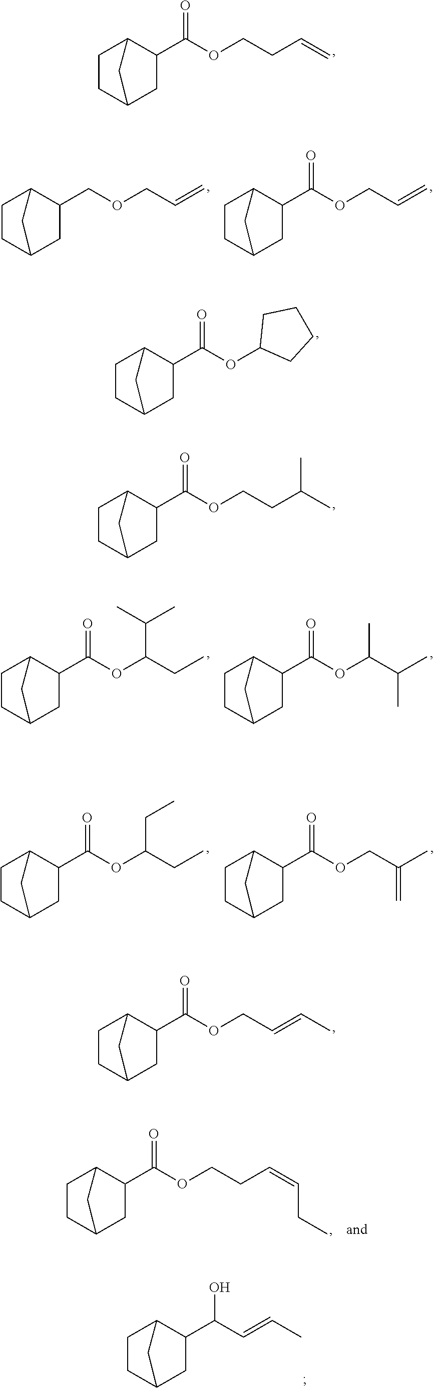

2.148. Compound of Formula 11, or any of 2.1-2.146, wherein the compound of Formula 11 is selected from any of the following:

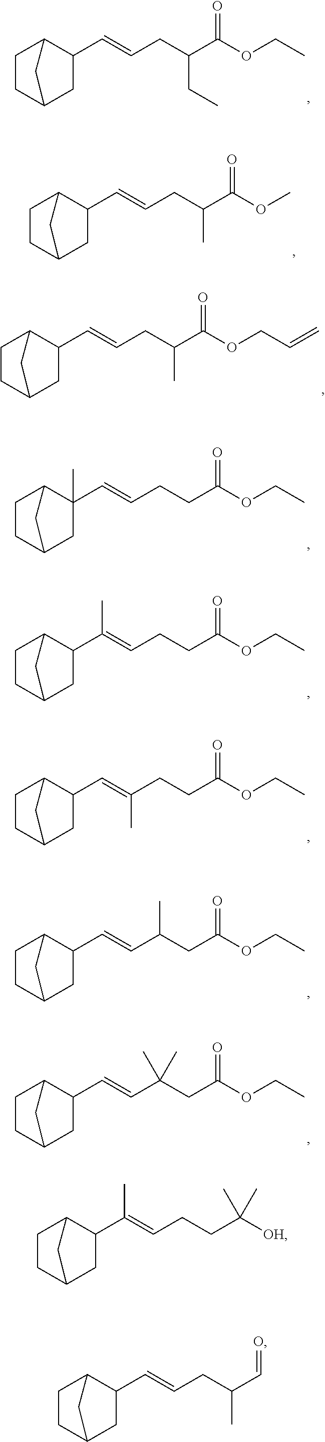

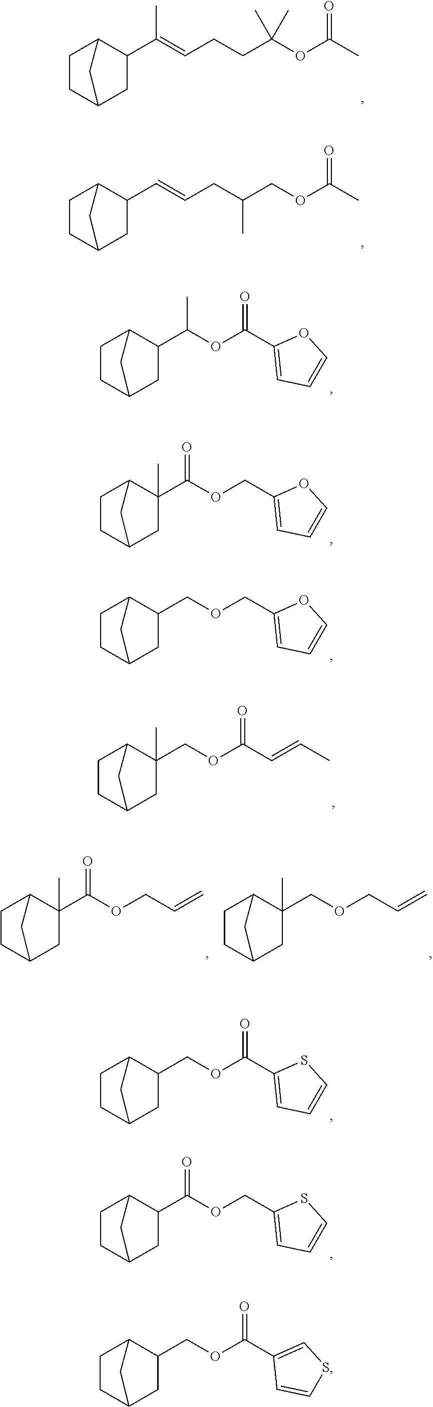

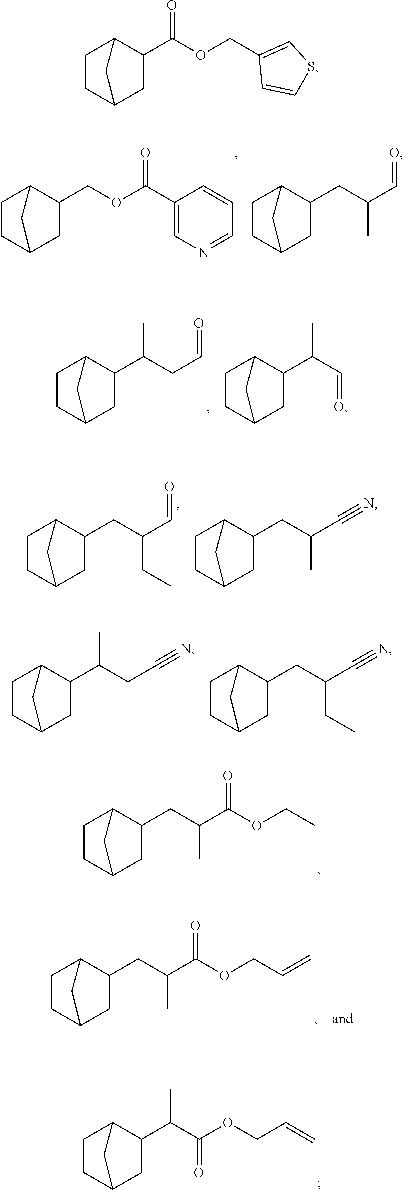

2.149. Compound of Formula 11, or any of 2.1-2.62, wherein the compound is a Compound of Formula Ile:

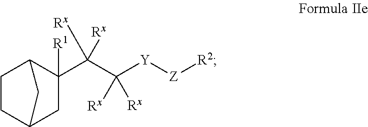

wherein Y is C(R^{X1})(R^{X2})C(R^{X1})(R^{X2}) or a bond, Z is CHO, C(O)O, C(O), C(R^{X1})(R^{X2})O, or C(R^{X1})(R^{X2})OC(O), and R² is H, C₁₋₅alkyl, C₃₋₅cycloalkyl, or C₂₋₅alkenyl;
2.150. Compound 2.149, wherein R^{X} is independently selected from H and C₁₋₆alkyl (e.g., methyl);
2.151. Compound 2.149, wherein R^{X} is independently selected from H and methyl;
2.152. Compound 2.149, wherein R^{X} is H;
2.153. Any one of Compounds 2.149-2.152, wherein R^{X1} and R^{X2} are each independently selected from H and C₁₋₆alkyl (e.g., methyl or ethyl);
2.154. Any one of Compounds 2.149-2.152, wherein R^{X1} and R^{X2} are each independently selected from H, methyl, and ethyl, e.g., R^{X1} and R^{X2} are each H;
2.155. Any one of Compounds 2.149-2.152, wherein Y is selected from —CH₂CH(Me)-, —CH(Me)CH₂—, —CH(Me)CH(Me)-, —CH₂C(Me)₂-, —C(Me)₂CH₂—, —CH₂CH(Et)-, —CH(Et)CH₂—;
2.156. Any one of Compounds 2.149-2.152, wherein Y is —CH₂CH(Me)-;
2.157. Any one of Compounds 2.149-2.152, wherein Y is a bond;
2.158. Any one of Compounds 2.149-2.157, wherein Z is CHO;
2.159. Any one of Compounds 2.149-2.157, wherein Z is C(R^{X1})(R^{X2})O, optionally wherein R² is H;
2.160. Compound 2.159, wherein R^{X1} and R^{X2} are each independently selected from H and C₁₋₆alkyl (e.g., methyl);
2.161. Any one of Compounds 2.149-2.157, wherein Z is C(O)O, C(O), or C(R^{X1})(R^{X2})OC(O);
2.162. Compound 2.161, wherein R^{X1} and R^{X2} are each independently selected from H and C₁₋₆alkyl (e.g., methyl);
2.163. Any one of Compounds 2.149-2.162, wherein R² is H;
2.164. Any one of Compounds 2.149-2.162, wherein R² is C₁₋₆alkyl (e.g., methyl, ethyl, propyl, isopropyl);
2.165. Any one of Compounds 2.149-2.162, wherein R² is C₃₋₆cycloalkyl (e.g., cyclopropyl, cyclobutyl);
2.166. Any one of Compounds 2.149-2.162, wherein R² is C₂₋₆alkenyl (e.g., vinyl, allyl, methallyl), optionally in E- or Z-stereochemistry;
2.167. Any one of Compounds 2.149-2.166, wherein Z is C(O)O and R² is C₁₋₆alkyl (e.g., methyl, ethyl, propyl, isopropyl);
2.168. Any one of Compounds 2.149-2.166, wherein Z is C(O)O and R² is C₃₋₆cycloalkyl (e.g., cyclopropyl, cyclobutyl);
2.169. Any one of Compounds 2.149-2.166, wherein Z is C(O)O and R² is C₂₋₆alkenyl (e.g., vinyl, allyl, methallyl), optionally in E- or Z-stereochemistry;
2.170. Any one of Compounds 2.149-2.166, wherein Z is C(O)O and R² is methyl, ethyl, cyclopropyl, propyl, or isopropyl;
2.171. Any one of Compounds 2.149-2.170, wherein Z is CH₂O and R² is H;
2.172. Any one of Compounds 2.149-2.171, wherein R¹ is H or methyl;
2.173. Compound of Formula II, or any of Compounds 2.1-2.144 or 2.149-2.172, wherein the compound of Formula II is selected from any of the following:

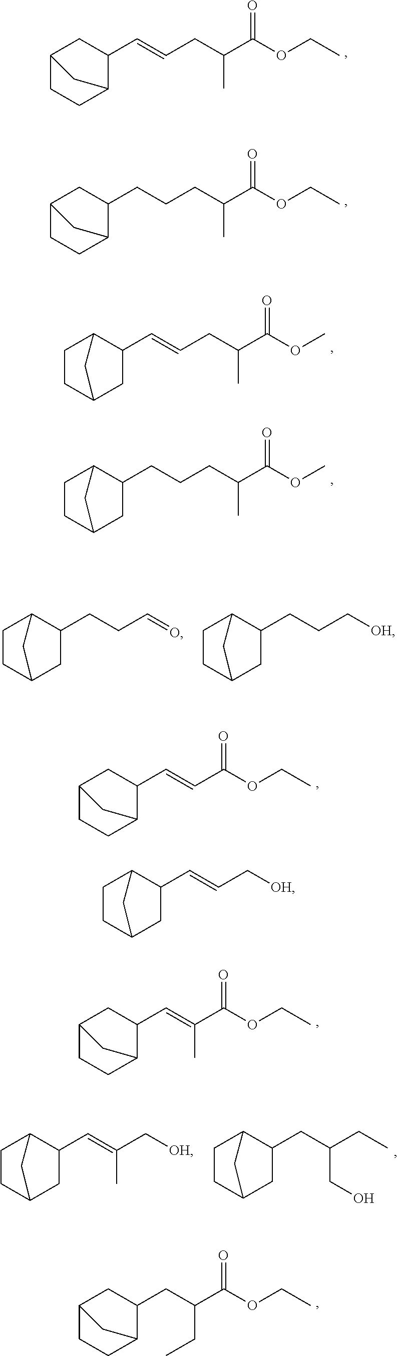

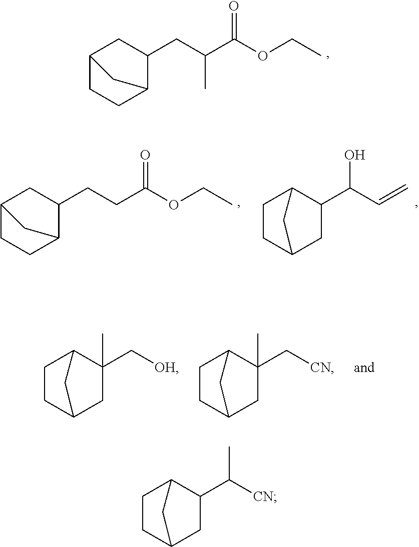

2.174. Compound of Formula II, or any of 2.1-2.62, wherein the compound is a Compound of Formula IIf:

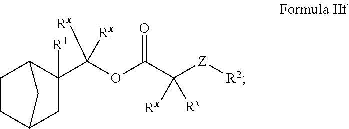

wherein R^{X} is independently selected from H, C₁₋₆alkyl, C₂₋₆alkenyl, and C₃₋₆cycloalkyl, Z is CN, CHO, C(O)O, or C(O), and R² is H, C₁₋₅alkyl, C₃₋₅cycloalkyl, or C₂₋₅alkenyl;
provided that when Z is CN or CHO, then R² is absent;
2.175. Compound 2.174, wherein R^{X} is independently selected from H and C₁₋₆alkyl (e.g., methyl or ethyl);
2.176. Compound 2.174, wherein R^{X} is independently selected from H, methyl, and ethyl;
2.177. Compound 2.174, wherein at least one R^{X} is independently C₁₋₆alkyl (e.g., methyl or ethyl);
2.178. Compound 2.174, wherein at least two R^{X} are independently C₁₋₆alkyl (e.g., methyl or ethyl), optionally wherein there are two C₁₋₆alkyl on different carbon atoms;
2.179. Any one of compounds 2.174-2.178, wherein one of the two C(R^{X}) moieties is a CH₂;
2.180. Any one of compounds 2.174-1.179, wherein at least one R^{X} is independently C₂₋₆alkenyl (e.g., vinyl);
2.181. Any one of compounds 2.174-1.180, wherein at least one R^{X} is independently C₃₋₆cycloalkyl (e.g., cyclopropyl);
2.182. Any one of compounds 2.174-1.179, wherein R^{X} is independently selected from H, methyl, ethyl, vinyl, and cyclopropyl;
2.183. Any one of compounds 2.174-2.182, wherein the Compound of Formula IIf is a Compound of Formula IIf-1:

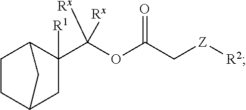

2.184. Any one of compounds 2.174-2.182, wherein the Compound of Formula IIf is a Compound of Formula IIf-2:

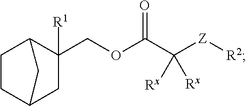

2.185. Any one of compounds 2.174-2.182, wherein the Compound of Formula IIf is a Compound of Formula IIf-3:

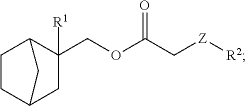

2.186. Any one of Compounds 2.174-2.185, wherein Z is CHO;
2.187. Any one of Compounds 2.174-2.185, wherein Z is CN;
2.188. Any one of Compounds 2.174-2.185, wherein Z is C(O)O;
2.189. Any one of Compounds 2.174-2.185, wherein Z is C(O);
2.190. Any one of Compounds 2.174-2.189, wherein R² is H;
2.191. Any one of Compounds 2.174-2.189, wherein R² is C₁₋₆alkyl (e.g., methyl, ethyl, propyl, isopropyl);
2.192. Any one of Compounds 2.174-2.189, wherein R² is C₃₋₆cycloalkyl (e.g., cyclopropyl, cyclobutyl);
2.193. Any one of Compounds 2.174-2.189, wherein R² is C₂₋₆alkenyl (e.g., vinyl, allyl, methallyl), optionally in E- or Z-stereochemistry;
2.194. Any one of Compounds 2.174-2.185, wherein Z is C(O)O and R² is C₁₋₆alkyl (e.g., methyl, ethyl, propyl, isopropyl);
2.195. Any one of Compounds 2.174-2.185, wherein Z is C(O)O and R² is C₃₋₆cycloalkyl (e.g., cyclopropyl, cyclobutyl);
2.196. Any one of Compounds 2.174-2.185, wherein Z is C(O)O and R² is C₂₋₆alkenyl (e.g., vinyl, allyl, methallyl), optionally in E- or Z-stereochemistry;
2.197. Any one of Compounds 2.174-2.185, wherein Z is C(O)O and R² is methyl, ethyl, cyclopropyl, propyl, or isopropyl;
2.198. Any one of Compounds 2.174-2.185, wherein Z is C(O) and R² is C₁₋₆alkyl (e.g., methyl, ethyl, propyl, isopropyl);
2.199. Any one of Compounds 2.174-2.185, wherein Z is C(O) and R² is C₃₋₆cycloalkyl (e.g., cyclopropyl, cyclobutyl);
2.200. Any one of Compounds 2.174-2.185, wherein Z is C(O) and R² is C₂₋₆alkenyl (e.g., vinyl, allyl, methallyl), optionally in E- or Z-stereochemistry;
2.201. Any one of Compounds 2.174-2.185, wherein Z is C(O) and R² is methyl, ethyl, cyclopropyl, propyl, or isopropyl;
2.202. Any one of Compounds 2.174-1.201, wherein R¹ is H;
2.203. Any one of Compounds 2.174-1.201, wherein Ri is methyl;
2.204. Compound of Formula II, or any of Compounds 2.1-2.144 or 2.174-2.203, wherein the compound of Formula II is selected from any of the following:

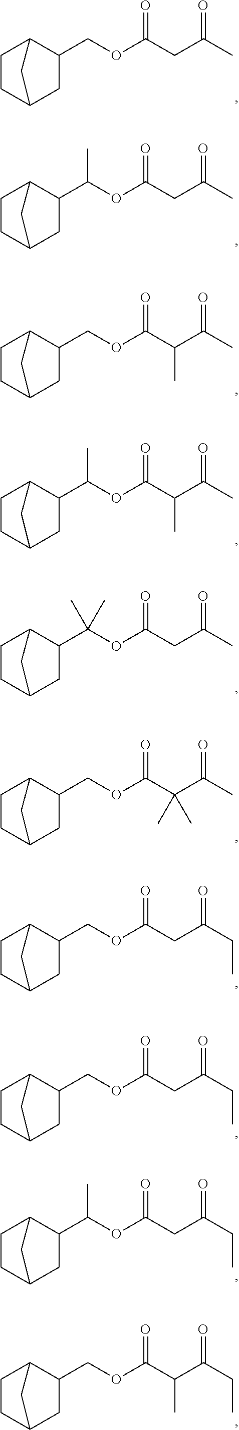

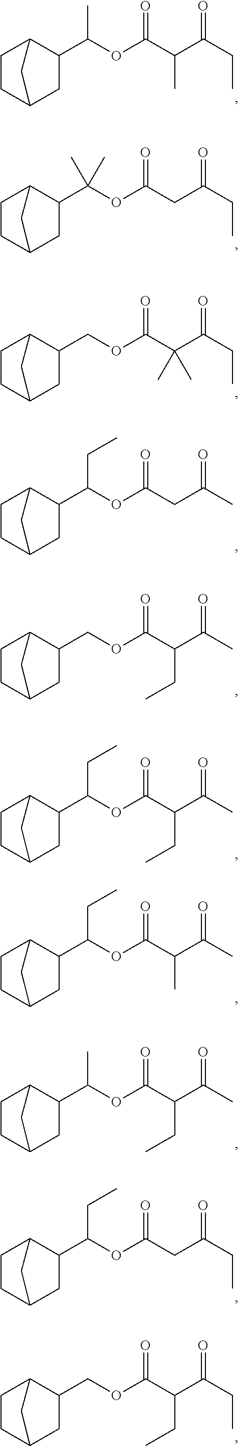

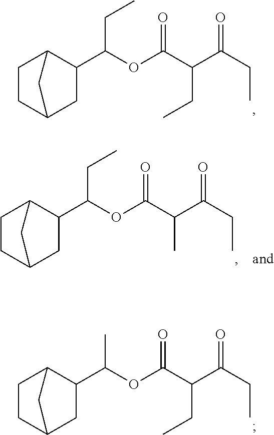 [SMILES: CCC(=O)C(CC)C(=O)OC(CC)C1CC2CCC1C2 ; CCC(=O)C(C)C(=O)OC(CC)C1CC2CCC1C2 ; CCC(=O)C(CC)C(=O)OC(C)C1CC2CCC1C2]

2.205. Any preceding compound, wherein the compound has endo stereochemistry about the norbornane ring;
2.206. Any preceding compound, wherein the compound has exo stereochemistry about the norbornane ring;
2.207. Any preceding compound, wherein the compound has a molecular weight selected from the range of to 110 to 600, or 110 to 500, or 110 to 450, or 110 to 400, or 110 to 350, or 110 to 300, or 110 to 250, or 110 to 200;
2.208. Any preceding compound, wherein the compound has a pleasing taste and/or aroma, e.g., as judged by a trained flavor or fragrance chemist or master perfumer (e.g., toasted coconut or other nutty aromas and/or tastes).

In some embodiments of any of the aspects disclosed herein, for example the first aspect, the compound of Formula I or the Compound of Formula II does not include one or more of the following compounds (“Selected Compounds”):
2-(Bicyclo[2.2.1]hept-5-en-2-yl)acetonitrile,
Bicyclo [2.2.1]heptane-2-acetonitrile,
α-Methylbicyclo[2.2.1]hept-5-ene-2-acetonitrile,
α-Methylbicyclo[2.2.1]heptane-2-acetonitrile,
α,α-Dimethylbicyclo[2.2.1]hept-5-ene-2-acetonitrile,
α,α-Dimethylbicyclo[2.2.1]heptane-2-acetonitrile,
2-Methylbicyclo[2.2.1]hept-5-ene-2-acetic acid,
1-(2-Methylbicyclo[2.2.1]hept-2-yl)-2-propanone,
2-Methylbicyclo[2.2.1]heptane-2-acetic acid,
2-(2-Methylbicyclo[2.2.1]hept-2-yl)-3-pentanone,
2-Propen-1-yl 2-methylbicyclo[2.2.1]hept-5-ene-2-acetate
3,3-Dimethyl-1-(2-methylbicyclo[2.2.1]hept-2-yl)-2-butanone,
Bicyclo[2.2.1]hept-5-ene-2-acetaldehyde,
1-Bicyclo[2.2.1]hept-5-en-2-yl-2-propanone,
Bicyclo[2.2.1]hept-5-ene-2-acetic acid,
α-Methylbicyclo[2.2.1]heptane-2-acetaldehyde,
1-Bicyclo[2.2.1]hept-2-yl-2-propanone,
Bicyclo[2.2.1]heptane-2-acetic acid,
1-Bicyclo[2.2.1]hept-2-yl-3-buten-2-one,
Methyl bicyclo[2.2.1]hept-5-ene-2-acetate,
α-Methylbicyclo[2.2.1]hept-5-ene-2-acetic acid,
α,α-Dimethylbicyclo[2.2.1]heptane-2-acetaldehyde,
1-Bicyclo[2.2.1]hept-2-yl-2-butanone,
3-Bicyclo[2.2.1]hept-2-yl-2-butanone,
α-Hydroxybicyclo[2.2.1]hept-5-ene-2-acetic acid,
1-Bicyclo[2.2.1]hept-2-yl-1-hydroxy-2-propanone,
α-Hydroxy-a-methylbicyclo[2.2.1]heptane-2-acetaldehyde,
α-Hydroxybicyclo[2.2.1]heptane-2-propanal,
Methyl bicyclo[2.2.1]heptane-2-acetate,
α-Methylbicyclo[2.2.1]heptane-2-acetic acid,
α-Cyclopropylbicyclo[2.2.1]hept-5-ene-2-acetaldehyde,
α-Cyclopropylbicyclo[2.2.1]heptane-2-acetaldehyde,
1-Bicyclo[2.2.1]hept-5-en-2-yl-2-pentanone,
1-Bicyclo[2.2.1]hept-2-yl-4-penten-2-one
2-Bicyclo[2.2.1]hept-2-yl-1-cyclopropylethanone,
1-Bicyclo[2.2.1]hept-2-yl-3-methyl-3-buten-2-one,
1-Bicyclo[2.2.1]hept-2-yl-3-penten-2-one,
1-Bicyclo[2.2.1]hept-5-en-2-yl-1-hydroxy-2-butanone,
4-Bicyclo[2.2.1]hept-5-en-2-yl-3-hydroxy-2-butanone,
Ethyl bicyclo[2.2.1]hept-5-ene-2-acetate,
α-Ethylbicyclo[2.2.1]hept-5-ene-2-acetic acid,
α,α-Dimethylbicyclo[2.2.1]hept-5-ene-2-acetic acid,
1-Bicyclo[2.2.1]hept-2-yl-1-hydroxy-3-buten-2-one,
Ethenyl bicyclo[2.2.1]heptane-2-acetate,
α-Ethyl-α-methylbicyclo[2.2.1]heptane-2-acetaldehyde,
3-Bicyclo[2.2.1]hept-2-yl-2-pentanone,
1-Bicyclo[2.2.1]hept-2-yl-2-pentanone,
1-Bicyclo[2.2.1]hept-2-yl-3-methyl-2-butanone,
2-Bicyclo[2.2.1]hept-2-yl-3-pentanone,
α-Hydroxybicyclo[2.2.1]hept-5-ene-2-propanoic acid,
1-Bicyclo[2.2.1]hept-2-yl-1-hydroxy-2-butanone,
α-Ethoxybicyclo[2.2.1]heptane-2-acetaldehyde,
3-Bicyclo[2.2.1]hept-2-yl-3-hydroxy-2-butanone,
Methyl α-methylbicyclo[2.2.1]heptane-2-acetate,
Ethyl bicyclo[2.2.1]heptane-2-acetate,
α-Ethyl bicyclo[2.2.1]heptane-2-acetic acid,
α,α-Dimethylbicyclo[2.2.1]heptane-2-acetic acid,
Methyl α-hydroxybicyclo[2.2.1]heptane-2-acetate
α-Hydroxybicyclo[2.2.1]heptane-2-propanoic acid
α-Hydroxy-α-methylbicyclo [2.2.1]heptane-2-acetic acid,
1-Bicyclo[2.2.1]hept-5-en-2-yl-5-hexen-2-one,
2-Propen-1-yl bicyclo[2.2.1]hept-5-ene-2-acetate,
α-Cyclopropylbicyclo[2.2.1]hept-5-ene-2-acetic acid,
α-2-Propen-1-ylbicyclo[2.2.1]hept-5-ene-2-acetic acid,
1-Bicyclo[2.2.1]hept-5-en-2-yl-2-hexanone
α-Cyclobutylbicyclo[2.2.1]heptane-2-acetaldehyde,
1-Bicyclo[2.2.1]hept-2-yl-5-hexen-2-one,
2-Bicyclo[2.2.1]hept-2-yl-1-cyclobutylethanone,
1-Bicyclo[2.2.1]hept-2-yl-3-hexen-2-one,
1-Bicyclo[2.2.1]hept-2-yl-4-methyl-4-penten-2-one,
1-Bicyclo[2.2.1]hept-2-yl-4-methyl-3-penten-2-one,
1-Bicyclo[2.2.1]hept-5-en-2-yl-1-hydroxy-3-methyl-2-butanone,
1-Bicyclo[2.2.1]hept-5-en-2-yl-1-hydroxy-2-pentanone,
α-(1-Methylethyl)bicyclo[2.2.1]hept-5-ene-2-acetic acid,
2-Bicyclo[2.2.1]hept-2-yl-1-cyclopropyl-2-hydroxyethanone
1-Bicyclo[2.2.1]hept-2-yl-1-hydroxy-4-penten-2-one,
4-Bicyclo[2.2.1]hept-2-yl-4-hydroxy-1-penten-3-one.
Bicyclo[2.2.1]heptane-2-acetic acid, 2-propenyl ester,
1-Bicyclo[2.2.1]hept-2-yl-1-hydroxy-3-penten-2-one,
α-2-Propen-1-ylbicyclo[2.2.1]heptane-2-acetic acid,
α-Cyclopropylbicyclo[2.2.1]heptane-2-acetic acid,
3-Bicyclo[2.2.1]hept-2-yl-4-methyl-2-pentanone,
1-Bicyclo[2.2.1]hept-2-yl-2-hexanone,
2-Bicyclo[2.2.1]hept-2-yl-4-methyl-3-pentanone,
2-Bicyclo[2.2.1]hept-2-yl-3-hexanone,
1-Bicyclo[2.2.1]hept-2-yl-4-methyl-2-pentanone,
1-Bicyclo[2.2.1]hept-2-yl-3-methyl-2-pentanone,
Methyl α-oxobicyclo[2.2.1]heptane-2-propanoate
1-Bicyclo[2.2.1]hept-2-yl-1-hydroxy-3-methyl-2-butanone,
1-Bicyclo[2.2.1]hept-2-yl-1-hydroxy-2-pentanone,
2-Bicyclo[2.2.1]hept-2-yl-2-hydroxy-3-pentanone,
Propyl bicyclo[2.2.1]heptane-2-acetate,
Isopropyl bicyclo[2.2.1]heptane-2-acetate,
Ethyl α-methylbicyclo[2.2.1]heptane-2-acetate,
α-Ethyl-α-methylbicyclo[2.2.1]heptane-2-acetic acid,
α-Propylbicyclo[2.2.1]heptane-2-acetic acid,
Ethyl α-hydroxybicyclo[2.2.1]heptane-2-acetate,
Methyl α-hydroxybicyclo[2.2.1]heptane-2-propanoate,
α-Hydroxy-β-methylbicyclo[2.2.1]heptane-2-propanoic acid,
Bicyclo[2.2.1]hept-5-ene-2-propanenitrile,
Bicyclo[2.2.1]heptane-2-propanenitrile,
β-Oxobicyclo[2.2.1]hept-5-ene-2-propanenitrile,
α-Hydroxybicyclo[2.2.1]hept-5-ene-2-propanenitrile,
β-Hydroxybicyclo[2.2.1]hept-5-ene-2-propanenitrile,
β-Oxobicyclo[2.2.1]heptane-2-propanenitrile,
α-Hydroxybicyclo[2.2.1]heptane-2-propanenitrile,
P-Hydroxybicyclo[2.2.1]heptane-2-propanenitrile,
α-Methyl-β-oxobicyclo[2.2.1]heptane-2-propanenitrile,
α,α-Dimethylbicyclo[2.2.1]heptane-2-propanenitrile,
β-Hydroxy-α-methylbicyclo[2.2.1]heptane-2-propanenitrile,
β-Hydroxy-β-methylbicyclo[2.2.1]heptane-2-propanenitrile,
α-Ethyl-β-oxobicyclo[2.2.1]heptane-2-propanenitrile,
α-Ethyl-β-hydroxybicyclo[2.2.1]heptane-2-propanenitrile,
α-Ethyl-α-methylbicyclo[2.2.1]heptane-2-propanenitrile,
β-Hydroxy-α,α-dimethylbicyclo[2.2.1]heptane-2-propanenitrile,
β-Hydroxy-α,β-dimethylbicyclo[2.2.1]heptane-2-propanenitrile,
α-Cyclopropyl-β-oxobicyclo[2.2.1]heptane-2-propanenitrile,
α-Isopropyl-β-oxobicyclo[2.2.1]heptane-2-propanenitrile,
β-Oxo-α-propylbicyclo[2.2.1]heptane-2-propanenitrile,
β-Hydroxy-α-propylbicyclo[2.2.1]heptane-2-propanenitrile,
α-Ethyl-β-hydroxy-β-methylbicyclo[2.2.1]heptane-2-propanenitrile,
β-Hydroxy-α,α,β-trimethylbicyclo[2.2.1]heptane-2-propanenitrile,
α-Isobutyl-β-oxobicyclo[2.2.1]heptane-2-propanenitrile,
α-Ethyl-β-hydroxy-α,β-dimethylbicyclo[2.2.1]heptane-2-propanenitrile,
β-Hydroxy-β-methyl-α-propylbicyclo[2.2.1]heptane-2-propanenitrile,
3-Bicyclo[2.2.1]hept-5-en-2-yl-2-propenoic acid,
Bicyclo[2.2.1]hept-5-ene-2-propanoic acid,
3-Bicyclo[2.2.1]hept-2-yl-2-propenoic acid,
Bicyclo[2.2.1]heptane-2-propanoic acid,
Methyl 3-bicyclo[2.2.1]hept-5-en-2-yl-2-propenoate,
α-Oxobicyclo[2.2.1]hept-5-ene-2-propanoic acid,
α-Methylbicyclo[2.2.1]hept-5-ene-2-propanoic acid,
β-Methylbicyclo[2.2.1]hept-5-ene-2-propanoic acid,
3-Bicyclo[2.2.1]hept-2-yl-2-butenoic acid,
3-Bicyclo[2.2.1]hept-2-yl-2-methyl-2-propenoic acid,
Methyl bicyclo[2.2.1]hept-5-ene-2-propanoate,
Methyl 3-bicyclo[2.2.1]hept-2-yl-2-propenoate,
Methyl 3-(bicyclo[2.2.1]hept-2-ylidene)propanoate,
03-Hydroxybicyclo[2.2.1]hept-5-ene-2-propanoic acid,
α-Oxobicyclo[2.2.1]heptane-2-propanoic acid,
β-Methylbicyclo[2.2.1]heptane-2-propanoic acid,
α-Methylbicyclo[2.2.1]heptane-2-propanoic acid,
Methyl bicyclo[2.2.1]heptane-2-propanoate,
β-Hydroxybicyclo[2.2.1]heptane-2-propanoic acid,
Ethyl 3-bicyclo[2.2.1]hept-5-en-2-yl-2-propenoate,
Methyl 3-bicyclo[2.2.1]hept-5-en-2-yl-2-methyl-2-propenoate,
Methyl β-oxobicyclo[2.2.1]hept-5-ene-2-propanoate,
3-Bicyclo[2.2.1]hept-2-yl-2-methyl-2-butenoic acid,
α,α-Dimethylbicyclo[2.2.1]hept-5-ene-2-propanoic acid,
β,β-Dimethylbicyclo[2.2.1]hept-5-ene-2-propanoic acid,
2-(Bicyclo[2.2.1]hept-2-ylmethylene)butanoic acid,
Ethyl bicyclo[2.2.1]hept-5-ene-2-propanoate,
Methyl 3-bicyclo[2.2.1]hept-2-yl-2-butenoate,
Ethyl 3-bicyclo[2.2.1]hept-2-yl-2-propenoate,
Methyl 3-bicyclo[2.2.1]hept-2-yl-2-methyl-2-propenoate,
β-Hydroxy-β-methylbicyclo[2.2.1]hept-5-ene-2-propanoic acid,
α-Hydroxy-α-methylbicyclo[2.2.1]hept-5-ene-2-propanoic acid,
Methyl α-hydroxybicyclo[2.2.1]hept-5-ene-2-propanoate,
Methyl β-hydroxybicyclo[2.2.1]hept-5-ene-2-propanoate,
Methyl β-oxobicyclo[2.2.1]heptane-2-propanoate,
α,β-Dimethylbicyclo[2.2.1]heptane-2-propanoic acid,
α-Ethylbicyclo[2.2.1]heptane-2-propanoic acid,
β,β-Dimethylbicyclo[2.2.1]heptane-2-propanoic acid,
α,α-Dimethylbicyclo[2.2.1]heptane-2-propanoic acid,
Ethyl bicyclo[2.2.1]heptane-2-propanoate,
α-Hydroxy-β-oxobicyclo[2.2.1]heptane-2-propanoic acid,
β-Hydroxy-β-methylbicyclo[2.2.1]heptane-2-propanoic acid,
α-Hydroxy-α-methylbicyclo[2.2.1]heptane-2-propanoic acid,
Methyl β-hydroxybicyclo[2.2.1]heptane-2-propanoate,
Propyl 3-bicyclo[2.2.1]hept-5-en-2-yl-2-propenoate,
2-Propen-1-yl 3-bicyclo[2.2.1]hept-2-yl-2-propenoate,
Ethyl β-oxobicyclo[2.2.1]hept-5-ene-2-propanoate,
3-Bicyclo[2.2.1]hept-2-yl-2-ethyl-2-butenoic acid,
α-Ethenyl-α-methylbicyclo[2.2.1]heptane-2-propanoic acid,
α-2-Propen-1-ylbicyclo[2.2.1]heptane-2-propanoic acid,
O-Cyclopropylbicyclo[2.2.1]heptane-2-propanoic acid,
α-Cyclopropylbicyclo[2.2.1]heptane-2-propanoic acid,
Ethyl α-methylbicyclo[2.2.1]hept-5-ene-2-propanoate,
Methyl β,β-dimethylbicyclo[2.2.1]hept-5-ene-2-propanoate,
Ethyl 3-bicyclo[2.2.1]hept-2-yl-2-butenoate,
Propyl 3-bicyclo[2.2.1]hept-2-yl-2-propenoate,
Ethyl 3-bicyclo[2.2.1]hept-2-yl-2-methyl-2-propenoate,
β-Hydroxy-α,α-dimethylbicyclo[2.2.1]hept-5-ene-2-propanoic acid,
Ethyl β-oxobicyclo[2.2.1]heptane-2-propanoate,
Methyl α-methyl-β-oxobicyclo[2.2.1]heptane-2-propanoate,
Ethyl α-oxobicyclo[2.2.1]heptane-2-propanoate,
α-Ethyl-β-methylbicyclo[2.2.1]heptane-2-propanoic acid,
α-Isopropylbicyclo[2.2.1]heptane-2-propanoic acid,
α-Propylbicyclo[2.2.1]heptane-2-propanoic acid,
α-Ethyl-α-methylbicyclo[2.2.1]heptane-2-propanoic acid,
Methyl α-hydroxy-β-oxobicyclo[2.2.1]heptane-2-propanoate,
β-Hydroxy-α,α-dimethylbicyclo[2.2.1]heptane-2-propanoic acid,
α-Hydroxy-α,β-dimethylbicyclo[2.2.1]heptane-2-propanoic acid,
Ethyl α-hydroxybicyclo[2.2.1]heptane-2-propanoate,
Methyl α-hydroxy-β-methylbicyclo[2.2.1]heptane-2-propanoate,
α,β-Dihydroxy-β-methylbicyclo[2.2.1]heptane-2-propanoic acid,
Butyl 3-bicyclo[2.2.1]hept-5-en-2-yl-2-propenoate,
Tert-Butyl 3-bicyclo[2.2.1]hept-5-en-2-yl-2-propenoate,
β-Cyclobutylbicyclo[2.2.1]heptane-2-propanoic acid,
α-Cyclopropyl-α-methylbicyclo[2.2.1]heptane-2-propanoic acid,
Ethyl β,β-dimethylbicyclo[2.2.1]hept-5-ene-2-propanoate,
Tert-butyl bicyclo[2.2.1]hept-5-ene-2-propanoate,
Butyl 3-bicyclo[2.2.1]hept-2-yl-2-propenoate,
Ethyl 3-bicyclo[2.2.1]hept-2-yl-2-methyl-2-butenoate,
Ethyl 2-(bicyclo[2.2.1]hept-2-ylmethylene)butanoate,
Tert-butyl 3-bicyclo[2.2.1]hept-2-yl-2-propenoate,
Methyl α-hydroxy-β,β-dimethylbicyclo[2.2.1]hept-5-ene-2-propanoate,
Methyl α-ethyl-β-oxobicyclo[2.2.1]heptane-2-propanoate,
α,α-Diethylbicyclo[2.2.1]heptane-2-propanoic acid,
α-Isobutylbicyclo[2.2.1]heptane-2-propanoic acid,
α-tert-Butylbicyclo[2.2.1]heptane-2-propanoic acid,
α-Isopropyl-α-methylbicyclo[2.2.1]heptane-2-propanoic acid,
α-Methyl-α-propylbicyclo[2.2.1]heptane-2-propanoic acid,
Butyl bicyclo[2.2.1]heptane-2-propanoate,
Tert-butyl bicyclo[2.2.1]heptane-2-propanoate,
Ethyl α-hydroxy-β-methylbicyclo[2.2.1]heptane-2-propanoate,
Methyl α-hydroxy-β,β-dimethylbicyclo[2.2.1]heptane-2-propanoate,
Pentyl 3-bicyclo[2.2.1]hept-5-en-2-yl-2-propenoate,
Tert-Butyl β-oxobicyclo[2.2.1]hept-5-ene-2-propanoate,
α-Cyclopentylbicyclo[2.2.1]heptane-2-propanoic acid,
Tert-Butyl β-methylbicyclo[2.2.1]hept-5-ene-2-propanoate,
Tert-Butyl 3-bicyclo[2.2.1]hept-2-yl-2-butenoate,
Ethyl α-ethenyl-α-methylbicyclo[2.2.1]heptane-2-propanoate,
Tert-Butyl β-hydroxybicyclo[2.2.1]hept-5-ene-2-propanoate,
Tert-Butyl α-hydroxybicyclo[2.2.1]hept-5-ene-2-propanoate,
Ethyl α-ethyl-β-oxobicyclo[2.2.1]heptane-2-propanoate,
Methyl α-isopropyl-β-oxobicyclo[2.2.1]heptane-2-propanoate,
Ethyl α,α-dimethyl-β-oxobicyclo[2.2.1]heptane-2-propanoate,
Butyl α-methylbicyclo[2.2.1]heptane-2-propanoate,
Tert-Butyl β-methylbicyclo[2.2.1]heptane-2-propanoate,
α-Hydroxy-β-methyl-α-(1-methylethyl)bicyclo[2.2.1]heptane-2-propanoic acid,
β-Hydroxy-α-isopropylbicyclo[2.2.1]heptane-2-propanoic acid,
α-Hydroxy-β-methyl-α-propylbicyclo[2.2.1]heptane-2-propanoic acid,
Tert-Butyl β-hydroxybicyclo[2.2.1]heptane-2-propanoate,
Ethyl α-hydroxy-α,β-dimethylbicyclo[2.2.1]heptane-2-propanoate,
Tert-Butyl β-hydroxy-β-methylbicyclo[2.2.1]hept-5-ene-2-propanoate,
Ethyl β-oxo-α-propylbicyclo[2.2.1]heptane-2-propanoate,
Methyl α-butyl-β-oxobicyclo[2.2.1]heptane-2-propanoate,
Ethyl α-isopropyl-β-oxobicyclo[2.2.1]heptane-2-propanoate,
Tert-Butyl α-methyl-β-oxobicyclo[2.2.1]heptane-2-propanoate,
α-Butyl-α-ethylbicyclo[2.2.1]heptane-2-propanoic acid,
Methyl 2-methyl-β-oxobicyclo[2.2.1]hept-5-ene-2-propanoate,
4-Bicyclo[2.2.1]hept-5-en-2-yl-3-butenoic acid,
Bicyclo[2.2.1]hept-5-ene-2-butanoic acid,
4-Bicyclo[2.2.1]hept-2-yl-3-butenoic acid,
4-Bicyclo[2.2.1]hept-2-yl-2-butenoic acid,
Bicyclo[2.2.1]heptane-2-butanoic acid,
γ-Oxobicyclo[2.2.1]hept-5-ene-2-butanoic acid,
α-Methylbicyclo[2.2.1]hept-5-ene-2-butanoic acid,
Methyl bicyclo[2.2.1]hept-5-ene-2-butanoate,
α-Methylbicyclo[2.2.1]heptane-2-butanoic acid,
β-Methylbicyclo[2.2.1]heptane-2-butanoic acid,
α-Hydroxybicyclo[2.2.1]heptane-2-butanoic acid,
Methyl γ-oxobicyclo[2.2.1]hept-5-ene-2-butanoate,
a-Methyl-γ-oxobicyclo[2.2.1]heptane-2-butanoic acid,
β-Methyl-γ-oxobicyclo[2.2.1]heptane-2-butanoic acid,
Methyl γ-oxobicyclo[2.2.1]heptane-2-butanoate,
Methyl b-oxobicyclo[2.2.1]heptane-2-butanoate,
α,β-Dimethylbicyclo[2.2.1]heptane-2-butanoic acid,
β,β-Dimethylbicyclo[2.2.1]heptane-2-butanoic acid,
α-Hydroxy-β-oxobicyclo[2.2.1]heptane-2-butanoic acid,
β-Hydroxy-α-methylbicyclo[2.2.1]heptane-2-butanoic acid,
γ-Hydroxy-α-methylbicyclo[2.2.1]heptane-2-butanoic acid,
γ-Hydroxy-β-methylbicyclo[2.2.1]heptane-2-butanoic acid,
Methyl γ-hydroxybicyclo[2.2.1]heptane-2-butanoate,
Ethyl γ-oxobicyclo[2.2.1]hept-5-ene-2-butanoate,
Methyl α,γ-dioxobicyclo[2.2.1]heptane-2-butanoate,
α,β-Dimethyl-γ-oxobicyclo[2.2.1]heptane-2-butanoic acid,
Ethyl γ-oxobicyclo[2.2.1]heptane-2-butanoate,
Methyl α-methyl-γ-oxobicyclo[2.2.1]heptane-2-butanoate,
Methyl β-methyl-γ-oxobicyclo[2.2.1]heptane-2-butanoate,
Ethyl β-oxobicyclo[2.2.1]heptane-2-butanoate,
Methyl α-methyl-β-oxobicyclo[2.2.1]heptane-2-butanoate,
γ-Hydroxy-α,β-dimethylbicyclo[2.2.1]heptane-2-butanoic acid,
α-Hydroxy-γ,γ-dimethylbicyclo[2.2.1]heptane-2-butanoic acid,
Methyl γ-hydroxy-β-methylbicyclo[2.2.1]heptane-2-butanoate,
Methyl γ-hydroxy-α-methylbicyclo[2.2.1]heptane-2-butanoate,
Propyl γ-oxobicyclo[2.2.1]heptane-2-butanoate,
Methyl α-ethyl-β-oxobicyclo[2.2.1]heptane-2-butanoate,
Propyl γ-hydroxybicyclo[2.2.1]heptane-2-butanoate,
Tert-butyl β-hydroxybicyclo[2.2.1]hept-5-ene-2-butanoate,
Tert-butyl α-methyl-bicyclo[2.2.1]heptane-2-butanoate,
Bicyclo[2.2.1]hept-5-ene-2-pentanoic acid,
Bicyclo[2.2.1]heptane-2-pentanoic acid,
5-Bicyclo[2.2.1]hept-2-yl-3-methyl-2,4-pentadienoic acid,
δ-Oxobicyclo[2.2.1]hept-5-ene-2-pentanoic acid,
Methyl bicyclo[2.2.1]hept-5-ene-2-pentanoate,
δ-Oxobicyclo[2.2.1]heptane-2-pentanoic acid,
γ-Oxobicyclo[2.2.1]heptane-2-pentanoic acid,
β-Methylbicyclo[2.2.1]heptane-2-pentanoic acid,
δ-Methylbicyclo[2.2.1]heptane-2-pentanoic acid,
Methyl 5-bicyclo[2.2.1]hept-5-en-2-yl-3-oxo-4-pentenoate,
5-Bicyclo[2.2.1]hept-2-yl-3-methyl-2,4-hexadienoic acid,
Methyl 5-bicyclo[2.2.1]hept-2-yl-3-methyl-2,4-pentadienoate,
Methyl δ-oxobicyclo[2.2.1]hept-5-ene-2-pentanoate,
Methyl γ-oxobicyclo[2.2.1]heptane-2-pentanoate,
β,δ-Dimethylbicyclo[2.2.1]heptane-2-pentanoic acid,
β-Hydroxy-α-methylbicyclo[2.2.1]heptane-2-pentanoic acid,
Methyl 5-bicyclo[2.2.1]hept-2-yl-3-methyl-2,4-hexadienoate,
Ethyl β-oxobicyclo[2.2.1]hept-5-ene-2-pentanoate,
Methyl 5-bicyclo[2.2.1]hept-2-yl-3-methyl-5-oxo-2-pentenoate,
Ethyl γ-oxobicyclo[2.2.1]heptane-2-pentanoate,
1-Bicyclo[2.2.1]hept-5-en-2-yl butanedioate,
1-bicyclo[2.2.1]hept-2-yl butanedioate,
1-Bicyclo[2.2.1]hept-2-yl 2-hydroxybutanedioate,
1-Bicyclo[2.2.1]hept-2-yl 4-ethyl butanedioate,
Ethyl 4-[(1S,2R,4R)-bicyclo[2.2.1]hept-2-yloxy]-3-oxobutanoate,
4-Bicyclo[2.2.1]hept-2-yl 2,3-dihydroxy-butanedioate,
1-Bicyclo[2.2.1]hept-2-yl 4-propyl butanedioate,
1-Bicyclo[2.2.1]hept-2-yl 4-isopropyl butanedioate,
1-Bicyclo[2.2.1]hept-2-yl 4-butyl butanedioate,
1-Bicyclo[2.2.1]hept-2-yl 4-pentyl butanedioate,
1-Bicyclo[2.2.1]hept-2-yl 4-hexyl butanedioate,
1-Bicyclo[2.2.1]hept-2-yl 4-heptyl butanedioate,
1-Bicyclo[2.2.1]hept-2-yl propanedioate,
1-Bicyclo[2.2.1]hept-2-yl 3-ethyl propanedioate,
1-Bicyclo[2.2.1]hept-2-yl 3-propyl propanedioate,
1-Bicyclo[2.2.1]hept-2-yl 3-isopropyl propanedioate,
1-Bicyclo[2.2.1]hept-2-yl 3-butyl propanedioate,
1-Bicyclo[2.2.1]hept-2-yl 3-pentyl propanedioate,
1-Bicyclo[2.2.1]hept-2-yl 3-hexyl propanedioate,
1-Bicyclo[2.2.1]hept-2-yl 3-heptyl propanedioate,
2-(Bicyclo[2.2.1]hept-5-en-2-yloxy)acetic acid,
2-(Bicyclo[2.2.1]hept-2-yloxy)acetic acid,
Methyl 2-(bicyclo[2.2.1]hept-5-en-2-yloxy)acetate,
1-Bicyclo[2.2.1]hept-2-yl ethanedioate,
2-(Bicyclo[2.2.1]hept-2-yloxy)propanoic acid,
Ethyl (bicyclo[2.2.1]hept-5-en-2-yloxy)acetate,
Ethyl 2-(bicyclo[2.2.1]hept-2-yloxy)acetate,
2-(Bicyclo[2.2.1]hept-2-yloxy)-3-methylbutanoic acid,
Ethyl 2-(bicyclo[2.2.1]hept-2-yloxy)propanoate,
1-Bicyclo[2.2.1]hept-2-yl 2-(2-propen-1-yl) ethanedioate,
2-(Bicyclo[2.2.1]hept-5-en-2-ylmethoxy)acetic acid,
2-(Bicyclo[2.2.1]hept-2-ylmethoxy)acetic acid,
Methyl 2-(bicyclo[2.2.1]hept-5-en-2-ylmethoxy)acetate,
2-(Bicyclo[2.2.1]hept-2-ylmethoxy)propanoic acid,
2-(1-Bicyclo[2.2.1]hept-5-en-2-yl-1-methylethoxy)acetic acid,
1-Carboxyethyl Bicyclo[2.2.1]hept-5-ene-2-carboxylate,
2-Methoxy-1-methyl-2-oxoethyl bicyclo[2.2.1]hept-5-ene-2-carboxylate,
1-(Bicyclo[2.2.1]hept-5-en-2-ylmethyl) 2-ethyl ethanedioate,
2-Ethoxy-2-oxoethyl bicyclo[2.2.1]heptane-2-carboxylate,
2-Ethoxy-1-methyl-2-oxoethyl bicyclo[2.2.1]hept-5-ene-2-carboxylate,
1-(1-bicyclo[2.2.1]hept-5-en-2-ylethyl) 2-ethyl ethanedioate,
2-t-butoxy-2-oxoethyl bicyclo[2.2.1]heptane-2-carboxylate,
2-Isopropoxy-1-methyl-2-oxoethyl bicyclo[2.2.1]hept-5-ene-2-carboxylate,
1-(1-bicyclo[2.2.1]hept-5-en-2-ylpropyl) 2-ethyl ethanedioate,
1-Cyclopropyl-2-methoxy-2-oxoethyl bicyclo[2.2.1]heptane-2-carboxylate,
2-(Cyclohexyloxy)-1-methyl-2-oxoethyl bicyclo[2.2.1]hept-5-ene-2-carboxylate,
2-Carboxyethyl bicyclo[2.2.1]hept-5-ene-2-carboxylate,
2-Carboxyethyl bicyclo[2.2.1]heptane-2-carboxylate,
1-(1-Bicyclo[2.2.1]hept-5-en-2-ylethyl) 3-ethyl propanedioate,
1-(1-Bicyclo[2.2.1]hept-5-en-2-ylpropyl) 3-ethyl propanedioate,
3-(t-butoxy)-3-oxopropyl bicyclo[2.2.1]hept-5-ene-2-carboxylate,
3-(t-butoxy)-1-hydroxy-3-oxopropyl bicyclo[2.2.1]hept-5-ene-2-carboxylate,
6-Bicyclo[2.2.1]hept-5-en-2-yl-5-hepten-2-one,
1-Bicyclo[2.2.1]hept-5-en-2-yl-1,5-hexanedione,
5-Bicyclo[2.2.1]hept-2-yl-3-methyl-2,4-pentadienal,
5-Bicyclo[2.2.1]hept-2-yl-4-methyl-4-pentenal,
γ-Methylbicyclo[2.2.1]heptane-2-pentanal,
5-Bicyclo[2.2.1]hept-2-yl-2,4-dimethyl-4-pentenal,
Bicyclo[2.2.1]hept-5-ene-2-butanal,
4-Bicyclo[2.2.1]hept-2-yl-3-butenal,
Bicyclo[2.2.1]heptane-2-butanal,
β-Oxobicyclo[2.2.1]hept-5-ene-2-butanal,
4-Bicyclo[2.2.1]hept-5-en-2-yl-4-hydroxy-2-butenal,
α-Methylbicyclo[2.2.1]heptane-2-butanal,
γ-Hydroxybicyclo[2.2.1]heptane-2-butanal,
α-Hydroxy-β-oxobicyclo[2.2.1]heptane-2-butanal,
γ-Hydroxy-T-methylbicyclo[2.2.1]heptane-2-butanal,
β-Hydroxy-β-methylbicyclo[2.2.1]heptane-2-butanal,
β-Hydroxy-β-methyl-γ-oxobicyclo[2.2.1]heptane-2-butanal,
α-Hydroxy-α-methyl-β-oxobicyclo[2.2.1]heptane-2-butanal,
5-Bicyclo[2.2.1]hept-5-en-2-yl-2-pentanone,
5-Bicyclo[2.2.1]hept-2-yl-2-pentanone,
1-Bicyclo[2.2.1]hept-5-en-2-yl-1,4-pentanedione,
1-Bicyclo[2.2.1]hept-2-yl-1,4-pentanedione,
6-Bicyclo[2.2.1]hept-2-yl-3-hexanone,
5-Bicyclo[2.2.1]hept-2-yl-4-methyl-2-pentanone,
1-Bicyclo[2.2.1]hept-5-en-2-yl-1,4-hexanedione,
4-Bicyclo[2.2.1]hept-2-yl-1-cyclopropyl-1-butanone,
1-Bicyclo[2.2.1]hept-2-yl-2-methyl-1,4-pentanedione,
1-Bicyclo[2.2.1]hept-2-yl-4-heptanone,
6-Bicyclo[2.2.1]hept-2-yl-2-methyl-3-hexanone,
5-Bicyclo[2.2.1]hept-2-yl-4,4-dimethyl-2-pentanone,
1-Bicyclo[2.2.1]hept-5-en-2-yl-1,4-heptanedione,
1-Bicyclo[2.2.1]hept-5-en-2-yl-5-methyl-1,4-hexanedione,
6-Bicyclo[2.2.1]hept-2-yl-2,2-dimethyl-3-hexanone,
1-Bicyclo[2.2.1]hept-5-en-2-yl-1,4-octanedione,
4-Bicyclo[2.2.1]hept-2-yl-1-phenyl-1-butanone,
1-Bicyclo[2.2.1]hept-5-en-2-yl-1,4-decanedione,
3-Bicyclo[2.2.1]hept-5-en-2-yl-2-propenal,
Bicyclo[2.2.1]hept-5-ene-2-propanal,
3-Bicyclo[2.2.1]hept-2-yl-2-propenal,
Bicyclo[2.2.1]heptane-2-propanal,
3-Bicyclo[2.2.1]hept-5-en-2-yl-2-methyl-2-propenal,
β-Oxobicyclo[2.2.1]hept-5-ene-2-propanal,
3-Bicyclo[2.2.1]hept-2-yl-2-methyl-2-propenal,
β-Oxobicyclo[2.2.1]heptane-2-propanal,
α-Methylbicyclo[2.2.1]heptane-2-propanal,
β-Methylbicyclo[2.2.1]heptane-2-propanal,
α-Hydroxybicyclo[2.2.1]heptane-2-propanal,
β-Hydroxybicyclo[2.2.1]heptane-2-propanal,
2-(Bicyclo[2.2.1]hept-5-en-2-ylmethylene)butanal,
2-(Bicyclo[2.2.1]hept-2-ylmethylene)butanal,
α,α-Dimethylbicyclo[2.2.1]heptane-2-propanal,
α-Hydroxy-β-oxobicyclo[2.2.1]heptane-2-propanal,
β-Hydroxy-β-methylbicyclo[2.2.1]heptane-2-propanal,
α-Hydroxy-α-methylbicyclo[2.2.1]heptane-2-propanal,
2-(Bicyclo[2.2.1]hept-2-ylmethylene)-3-methylbutanal,
α-Ethyl-α-methylbicyclo[2.2.1]heptane-2-propanal,
α-Hydroxy-α-methyl-β-oxobicyclo[2.2.1]heptane-2-propanal,
2-(Bicyclo[2.2.1]hept-5-en-2-ylmethylene)hexanal,
2-(Bicyclo[2.2.1]hept-5-en-2-ylmethylene)heptanal,
2-Methyl-β-oxobicyclo[2.2.1]hept-5-ene-2-propanal,
2-Butylbicyclo[2.2.1]heptane-2-propanal,
4-(2-Methylbicyclo[2.2.1]hept-5-en-2-yl)-3-buten-2-one,
1-(2-Methylbicyclo[2.2.1]hept-5-en-2-yl)-1-penten-3-one,
1-(2-Methylbicyclo[2.2.1]hept-5-en-2-yl)-1,3-butanedione,
4-Bicyclo[2.2.1]hept-5-en-2-yl-3-buten-2-one,
4-Bicyclo[2.2.1]hept-5-en-2-yl-2-butanone,
4-Bicyclo[2.2.1]hept-2-yl-3-buten-2-one,
4-Bicyclo[2.2.1]hept-2-yl-2-butanone,
4-Bicyclo[2.2.1]hept-5-en-2-yl-3-penten-2-one,
1-Bicyclo[2.2.1]hept-5-en-2-yl-1-penten-3-one,
4-Bicyclo[2.2.1]hept-5-en-2-yl-3-methyl-3-buten-2-one,
1-Bicyclo[2.2.1]hept-5-en-2-yl-1,3-butanedione,
4-Bicyclo[2.2.1]hept-2-yl-3-penten-2-one,
4-Bicyclo[2.2.1]hept-2-yl-3-methyl-3-buten-2-one,
4-Bicyclo[2.2.1]hept-5-en-2-yl-4-hydroxy-2-butanone,
1-Bicyclo[2.2.1]hept-2-yl-1,3-butanedione,
1-Bicyclo[2.2.1]hept-2-yl-2,3-butanedione,
1-Bicyclo[2.2.1]hept-2-yl-3-pentanone,
4-Bicyclo[2.2.1]hept-2-yl-3-methyl-2-butanone,
4-Bicyclo[2.2.1]hept-2-yl-2-pentanone,
4-Bicyclo[2.2.1]hept-2-yl-3-hydroxy-2-butanone,
4-Bicyclo[2.2.1]hept-2-yl-4-hydroxy-2-butanone,
1-Bicyclo[2.2.1]hept-2-yl-3-hydroxy-2-butanone,
1-Bicyclo[2.2.1]hept-5-en-2-yl-4-pentene-1,3-dione,
1-Bicyclo[2.2.1]hept-5-en-2-yl-1-hexen-3-one,
3-(Bicyclo[2.2.1]hept-5-en-2-ylmethylene)-2-pentanone,
1-Bicyclo[2.2.1]hept-5-en-2-yl-2-methyl-1-penten-3-one,
1-Bicyclo[2.2.1]hept-5-en-2-yl-4-methyl-1-penten-3-one,
1-Bicyclo[2.2.1]hept-5-en-2-yl-1,3-pentanedione,
1-Bicyclo[2.2.1]hept-2-yl-2-methyl-1-penten-3-one,
3-Bicyclo[2.2.1]hept-2-yl-1-cyclopropyl-1-propanone,
1-Bicyclo[2.2.1]hept-2-yl-2-methyl-1,3-butanedione,
1-Bicyclo[2.2.1]hept-2-yl-1,3-pentanedione,
1-Bicyclo[2.2.1]hept-2-yl-3-hexanone,
3-(Bicyclo[2.2.1]hept-2-ylmethyl)-2-pentanone,
1-Bicyclo[2.2.1]hept-2-yl-2-methyl-3-pentanone,
1-Bicyclo[2.2.1]hept-2-yl-4-methyl-3-pentanone,
4-Bicyclo[2.2.1]hept-2-yl-4-methyl-2-pentanone,
1-Bicyclo[2.2.1]hept-2-yl-1-hydroxy-2,3-butanedione,
1-Bicyclo[2.2.1]hept-2-yl-3-hydroxy-2-pentanone,
4-Bicyclo[2.2.1]hept-2-yl-4-hydroxy-2-pentanone,
1-Bicyclo[2.2.1]hept-5-en-2-yl-5-methyl-1,4-hexadien-3-one,
1-Bicyclo[2.2.1]hept-2-yl-1,6-heptadien-3-one,
1-Bicyclo[2.2.1]hept-2-yl-3-cyclopropyl-1,3-propanedione,
1-Bicyclo[2.2.1]hept-2-yl-6-hepten-3-one,
1-Bicyclo[2.2.1]hept-2-yl-1,3-hexanedione,
1-Bicyclo[2.2.1]hept-2-yl-2-methyl-1,3-pentanedione,
1-Bicyclo[2.2.1]hept-2-yl-4-methyl-1,3-pentanedione,
3-(Bicyclo[2.2.1]hept-2-ylmethyl)-4-methyl-2-pentanone,
1-Bicyclo[2.2.1]hept-2-yl-5-methyl-3-hexanone,
1-Bicyclo[2.2.1]hept-2-yl-4,4-dimethyl-3-pentanone,
4-Bicyclo[2.2.1]hept-2-yl-4-hydroxy-2,3-pentanedione,
1-Bicyclo[2.2.1]hept-2-yl-3-hydroxy-2-hexanone,
1-Bicyclo[2.2.1]hept-2-yl-3-hydroxy-4-methyl-2-pentanone,
3-Bicyclo[2.2.1]hept-2-yl-1-cyclopentyl-1-propanone,
1-Bicyclo[2.2.1]hept-2-yl-4-methyl-1,3-hexanedione,
1-Bicyclo[2.2.1]hept-2-yl-5-methyl-1,3-hexanedione,
1-Bicyclo[2.2.1]hept-2-yl-2,4-dimethyl-1,3-pentanedione,
1-Bicyclo[2.2.1]hept-2-yl-4,4-dimethyl-1,3-pentanedione,
3-Bicyclo[2.2.1]hept-5-en-2-yl-1-phenyl-2-propen-i-one,
3-Bicyclo[2.2.1]hept-2-yl-1-phenyl-1-propanone,
1-Bicyclo[2.2.1]hept-2-yl-3-cyclopentyl-1,3-propanedione,
1-Bicyclo[2.2.1]hept-2-yl-1,3-octanedione,
1-Bicyclo[2.2.1]hept-2-yl-4-ethyl-1,3-hexanedione,
1-Bicyclo[2.2.1]hept-2-yl-2,4-dimethyl-1,3-hexanedione,
1-Bicyclo[2.2.1]hept-2-yl-2,5-dimethyl-1,3-hexanedione,
1-Bicyclo[2.2.1]hept-2-yl-5,5-dimethyl-1,3-hexanedione,
1-Bicyclo[2.2.1]hept-2-yl-2,4,4-trimethyl-1,3-pentanedione,
1-Bicyclo[2.2.1]hept-2-yl-4-ethyl-3-hydroxy-2-hexanone,
1-Bicyclo[2.2.1]hept-2-yl-3-cyclopentyl-2-methyl-1,3-propanedione,
1-Bicyclo[2.2.1]hept-2-yl-4-ethyl-2-methyl-1,3-hexanedione,
1-Bicyclo[2.2.1]hept-2-yl-2,5,5-trimethyl-1,3-hexanedione,
2-(Bicyclo[2.2.1]hept-2-yloxy)acetaldehyde,
2-Methylbicyclo[2.2.1]hept-5-en-2-yl 3-oxobutanoate,
Bicyclo[2.2.1]hept-5-en-2-yl 3-oxobutanoate,
2-Oxopropyl bicyclo[2.2.1]hept-5-ene-2-carboxylate,
1-(1-Bicyclo[2.2.1]hept-5-en-2-ylethoxy)-2-propanone,
2-Oxo-2-phenylethyl bicyclo[2.2.1]hept-5-ene-2-carboxylate,
1-Methyl-2-oxo-2-phenylethyl bicyclo[2.2.1]heptane-2-carboxylate,
Bicyclo[2.2.1]hept-2-yl 4-oxopentanoate,
Bicyclo[2.2.1]hept-2-yl 7-oxobenzenebutanoate,
Bicyclo[2.2.1]hept-5-en-2-ylmethyl 3-oxobutanoate,
1,1-Dimethyl-3-oxobutyl bicyclo[2.2.1]hept-5-ene-2-carboxylate,
1-(1-Methylethyl)-3-oxobutyl bicyclo[2.2.1]hept-5-ene-2-carboxylate,
3-(Bicyclo[2.2.1]hept-5-en-2-ylmethoxy)-2-oxobutanal,
1,1-Dimethyl-3-oxobutyl 2-methylbicyclo[2.2.1]hept-5-ene-2-carboxylate,
2-Oxo-2-phenylethyl bicyclo[2.2.1]heptane-2-acetate,
1-Methyl-2-oxo-2-phenylethyl bicyclo[2.2.1]heptane-2-acetate,
α,2-Dimethyl-bicyclo[2.2.1]hept-5-ene-2-propanol, 2-acetate,
α,2-Dimethyl-bicyclo[2.2.1]heptane-2-propanol, 2-acetate,
Bicyclo[2.2.1]hept-5-ene-2-propanol, 2-acetate,
Bicyclo[2.2.1]heptane-2-propanol, 2-acetate,
4-Bicyclo[2.2.1]hept-5-en-2-yl-3-buten-2-ol, 2-acetate,
α-Methyl-bicyclo[2.2.1]heptane-2-propanol, 2-acetate,
2-(Bicyclo[2.2.1]hept-5-en-2-ylmethylene)-1-butanol, 1-acetate,
b-Ethyl-bicyclo[2.2.1]heptane-2-propanol, 2-acetate,
3-Bicyclo[2.2.1]hept-5-en-2-yl-2-methyl-2-propen-1-ol, 1-acetate,
3-Bicyclo[2.2.1]hept-2-yl-1,1-dimethylpropyl 2-methyl-2-propenoate,
2-Bicyclo[2.2.1]hept-5-en-2-yl-ethenol, 1-acetate,
Bicyclo[2.2.1]hept-5-ene-2-ethanol, 2-acetate,
1-Bicyclo[2.2.1]hept-5-en-2-yl-1-propen-2-ol, acetate,
2-(Acetyloxy)-1-bicyclo[2.2.1]hept-5-en-2-ylethanone,
Bicyclo[2.2.1]heptane-2-ethanol, 2-propanoate,
α,β-Dimethyl-bicyclo[2.2.1]heptane-2-ethanol, 2-acetate,
Bicyclo[2.2.1]hept-5-ene-2-methanol, 2-acetate,
Bicyclo[2.2.1]heptane-2-methanol, 2-acetate,
Bicyclo[2.2.1]hept-5-en-2-ylmethyl 2-propenoate,
α-Methyl-bicyclo[2.2.1]hept-5-ene-2-methanol, 2-acetate,
Bicyclo[2.2.1]hept-2-ylmethyl 2-propenoate,
α-Methyl-bicyclo[2.2.1]heptane-2-methanol, 2-acetate,
Bicyclo[2.2.1]heptane-2-methanol, 2-propanoate,
Bicyclo[2.2.1]hept-5-en-2-ylmethyl 2-methyl-2-propenoate,
Bicyclo[2.2.1]hept-5-en-2-ylmethyl 2-butenoate,
Bicyclo[2.2.1]hept-5-en-2-ylmethyl butanoate,
α-Methyl-bicyclo[2.2.1]hept-5-ene-2-methanol, propanoate,
Bicyclo[2.2.1]hept-5-en-2-ylmethyl 2-methylpropanoate,
α-Ethyl-bicyclo[2.2.1]hept-5-ene-2-methanol, propanoate,
α,α-Dimethyl-bicyclo[2.2.1]hept-5-ene-2-methanol, acetate
Bicyclo[2.2.1]hept-2-ylmethyl cyclopropanecarboxylate,
Bicyclo[2.2.1]hept-2-ylmethyl 2-methyl-2-propenoate,
Bicyclo[2.2.1]hept-2-ylmethyl butanoate,
Bicyclo[2.2.1]hept-2-ylmethyl 2-methylpropanoate,
α-Ethyl-bicyclo[2.2.1]heptane-2-methanol, 2-acetate,
α,α-Dimethyl-bicyclo[2.2.1]heptane-2-methanol, acetate,
Bicyclo[2.2.1]hept-5-en-2-ylmethyl pentanoate,
1-Bicyclo[2.2.1]hept-5-en-2-ylethyl butanoate,
Bicyclo[2.2.1]hept-5-en-2-ylmethyl 3-methylbutanoate,
α-Propyl-bicyclo[2.2.1]hept-5-ene-2-methanol, 2-acetate,
α-Isopropyl-bicyclo[2.2.1]hept-5-ene-2-methanol, 2-acetate,
Bicyclo[2.2.1]hept-5-en-2-ylmethyl 2,2-dimethylpropanoate,
1-Bicyclo[2.2.1]hept-2-yl-1-methylethyl 2-propenoate,
1-Bicyclo[2.2.1]hept-2-ylethyl butanoate,
Bicyclo[2.2.1]hept-2-ylmethyl pentanoate,
Bicyclo[2.2.1]hept-2-ylmethyl 3-methylbutanoate,
Bicyclo[2.2.1]hept-5-en-2-ylmethyl hexanoate,
1-Bicyclo[2.2.1]hept-5-en-2-ylpropyl butanoate,
α-Butyl-bicyclo[2.2.1]hept-5-ene-2-methanol, 2-acetate,
α-Isobutyl-bicyclo[2.2.1]hept-5-ene-2-methanol, 2-acetate,
1-Bicyclo[2.2.1]hept-2-yl-1-methylethyl 2-methyl-2-propenoate,
α-Butyl-bicyclo[2.2.1]heptane-2-methanol, 2-acetate,
1-Bicyclo[2.2.1]hept-5-ene-2-yl-1-methylethyl 2-methyl-2-propenoate,
2-[(2-Methylbicyclo[2.2.1]hept-2-yl)oxy]-2-oxoethyl 2-methyl-2-propenoate,
2-[(2-Ethylbicyclo[2.2.1]hept-2-yl)oxy]-2-oxoethyl 2-methyl-2-propenoate,
2-Bicyclo[2.2.1]hept-2-yloxyethanol, 1-acetate,
Bicyclo[2.2.1]hept-5-en-2-yl 2-(acetyloxy)acetate,
2-(Bicyclo[2.2.1]hept-2-yloxy)ethyl 2-propenoate,
2-Bicyclo[2.2.1]hept-2-yloxyethanol, 1-propanoate,
2-(Bicyclo[2.2.1]hept-2-yloxy)ethyl 2-methyl-2-propenoate,
2-Bicyclo[2.2.1]hept-2-yloxyethyl 2-methylpropanoate,
2-Bicyclo[2.2.1]hept-5-en-2-yloxyethanol, 1-benzoate,
Bicyclo[2.2.1]hept-5-en-2-ylmethyl 3-pyridinecarboxylate,
Bicyclo[2.2.1]hept-5-en-2-ylmethyl 2-thiophenecarboxylate,
Bicyclo[2.2.1]hept-5-ene-2-methanol, 2-benzoate,
Bicyclo[2.2.1]heptane-2-methanol, 2-benzoate,
2-Methylbicyclo[2.2.1]hept-5-ene-2-carboxylic acid,
2-Methylbicyclo[2.2.1]heptane-2-carboxylic acid,
2-Ethylbicyclo[2.2.1]hept-5-ene-2-carboxylic acid,
Methyl 2-methylbicyclo[2.2.1]hept-5-ene-2-carboxylate,
2-Ethylbicyclo[2.2.1]heptane-2-carboxylic acid,
Methyl 2-methylbicyclo[2.2.1]heptane-2-carboxylate,
2-Propylbicyclo[2.2.1]hept-5-ene-2-carboxylic acid,
Methyl 2-ethylbicyclo[2.2.1]hept-5-ene-2-carboxylate,
Ethyl 2-methylbicyclo[2.2.1]hept-5-ene-2-carboxylate,
Ethenyl 2-methylbicyclo[2.2.1]heptane-2-carboxylate,
2-Propylbicyclo[2.2.1]heptane-2-carboxylic acid,
Methyl 2-ethylbicyclo[2.2.1]heptane-2-carboxylate,
Ethyl 2-methylbicyclo[2.2.1]heptane-2-carboxylate,
2-Propen-1-yl 2-methylbicyclo[2.2.1]hept-5-ene-2-carboxylate,
2-(2-Methylpropyl)bicyclo[2.2.1]hept-5-ene-2-carboxylic acid,
2-Butylbicyclo[2.2.1]hept-5-ene-2-carboxylic acid,
2-Isobutylbicyclo[2.2.1]hept-5-ene-2-carboxylic acid
Methyl 2-propylbicyclo[2.2.1]hept-5-ene-2-carboxylate,
Ethyl 2-ethylbicyclo[2.2.1]hept-5-ene-2-carboxylate,
2-(3-Buten-1-yl)bicyclo[2.2.1]heptane-2-carboxylic acid,
Propyl 2-methylbicyclo[2.2.1]hept-5-ene-2-carboxylate,
1-Isopropyl 2-methylbicyclo[2.2.1]hept-5-ene-2-carboxylate,
2-(2-Buten-1-yl)bicyclo[2.2.1]heptane-2-carboxylic acid,
2-(2-Methyl-2-propen-1-yl)bicyclo[2.2.1]heptane-2-carboxylic acid,
2-Butylbicyclo[2.2.1]heptane-2-carboxylic acid,
2-Isobutylbicyclo[2.2.1]heptane-2-carboxylic acid,
Ethyl 2-ethylbicyclo[2.2.1]heptane-2-carboxylate,
2-Pentylbicyclo[2.2.1]hept-5-ene-2-carboxylic acid,
Methyl 2-butylbicyclo[2.2.1]hept-5-ene-2-carboxylate,
Methyl 2-isobutylbicyclo[2.2.1]heptane-2-carboxylate,
2-(4-Penten-1-yl)bicyclo[2.2.1]heptane-2-carboxylic acid,
Isobutyl 2-methylbicyclo[2.2.1]hept-5-ene-2-carboxylate,
2-(3-Methyl-3-buten-1-yl)bicyclo[2.2.1]heptane-2-carboxylic acid,
Tert-Butyl 2-methylbicyclo[2.2.1]hept-5-ene-2-carboxylate,
2-(3-Methyl-2-buten-1-yl)bicyclo[2.2.1]heptane-2-carboxylic acid,
2-(2-Methylenebutyl)bicyclo[2.2.1]heptane-2-carboxylic acid,
2-Pentylbicyclo[2.2.1]heptane-2-carboxylic acid,
2-(2,2-Dimethylpropyl)bicyclo[2.2.1]heptane-2-carboxylic acid,
2-(2-Methylbutyl)bicyclo[2.2.1]heptane-2-carboxylic acid,
2-(3-Methylbutyl)bicyclo[2.2.1]heptane-2-carboxylic acid,
Propyl 2-ethylbicyclo[2.2.1]heptane-2-carboxylate,
Isopropyl 2-ethylbicyclo[2.2.1]heptane-2-carboxylate,
Pentyl 2-methylbicyclo[2.2.1]hept-5-ene-2-carboxylate,
Ethyl 2-butylbicyclo[2.2.1]hept-5-ene-2-carboxylate,
Bicyclo[2.2.1]hept-5-ene-2-carboxylic acid,
Bicyclo[2.2.1]heptane-2-carboxylic acid,
Methyl bicyclo[2.2.1]hept-5-ene-2-carboxylate,
Methyl bicyclo[2.2.1]heptane-2-carboxylate,
Ethyl bicyclo[2.2.1]hept-5-ene-2-carboxylate,
Ethenyl bicyclo[2.2.1]heptane-2-carboxylate
Ethyl bicyclo[2.2.1]heptane-2-carboxylate,
Isopropyl bicyclo[2.2.1]hept-5-ene-2-carboxylate,
Propyl bicyclo[2.2.1]hept-5-ene-2-carboxylate,
2-Propen-1-yl bicyclo[2.2.1]heptane-2-carboxylate,
Cyclopropyl bicyclo[2.2.1]heptane-2-carboxylate,
Isopropyl bicyclo[2.2.1]heptane-2-carboxylate,
Propyl bicyclo[2.2.1]heptane-2-carboxylate,
3-Buten-1-yl bicyclo[2.2.1]hept-5-ene-2-carboxylate,
Butyl bicyclo[2.2.1]hept-5-ene-2-carboxylate,
Sec-Butyl bicyclo[2.2.1]hept-5-ene-2-carboxylate,
Isobutyl bicyclo[2.2.1]hept-5-ene-2-carboxylate,
Tert-Butyl bicyclo[2.2.1]hept-5-ene-2-carboxylate,
Butyl bicyclo[2.2.1]heptane-2-carboxylate,
Sec-Butyl bicyclo[2.2.1]heptane-2-carboxylate,
Tert-Butyl bicyclo[2.2.1]heptane-2-carboxylate,
Cyclopentyl bicyclo[2.2.1]hept-5-ene-2-carboxylate,
4-Penten-1-yl bicyclo[2.2.1]hept-5-ene-2-carboxylate,
1,1-Dimethyl-2-propen-1-yl bicyclo[2.2.1]hept-5-ene-2-carboxylate,
Pentyl bicyclo[2.2.1]hept-5-ene-2-carboxylate,
3-Methylbutyl bicyclo[2.2.1]hept-5-ene-2-carboxylate,
1,1-Dimethylpropyl bicyclo[2.2.1]hept-5-ene-2-carboxylate,
3-Methyl-2-buten-1-yl bicyclo[2.2.1]heptane-2-carboxylate,
3-Methylbutyl bicyclo[2.2.1]heptane-2-carboxylate;
Bicyclo[2.2.1]hept-5-en-2-ylmethyl 3-oxobutanoate;
1-(1-Bicyclo[2.2.1]hept-5-en-2-ylethyl) 3-ethyl propanedioic acid;
1-(1-Bicyclo[2.2.1]hept-5-en-2-ylpropyl) 3-ethyl propanedioic acid;
   or any C₁₋₅alkyl esters or C₁₋₅cycloalkyl esters of any of the forgoing carboxylic acids; and all enantiomers, diastereomers, endo/exo, and E/Z isomers thereof.

In some embodiments of any of the aspects disclosed herein, particularly the second or third aspects, the compound of Formula I or the Compound of Formula II does include one or more of “Selected Compounds,” as defined herein above.

It is understood that the norbornene core of the Compounds of Formula I have the following standard alternative numberings, depending on the nature of the group Z in the following formulas:

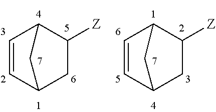

It is further understood that a norbornene core, or any compound comprising same, as drawn and/or described herein, may comprise any one or more of the following stereoisomers:

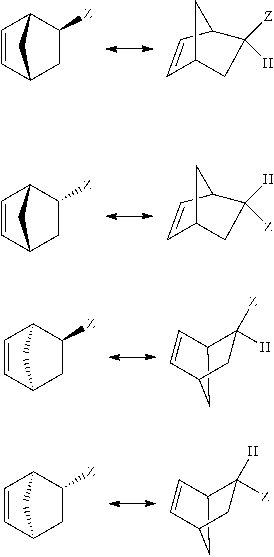

These stereoisomers are distinct from each, as shown below for the stereoisomers of 5-methoxy-2-norbornene:

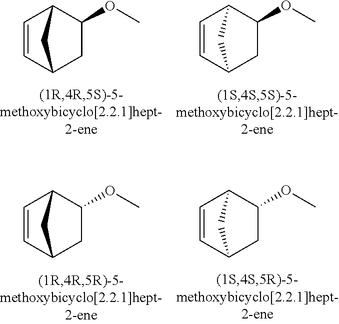

Any compound or structure referenced here may exist in any combination of these stereoisomers, for example as a racemic mixture of enantiomeric pairs, or as equal or non-equal mixtures of any diastereomers, or any other combination.

In a second aspect, the present disclosure provides a flavor composition and/or a fragrance composition (Composition 1) comprising a Compound of Formula I or II, or any of 1.1-1.210 or 2.1-2.208, optionally not including one or more of the Selected Compounds, in admixture with one or more non-toxic, orally acceptable, pharmaceutically acceptable, cosmetically acceptable, or acceptable for a household product, carriers or excipients. In particular embodiments, the second aspect provides:
1.1 Composition 1, wherein the composition is a fragrance composition.
1.2 Composition 1, wherein the composition is a flavor composition.
1.3 Composition 1, or any of Compositions 1.1-1.2, wherein the composition comprises the Compound of Formula I or II, or any of 1.1-1.210 or 2.1-2.208, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
1.4 Composition 1, or any of Compositions 1.1-1.3, wherein the composition further comprises one or more other flavors or fragrances.
1.5 Composition 1, or any of Compositions 1.1-1.4, wherein the composition further comprises one or more solvents.
1.6 Composition 1.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
1.7 Composition 1, or any of composition 1.1 to 1.6, wherein the composition is a liquid.
1.8 Composition 1, or any of compositions 1.1 to 1.6, wherein the composition is a soft or waxy solid.
1.9 Composition 1, or any of compositions 1.1-1.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
1.10 Composition 1, or any of Compositions 1.1-1.9, wherein the composition does not comprise any ingredient or component that would not be safe for ingestion, application to the oral cavity, or topical application to the skin or hair;
1.11 Composition 1, or any of Compositions 1.1-1.10, wherein the composition does not comprise any ingredient or component which is unsafe for, or not approved for, use in a food, cosmetic composition, pharmaceutical composition, oral care composition, or consumer cleaning composition.

As used herein, the term “fragrance composition” means a mixture of fragrance ingredients (e.g., including a Compound of Formula I or II or any of 1.1-1.210 or 2.1-2.208) with one or more non-toxic, cosmetically acceptable, or acceptable for a household product, carriers or excipients, such as solvents. For example, the fragrance ingredient(s) may be dissolved in a suitable solvent or mixed with a powdery substrate, with additional auxiliary substances added (e.g., additives), if desired. A fragrance composition is used, and intended to be used, to provide or impart a desired odor or aroma to a product, such as a cosmetic product or household product (e.g., household cleaners). Thus, a fragrance composition is used as an ingredient or component in a final product, such as a cosmetic product or consumer product, for which a particular fragrance is desired. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter. A fragrance composition should have enough of its fragrance ingredients so that it is effective to provide the desired odor or aroma to the final product, and this depends both on the concentration of the fragrance ingredients in the composition and the concentration of the composition used in the product.

As used herein, the term “flavor composition” means a mixture of flavor ingredients (e.g., including a Compound of Formula I or II or any of 1.1-1.210 or 2.1-2.208) with one or more non-toxic, orally acceptable, or pharmaceutically acceptable, carriers or excipients, such as solvents. For example, the flavor ingredient(s) may be dissolved in a suitable solvent or mixed with a suitable solid, semi-solid, or liquid excipients, with additional auxiliary substances added (e.g., additives), if desired. A flavor composition is used, and intended to be used, to provide or impart a desired flavor and aroma to a product, such as a food product or oral pharmaceutical product. Thus, a flavor composition is used as an ingredient or component in a final product, such as a food or oral pharmaceutical product, for which a particular flavor is desired. Examples of products having flavor compositions include, but are not limited to, oral care compositions (e.g., dental hygiene products such as mouth wash, toothpaste, floss, and breath fresheners), pharmaceutical compositions (e.g., orally administered medications including liquids, tablets or capsules), and food products. A flavor composition should have enough of its flavor ingredients so that it is effective to provide the desired flavor and aroma to the final product, and this depends both on the concentration of the flavor ingredients in the composition and the concentration of the composition used in the product.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than the Compounds of Formula I or II which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomenthyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, β-methylpropionyl, etc.), heptanoic acid esters (allyl, ethyl, octyl, propyl, methyl, etc.), heptinecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl p-methyl-p-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, P-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-napthyl methyl ether, phenyl propyl ether, p-cresyl methyl ether, vanillyl butyl ether, α-terpinyl methyl ether, citronellyl ethyl ether, geranyl ethyl ether, rose furan, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and 6-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dim ethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang lignaloe, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum, The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methyl ether; isolongifolanone; aubepine nitrile; aubepine; heliotropine; eugenol; vanillin; diphenyl oxide; hydroxycitronellal ionones; methyl ionones; isomethyl ionones; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, Persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, 6-methoxy-2,6-dimethylheptanal, 3,5-undecadien-2-one, 2,5-undecadien-1-ol, triethylthialdine. 4-methylpentyl 4-methylvalerate, (R)-N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl)propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, Stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45 (2013).

Preferred solvents and excipients for use in the compositions and products of the present disclosure include, but are not limited to, triethyl citrate, triacetin, glycerol, propylene glycol, dipropylene glycol, isopropyl myristate, ethanol, water, triglycerides, liquid waxes, propylene glycol derivatives (e.g., polymers), and ethylene glycol derivatives (e.g., polymers).

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicone.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In contrast, cosmetic excipients may include, but are not limited to, Carbopol 940 ETD, triethanolamine, purified water, glycerin, imidazolidinyl urea, EDTA, polyvinyl alcohol, methyl parabens phenoxyethanol 0, ethyl alcohol 1, peg 7 glyceryl cocoate, peg 6 triglyceryl caproic glycerides, acemulogar LANI V, isopropyl myristate, tegosoft CT, xanthan gum, sepicide CL, polyquaternium 7, and Vaseline oils. Additional suitable excipients for use with or in a flavor and/or fragrance composition for a given product will be readily selected by those having ordinary ski l in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a third aspect, the present disclosure provides a product which comprises Composition 1 or any of 1.1 to 1.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description,

The term “about,” when used to describe one of the compositions of the application, refers to a recited percentage ±5%, ±4%, ±3%, ±2.5%, ±2%, ±1.5%, ±1%, ±0.75%, ±0.5%, ±0.25%, or ±0.1%. In one embodiment, the term “about,” refers to a recited percentage ±5%. For example, “about 50%” refers to the range 45% to 55%. In one embodiment, the term “about,” refers to a recited percentage ±2.5%. In one embodiment, the term “about,” refers to a recited percentage ±1%. In one embodiment, the term “about,” refers to a recited percentage ±0.5%. In one embodiment, the term “about,” refers to a recited percentage ±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, “optionally substituted” means that the indicated core or functional group is either unsubstituted or substituted by one or more groups up to the maximum permitted by the rules of valency, wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, C₁₋₆-alkoxy, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; wherein each of said C₁₋₆alkyl, C₃₋₆cycloalkyl, heterocycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; and wherein each R^{x} is independently selected from hydrogen, C₁₋₆alkyl, C₂₋₆alkenyl, C₂₋₆alkynyl, C₃₋₆cycloalkyl, heterocycloalkyl, aryl and heteroaryl.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, sec-butyl, tert-butyl, n-pentyl, isopentyl, neopentyl, n-hexyl, and thexyl. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e., saturated linear or branched free radical consisting essentially of 1 to 3 (or 4) carbon atoms and a corresponding number of hydrogen atoms. Exemplary C₁₋₆-alkyl groups include methyl, ethyl, n-propyl, isopropyl, n-butyl, isobutyl, sec-butyl, tert-butyl. The similar terms “C₂₋₆-alkenyl,” “C₂₋₆-alkynyl,” “C₃₋₆-cycloalkyl,” “C₁₋₆-haloalkyl,” “C₁₋₆-alkoxy,” and the like, refer to corresponding functional groups having the stated number of carbon atoms, wherein “alkenyl” refers to an unsaturated linear or branched free radical having at least one double bond, “alkynyl” refers to an unsaturated linear or branched free radical having at least one triple bond, “haloalkyl” refers to an alkyl radical having at least one halogen atom attached to a carbon atom, and “alkoxy” refers to an alkyl radical having at least one oxygen atom attached to the alkyl radical and wherein the attachment point of the functional group is through the oxygen (i.e., to form an ether). Exemplary alkenyl groups include vinyl, allyl, crotyl, methallyl, and 3,3-dimethylallyl. Exemplary alkynyl groups include ethynyl and propynyl. Exemplary haloalkyl groups include fluoromethyl, difluoromethyl, trifluoromethyl, 3,3,3-trifluorethyl, and like groups with chlorine, bromine or iodine. “Cycloalkyl” refers to a carbocyclic ring attached via a ring carbon atom. Exemplary cycloalkyl groups include cyclopropyl, cyclobutyl, cyclopentyl and cyclohexyl.

As used herein, the term “heteroaryl” means an aromatic free radical having 5 to 20 atoms (i.e., ring atoms) that form a ring, wherein at least one atom (e.g., 1 to 5) of the ring atoms is carbon and at least one atom of the remaining ring atoms is a nitrogen, sulfur, or oxygen. Heteroaryl rings include monocyclic, bicyclic fused, and polycyclic fused ring systems provided that at least one ring of the ring system has at least one heteroatom (N, S, or O), and all rings are aromatic. Exemplary 5-membered heteroaryl groups include furyl, thienyl (thiophenyl), pyrrolyl, oxazolyl, thiazolyl, pyrazolyl, isothiazolyl, isoxazolyl, imidazolyl, triazolyl, oxadiazolyl, thiadiazolyl, and tetrazolyl. Exemplary 6-membered heteroaryl groups include pyridyl, pyrimidyl, pyrazinyl, pyridazinyl, and 1,2,4-triazinyl. Exemplary fused heteroaryl groups include benzoxazolyl, benzothiazolyl, benzisothiazolyl, benzisoxazolyl, benzimidazolyl, indolyl, quinolinyl, isoquinolinyl, quinazolinyl, and quinoxalinyl. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g., hetero ring atoms, can be attached to the main structure.

As used herein, the term “aryl” means an aromatic free radical having 5 or 6 atoms (i.e., ring atoms) that form a ring, wherein all of the ring atoms are carbon. Exemplary aryl groups include phenyl and naphthyl.

As used herein, the term “heterocycloalkyl” means an aromatic free radical having 3 to 20 atoms (i.e., ring atoms) that form a ring, wherein at least one atom (e.g., 1 to 5) of the ring atoms is carbon and at least one atom of the remaining ring atoms is a nitrogen, sulfur, or oxygen, and wherein at least one ring is non-aromatic. Heterocycloalkyl rings include monocyclic, bicyclic fused, bicyclic spiro-joined, polycyclic bridged, and polycyclic fused ring systems, provided that at least one ring of the ring system has at least one heteroatom (N, S, or O) and at least one ring of the ring system is non-aromatic (e.g., saturated). Exemplary saturated heterocycloalkyl groups include azetidinyl, aziridinyl, oxiranyl, oxetanyl, tetrahydrofuranyl, pyrrolidinyl, piperidinyl, piperazinyl, morpholinyl, and thiomorpholinyl. Heterocycloalkyl rings systems include ring systems in which an aromatic ring is fused to a nonaromatic ring, such as will be obtained by partial reduction of a polycyclic aromatic ring system. Exemplary ring systems of this category include indolinyl, tetrahydroquinolinyl, and tetrahydroisoquinolinyl. Other heterocycloalkyl groups will be readily apparent to those of skill in the art given the benefit of the present disclosure. A heterocycloalkyl groups can be attached to the main structure either through a carbon atom or a nitrogen atom of the ring.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### EXAMPLES

Compounds of the present disclosure can be made according to known methods published in the art.

### Synthetic Examples (Examples 1-36)

The Compounds of Examples 2, 7, 13, and 26 may be prepared according to the procedures described hereinbelow. The other compounds within the scope of the present disclosure may be made according to analogous procedure by substituting appropriate reagents as needed. Compounds may also be prepared according to known literature procedures.

¹H-NMR spectra were recorded at 300 MHz, 400 MHz, or 500 MHz on Bruker Spectrometers (Avance III HD, Avance AV-I-400, Avance AV-II-400, or 500 UltraShield). Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

GCMS and LCMS methods are as follows:
Method 1: GCMS: Instrument: Agilent 8890, G7081B 5977B MSD (EI-positive, Det. temp.: 280° C.) Mass range 50-350, Detection FID/Det. temp: 325° C., Column: Agilent DB-5MS (20 m ID 180 μm, df 0.18 μm), Average velocity: 39 cm/s, Injection vol: 1 μl, Injector temp: 250° C., Split ratio: 100/1, Carrier gas: He; Initial temp: 40° C., Initial time: 0.5 min, Solvent delay: 1.1 min, Rate 120° C./min, 115° C.; 110° C./min, 175° C.; 80° C./min, Final temp 300° C., Hold time 2.0 min.
Method 2: LCMS: Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6135B MSD (ESI pos/neg) mass range 90-1500, 1290 G7102A ELSD (Evap: 50° C., Neb: 50° C., gas flow: 1.3 ml/min), Column: XSelect CSH C18 (30×2.1 mm 3.5μ) Flow: 1 ml/min, Column temp.: 40° C., Eluent A: 0.1% Formic acid in Water, Eluent B: 0.1% Formic acid in Acetonitrile, Gradient: t=0 min 5% B, t=1.6 min 98% B, t=3 min 98% B, Postrun: 1.3 min. Source: API-ES, Capillary voltage: 3000 V, Drying gas flow: 13 L/min, Nebulizer Pressure 60 psig, Drying Gas Temp: 350° C., Fragmentor 70.
Method 3: Instrument: Agilent 6890N G1530, G2577A 5973 MSD (EI-positive, Det. temp.: 280° C.) Mass range 50-550, Column: Agilent DB-5MS (20 m ID 180 μm, df 0.18 μm), Average velocity: 50 cm/s, Injection vol: 1 μl, Injector temp: 250° C., Split ratio: 100/1, Carrier gas: He; Initial temp: 100° C., Initial time: 1.5 min, Solvent delay: 1.0 min, Rate 75° C./min, Final temp 250° C., Hold time 4.3 min.
Abbreviations: EA or EtOAc: ethyl acetate; EtOH: ethanol; MeOH: methanol; TEA: triethylamine; GC: gas chromatography; MS: mass spectrometry; THF: tetrahydrofuran; DMF: dimethylformamide; MeCN: acetonitrile; NMP: N-methylpyrrolidone; TFA: trifluoroacetic acid; DIBAL-H: diisobutylaluminum hydride; NaHMDS: sodium hexamethyldisilazide; MTBE: methyl tert-butyl ether; DCM: dichloromethane; PE: petroleum ether.

### Example 2: 2-(bicyclo[2.2.1]hept-5-en-2-yl)propanenitrile

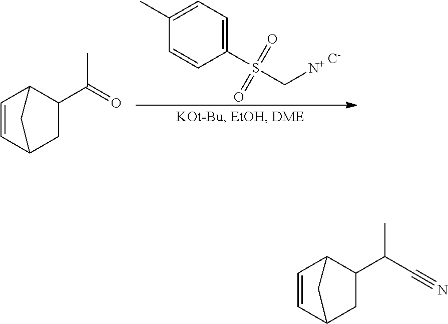

To a 500-mL round-bottom flask equipped with a magnetic stir bar is added 1-(bicyclo[2.2.1]hept-5-en-2-yl)ethan-1-one (5.0 g, 36.7 mmol, 1 equiv.), 1,2-dimethoxyethane (180 mL, 5 mL/mmol), 1-((isocyanomethyl)sulfonyl)-4-methylbenzene (TsMIC, 12.9 g, 66.1 mmol, 1.8 equiv.), and ethanol (6.0 mL, 4.74 g, 103 mmol, 2.8 equiv.). The resulting mixture is cooled below 10° C. in an ice-water bath and potassium tert-butoxide (14.0 g, 125 mmol, 3.4 equiv.) is added in portions, maintaining the internal temperature below 20° C. After the addition is complete, the mixture is allowed to warm to room temperature and it is stirred for 2 hours, with periodic monitoring by GC. After complete consumption of the ketone starting material, the reaction mixture is poured into ice-cold 1 N hydrochloric acid (300 mL) and stirred for 30 min. The resulting mixture is extracted with tert-butyl methyl ether (3×200 mL), and the combined organic layers are washed with brine (200 mL), dried over magnesium sulfate, filtered, and concentrated under reduced pressure (85 mbar, 35° C.) to afford the crude product as a dark yellow oil. Purification by column chromatography (40 g silica, gradient 0% to 30% EtOAc in hexanes over 15 min) affords the title compound (3.02 g, 56% yield, 98% GC purity) as a clear, colorless oil. GC indicates a mixture of diastereomers in a 6:4:3:3 ratio. ¹H-NMR (500 MHz, CDCl₃) δ: 6.27-6.04 (m, 2H), 3.12-2.67 (m, 3H), 2.50-1.84 (m, 2H), 1.55-1.10 (m, 5H), 0.82 (ddd, J=11.9, 4.1, 2.8 Hz, 1H). GC-MS (EI+) m/z: 147.1. This material is tested in the odor evaluation.

As an alternative procedure, potassium tert-butoxide (59.1 g, 3.4 Eq, 527 mmol) in dry THF (258 mL) is added dropwise to a stirred and solution of 2-acetyl-5-norbornene, (20.7 mL, 1 Eq, 155 mmol) and tosylmethyl isocyanide (50 g, 1.7 Eq, 0.26 mol) in a mixture of 1,2-dimethoxyethane (775 mL) and ethanol (9.05 mL, 1 Eq, 155 mmol) at 0° C. The resulting mixture is stirred at 0° C. for 30 min, then the mixture is allowed to reach room temperature and stirring is continued at that temperature for 18 h. The solution is diluted with water and extracted twice with diethyl ether, layers are separated, and the organic phase is dried over Na₂SO₄ and concentrated. The crude mixture is purified by flash column chromatography (SiO₂, 0-10% MTBE in heptane) and the obtained impure product is further purified by distillation to give 2-(bicyclo[2.2.1]hept-5-en-2-yl)propanenitrile (11.18 g, 75.94 mmol, 49% yield) as a colorless oil (27.5:19:21:32.5 mixture of isomers). GC-MS (Method 1): EI+, m/z: 147.1. ¹H NMR (400 MHz, CDCl3) δ 6.30-6.11 (m, 1H), 6.10-5.85 (m, 1H), 3.16-2.61 (m, 2H), 2.51-2.12 (m, 1H), 2.11-1.85 (m, 1H), 1.55-1.22 (m, 6H), 1.19-0.55 (m, 1H).

### Example 7: Bicyclo[2.2.1]hept-5-en-2-ylmethyl furan-2-carboxylate

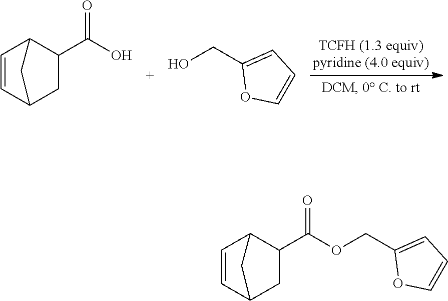 [SMILES: O=C(O)C1CC2C=CC1C2 ; OCc1ccco1 ; O=C(OCc1ccco1)C1CC2C=CC1C2]

To a 40-mL screw-cap vial equipped with a magnetic stir bar is added furan-2-carboxylic acid (750 mg, 6.69 mmol, 1.0 equiv.), bicyclo[2.2.1]hept-5-en-2-ylmethanol (914 mg, 7.36 mmol, 1.1 equiv.), dichloromethane (13.4 mL, 2 mL/mmol), and TCFH (2.63 g, 9.37 mmol, 1.4 equiv.) at room temperature. The mixture is stirred and cooled in an ice-water bath, and then pyridine (2.16 mL, 2.12 g, 26.8 mmol, 4.0 equiv.) is added, dropwise. Conversion is monitored by GC-MS. After stirring for 16 hours, the reaction mixture is partitioned between dichloromethane (20 mL) and 1 N aqueous HCl (40 mL). The aqueous phase is extracted with dichloromethane (3×30 mL), and the combined organic phases are washed with 1 N HCl (40 mL), then with brine (40 mL), dried over magnesium sulfate, filtered, and concentrated under vacuum (80 mbar, 35° C.) to afford the crude product as a brown oil. Purification by column chromatography (24 g silica, gradient 0% to 40% EtOAc in hexanes over 15 min) afforded the title compound (1.25 g, 86% yield) as a clear, colorless oil. GC indicated a mixture of diastereomers in an 8:1 ratio. ¹H-NMR (500 MHz, CDCl₃) δ: 7.74-7.44 (m, 1H), 7.18 (dd, J=9.4, 3.4 Hz, 1H), 6.50 (dd, J=3.5, 1.7 Hz, 1H), 6.18 (dd, J=5.8, 3.1 Hz, 1H), 5.99 (dd, J=5.8, 2.9 Hz, 1H), 4.08 (dd, J=10.7, 6.5 Hz, 1H), 3.89 (dd, J=10.8, 9.5 Hz, 1H), 2.95 (dq, J=3.6, 1.9 Hz, 1H), 2.62-2.41 (m, 1H), 1.89 (ddd, J=11.8, 9.2, 3.8 Hz, 1H), 1.47 (dq, J=8.3, 2.1 Hz, 1H), 1.34-1.13 (m, 2H), 0.62 (ddd, J=11.7, 4.5, 2.6 Hz, 1H). GC-MS (EI+) m/z: 218.

### Example 13: Ethyl (E)-5-(bicyclo[2.2.1]hept-5-en-2-yl)-2-methylpent-4-enoate

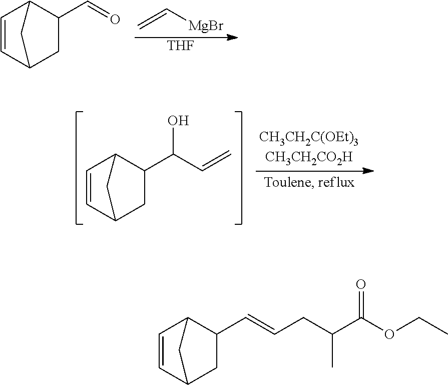

To a 2-L 3-neck round-bottom flask equipped with a magnetic stir bar and an addition funnel under nitrogen atmosphere is added vinylmagnesium bromide solution in THF (1 M, 450 mL, 450 mmol, 1.1 equiv.). The solution is cooled in an ice-water bath and bicyclo[2.2.1]hept-5-ene-2-carbaldehyde (50 g, 409 mmol, 1.0 equiv.) solution in THF (250 mL) is added dropwise via the addition funnel, maintaining the internal temperature below 20° C. After the addition is complete, the reaction mixture is allowed to warm to room temperature. Conversion is monitored by GC. After complete consumption of the aldehyde starting material (about 3 hours), the reaction mixture is cooled in an ice-water bath and quenched by the slow addition of 6 N hydrochloric acid (100 mL). The biphasic mixture is then poured into a separatory funnel and the aqueous phase is extracted with toluene (3×200 mL). The combined organic phases are washed with water (100 mL), then brine (100 mL), dried over magnesium sulfate, filtered, and concentrated under reduced pressure (30 mbar, 45° C.) to afford 1-(bicyclo[2.2.1]hept-5-en-2-yl)prop-2-en-1-ol as a brown oil in about 98% purity as indicated by GC-MS. This material is carried forward without further purification, and tested as the compound of Example 19.

The crude allylic alcohol is dissolved in toluene (400 mL) in a 2-L round-bottom flask equipped with a magnetic stir bar and a Dean-Stark trap under nitrogen atmosphere. Triethyl orthopropionate (411 mL, 360 g, 2.05 mol, 5.0 equiv.) is added, and the reaction mixture is heated to 130° C. (external temperature). Once the heating mantle has reached temperature, propionic acid (1.5 mL, 20.5 mmol, 0.05 equiv.) is added in one portion. Ethanol/toluene azeotrope begins to distill off and is periodically drained from the trap. After 2 hours, the distillation has slowed, and a further 1.5 mL propionic acid is added. Further rapid distillation is observed. This process is repeated once more, and after the third addition, no significant distillation is noted. The reaction mixture is held at 130° C. for 8 hours, and then cooled to room temperature. 2 M potassium carbonate (200 mL) is added to the reaction mixture with stirring, and then the biphasic mixture is transferred to a separatory funnel. The layers are separated, and the aqueous layer is extracted with toluene (2×100 mL). The combined organic layers are washed with brine (200 mL), dried over magnesium sulfate, filtered, and distilled (2 Torr, 109° C.) to afford the title compound (77.86 g, 332.3 mmol, 81.2%) as a clear, colorless oil. GC indicates a mixture of diastereomers in a 50:5:1:1 ratio. ¹H-NMR (500 MHz, CDCl₃) δ: 6.14-6.04 (m, 2H), 5.78-5.70 (m, 1H), 5.61-5.54 (m, 1H), 4.15-4.08 (m, 2H), 3.02-2.70 (m, 2H), 2.40-2.25 (m, 4H), 1.90-1.56 (m, 3H), 1.49-1.17 (m, 7H). GC-MS (EI+) m/z: 147.

### Example 13A: Ethyl (E)-5-(bicyclo[2.2.1]hept-5-en-2-yl)-2-methylpent-4-enoate (alternative procedure)

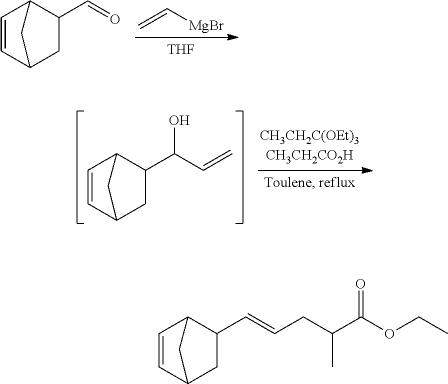

In a 50 L 4-neck-round bottom flask, to a solution of bicyclo[2.2.1]hept-5-ene-2-carbaldehyde (850 g, 6957.7 mmol, 1.0 equiv.) in THF (8.5 L) is added dropwise bromo(ethenyl)magnesium (1 M in THF, 7.6 L, 7600.0 mmol) at 0° C. under N₂ atmosphere. The reaction mixture is stirred at 0° C. for 4 h. The reaction is quenched with sat. NH₄Cl (aq.) (20 L) at 0° C. The resulting mixture is extracted with ethyl acetate (3×10 L). The combined organic layers are washed with brine (15 L), dried over anhydrous Na₂SO₄. This resulted in 1-(bicyclo[2.2.1]hept-5-en-2-yl)prop-2-en-1-ol (1010 g) as a yellow oil. GC-MS (Method 1): EI+, m/z: 150.1.

1-(Bicyclo[2.2.1]hept-5-en-2-yl)prop-2-en-1-ol (1010 g, 6457.1 mmol, 1.0 equiv.) is dissolved in toluene (10 L) at room temperature in a 20 L 4-neck round bottom flask equipped with a magnetic stir bar, a glass stopper, and a Dean-Stark trap capped with a reflux condenser, under nitrogen atmosphere. triethylorthopropionate (5925.2 g, 33617.1 mmol, 5.0 equiv.) is added and the mixture is heated to 115° C. (mantle temperature). Once the mixture reached 115° C., propanoic acid (149.4 g, 1937.2 mmol, 0.3 equiv.) is added in one portion for every 2 hours. This operation is repeated three times until no significant distillation is observed. The mixture is kept at 115° C. overnight, then cooled to room temperature. GC-MS of the reaction mixture indicates full consumption of the alcohol, and the presence of only desired product and triethyl orthopropionate. The residue is neutralized to pH 7 with saturated NaHCO₃(aq.). The resulting mixture is extracted with EA (2×10 L). The combined organic layers are washed with water (3×10 L), and dried over anhydrous Na₂SO₄. After filtration, the filtrate is concentrated under reduced pressure. The residue is purified by reversed-phase flash chromatography with the following conditions: column, C18 silica gel; mobile phase, MeCN in water (0.1% FA), 10% to 50% gradient in 10 min; detector, UV 254 nm. This resulted in ethyl 5-(bicyclo[2.2.1]hept-5-en-2-yl)-2-methylpent-4-enoate (cis/trans-1:8) (302 g, 19.17% yield) as a light yellow oil. GC-MS (Method 1): EI+, m/z: 234.1. GC indicates a mixture of diastereomers in a 73:7:1:0 ratio. ¹H NMR (300 MHz, CDCl₃) δ 6.20-6.02 (m, 1H), 6.01-5.91 (m, 1H), 5.57-5.27 (m, 1H), 5.19-5.04 (m, 1H), 4.21-3.63 (m, 2H), 2.85-2.74 (m, 2H), 2.74-2.59 (m, 1H), 2.53-2.38 (m, 1H), 2.37-2.23 (m, 1H), 2.21-1.99 (m, 1H), 1.99-1.80 (m, 1H), 1.51-1.35 (m, 1H), 1.35-1.22 (m, 4H), 1.22-1.08 (m, 3H), 0.81-0.68 (m, 1H).

### Example 26: Furan-2-ylmethyl bicyclo[2.2.1]hept-5-ene-2-carboxylate

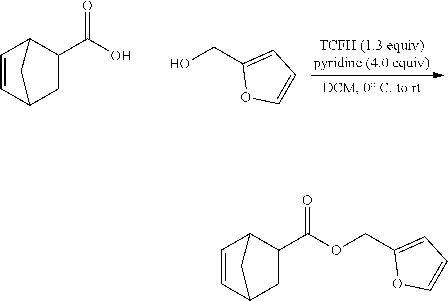 [SMILES: O=C(O)C1CC2C=CC1C2 ; OCc1ccco1 ; O=C(OCc1ccco1)C1CC2C=CC1C2]

To a 40-mL screw-cap vial equipped with a magnetic stir bar is added bicyclo[2.2.1]hept-5-ene-2-carboxylic acid (1.00 g, 7.24 mmol, 1.0 equiv.), furan-2-ylmethanol (692 μL, 781 mg, 7.96 mmol, 1.1 equiv.), dichloromethane (14.5 mL, 2 mL/mmol), and TCFH (N-(chloro(dimethylamino)methylene)-N-methylmethanaminium hexafluorophosphate) (2.64 g, 9.41 mmol, 1.3 equiv.) at room temperature. The mixture is stirred and cooled in an ice-water bath, and then pyridine (2.33 mL, 2.29 g, 29.0 mmol, 4.0 equiv.) is added, dropwise. Conversion is monitored by GC-MS. After stirring for 16 hours, the reaction mixture is partitioned between dichloromethane (20 mL) and 1 N aqueous HCl (40 mL). The aqueous phase is extracted with dichloromethane (3×30 mL), and the combined organic phases are washed with 1 N HCl (40 mL), then with brine (40 mL), dried over magnesium sulfate, filtered, and concentrated under vacuum (80 mbar, 35° C.) to afford the crude product as a brown oil. Purification by column chromatography (24 g silica, gradient 0% to 40% EtOAc in hexanes over 15 min) affords the title compound (1.39 g, 88% yield) as a clear, colorless oil. GC indicates a mixture of diastereomers in an 8:1 ratio. ¹H-NMR (500 MHz, CDCl₃) δ: 7.41 (dd, J=1.8, 0.9 Hz, 1H), 6.46-6.29 (m, 2H), 6.18 (dd, J=5.8, 3.1 Hz, 1H), 5.86 (dd, J=5.7, 2.8 Hz, 1H), 5.01 (q, J=13.1 Hz, 2H), 3.27-3.13 (m, 1H), 2.97 (dt, J=9.3, 3.9 Hz, 1H), 2.90 (dq, J=3.7, 1.8 Hz, 1H), 1.89 (ddd, J=11.8, 9.3, 3.7 Hz, 1H), 1.49-1.32 (m, 2H), 1.30-1.22 (m, 1H). GC-MS (EI+) m/z: 218.

### Example 37: Ethyl 5-(bicyclo[2.2.1]heptan-2-yl)-2-methylpent-4-enoate

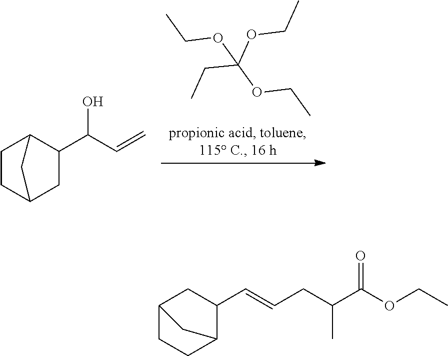 [SMILES: C=CC(O)C1CC2CCC1C2 ; CCOC(CC)(OCC)OCC ; CCOC(=O)C(C)C/C=C/C1CC2CCC1C2]

To a solution of 1-(bicyclo[2.2.1]heptan-2-yl)prop-2-en-1-ol (3.3 g, 21.7 mmol, 1.0 equiv.) (Example 62) in toluene (33 mL) is added triethyl orthopropionate (19.1 g, 108.4 mmol, 5.0 equiv.) and propanoic acid (0.5 g, 6.5 mmol, 0.3 equiv.). The reaction is stirred at 115° C. for 16 h. The reaction mixture is concentrated under reduced pressure. The residue is purified by silica gel column chromatography, eluting with PE/EA=5:1 to afford the title compound (E/Z mixture) (1.35 g, 26.35% yield) as a light yellow oil. GC-MS (Method 1): EI⁺, m/z: 236.1. ¹H NMR (300 MHz, DMSO) δ 5.63-5.12 (m, 2H), 4.04 (qd, J=7.1, 1.0 Hz, 2H), 2.48-1.90 (m, 6H), 1.76-1.61 (m, 1H), 1.56-1.38 (m, 2H), 1.37-1.22 (m, 3H), 1.17 (t, J=7.1 Hz, 3H), 1.12-0.94 (m, 4H), 0.84 (ddt, J=12.1, 5.0, 2.3 Hz, 1H).

### Example 38: Ethyl 5-(bicyclo[2.2.1]heptan-2-yl)-2-methylpentanoate

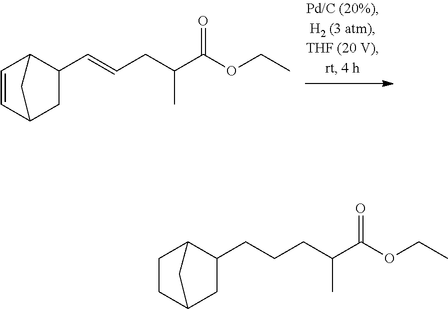 [SMILES: CCOC(=O)C(C)C/C=C/C1CC2C=CC1C2 ; CCOC(=O)C(C)CCCC1CC2CCC1C2]

To a solution of ethyl 5-(bicyclo[2.2.1]hept-5-en-2-yl)-2-methylpent-4-enoate (2.0 g, 8.5 mmol, 1.0 equiv.) in THF (40 mL) is added Pd/C (0.4 g, 20% wt). The reaction mixture is stirred at 20° C. under 3 atm of hydrogen pressure for 4 h. The reaction mixture is filtered and concentrated under reduced pressure. The residue is purified by silica gel column chromatography, eluted with PE/EA=5:1 to afford the title compound (1.3 g, 63.9% yield) as a colorless oil. GC-MS (Method 1): EI⁺, m/z: 238.1. ¹H NMR (300 MHz, CDCl₃) δ 4.15 (q, J=7.1 Hz, 2H), 2.52-2.33 (m, 1H), 2.24-1.91 (m, 2H), 1.70-1.60 (m, 2H), 1.56-1.33 (m, 4H), 1.33-1.19 (m, 9H), 1.16 (d, J=7.0 Hz, 3H), 1.12-0.87 (m, 2H), 0.69-0.49 (m, 1H).

### Example 39: Methyl (E)-5-(bicyclo[2.2.1]hept-5-en-2-yl)-2-methylpent-4-enoate

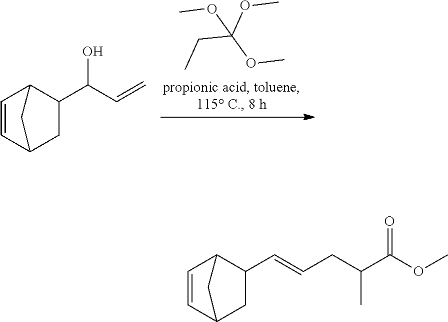

To a solution of 1-(bicyclo[2.2.1]hept-5-en-2-yl)prop-2-en-1-ol (10 g, 66.569 mmol, 1 equiv.) in toluene (100 mL) is added 1,1,1-trimethoxypropane (40.0 g, 332.8 mmol, 5.0 equiv.) and propanoic acid (1.5 g, 19.9 mmol, 0.3 equiv.). The reaction mixture is stirred at 115° C. for 8. The reaction mixture is concentrated under reduced pressure. The residue is purified by silica gel column chromatography, eluting with PE/EA=5:1 to afford the title compound (3.5 g, 23.86% yield) as a light yellow oil. GC-MS (Method 1): EI⁺, m/z: 220.0. ¹H NMR (300 MHz, CDCl3) δ 6.22-5.89 (m, 2H), 5.57-5.04 (m, 2H), 3.68 (d, J=2.4 Hz, 3H), 2.89-1.84 (m, 7H), 1.45-1.09 (m, 5H), 0.75 (ddt, J=11.6, 4.4, 2.2 Hz, 1H).

### Example 40: Methyl 5-(bicyclo[2.2.1]heptan-2-yl)-2-methylpent-4-enoate

To a solution of 1-(bicyclo[2.2.1]heptan-2-yl)prop-2-en-1-ol (3.3 g, 21.7 mmol, 1 equiv.) (Example 62) in toluene (33 mL) is added 1,1,1-trimethoxypropane (14.5 g, 108.4 mmol, 5.0 equiv.) and propanoic acid (0.5 g, 6.5 mmol, 0.3 equiv.). The reaction is stirred at 115° C. for 16. The reaction mixture is concentrated under reduced pressure. The residue is purified by silica gel column chromatography, eluting with PE/EA=5:1 to afford the title compound (E/Z mixture) (1.49 g, 30.92% yield) as a yellow oil. GC-MS (Method 1): EI⁺, m/z: 222.1. ¹H NMR (300 MHz, DMSO) δ 5.65-5.09 (m, 2H), 3.58 (s, 3H), 2.49-1.90 (m, 6H), 1.76-1.61 (m, 1H), 1.54-1.38 (m, 2H), 1.37-1.18 (m, 3H), 1.17-0.99 (m, 4H), 0.97-0.71 (m, 1H).

### Example 41: methyl 5-(bicyclo[2.2.1]heptan-2-yl)-2-methylpentanoate

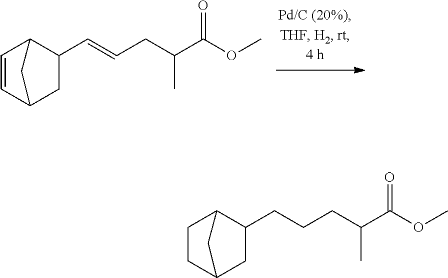 [SMILES: COC(=O)C(C)C/C=C/C1CC2C=CC1C2 ; COC(=O)C(C)CCCC1CC2CCC1C2]

To a solution of methyl (E)-5-(bicyclo[2.2.1]hept-5-en-2-yl)-2-methylpent-4-enoate (2.4 g, 10.9 mmol, 1 equiv.) (Example 39) in THF (36 mL) is added Pd/C (480 mg, 20% wt). The reaction mixture is stirred at 20° C. under 3 atm of hydrogen pressure for 4 h. The reaction mixture is filtered and concentrated under reduced pressure. The residue is purified by silica gel column chromatography, eluting with PE/EA=5:1 to afford the title compound (1.4 g, 57.28% yield) as a colorless oil. GC-MS (Method 1): EI⁺, m/z: 224.1. ¹H NMR (300 MHz, CDCl₃) δ 3.69 (s, 3H), 2.53-2.38 (m, 1H), 2.11 (d, J=18.0 Hz, 2H), 1.81-1.59 (m, 3H), 1.56-1.36 (m, 3H), 1.35-1.24 (m, 6H), 1.16 (d, J=7.0 Hz, 3H), 1.13-0.92 (m, 2H), 0.64-0.51 (m, 1H).

### Example 42: 3-(Bicyclo[2.2.1]heptan-2-yl)propanal

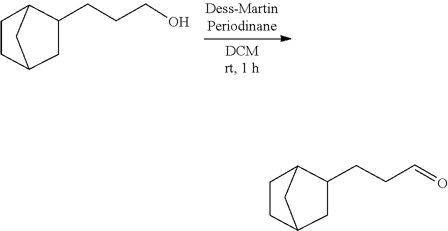 [SMILES: OCCCC1CC2CCC1C2 ; O=CCCC1CC2CCC1C2]

The general procedure of Example 50, starting from the compound of Example 43, afforded the title compound (416 mg, 36% yield) as pale yellow oil (88:12 mixture of endo/exo isomers). GC-MS (Method 1): EI⁺, m/z: 152.2. ¹H NMR (400 MHz, CDCl₃) δ 1H NMR (500 MHz, CDCl3) δ 9.76 (d, J=1.9 Hz, 1H), 2.39 (dddd, J=8.3, 6.7, 3.6, 1.6 Hz, 2H), 2.25-1.90 (m, 3H), 1.85-0.94 (m, 9H), 0.60 (dt, J=9.9, 5.0 Hz, 1H).

### Example 43: 3-(Bicyclo[2.2.1]heptan-2-yl)propan-1-ol

To a solution of ethyl 3-(bicyclo[2.2.1]heptan-2-yl)propanoate (7.0 g, 35.7 mmol, 1.0 equiv.) (Example 55) in THF (105 mL) is added DIBAL-H (1 M) (178.6 mL, 178.6 mmol, 5.0 equiv.) at −20° C. The reaction is stirred at 20° C. for 2 h. The reaction mixture is quenched by Na₂SO₄·10H₂O and stirred for 2 h at room temperature. Then, the mixture is filtered and concentrated under reduced pressure. The residue is purified by silica gel column chromatography, eluting with PE/EA=5:1 to afford the title compound (2.01 g, 36.3% yield) as a colorless oil. GC-MS (Method 1): EI⁺, m/z: 154.1. ¹H NMR (400 MHz, CDCl₃) δ 3.66 (q, J=6.6 Hz, 2H), 2.22-1.96 (m, 2H), 1.84-1.46 (m, 6H), 1.38-1.00 (m, 6H), 0.68-0.55 (m, 1H).

### Example 44: Ethyl 3-(bicyclo[2.2.1]heptan-2-yl)acrylate

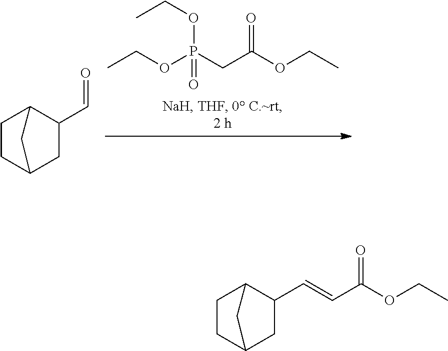

To a solution of ethyl 2-(diethoxyphosphoryl) acetate (9.9 g, 44.4 mmol, 1.1 equiv.) in THF (50 mL) is added NaH (2.4 g, 61.5 mmol, 1.5 equiv., 60%) in batches at 0° C. and the reaction is stirred at 0° C. for 1 h. A solution of bicyclo [2.2.1]heptane-2-carbaldehyde (150 mL in THF) is added dropwise to the above reaction mixture at 0° C. The reaction mixture is stirred at 25° C. for 2 h. The reaction mixture is quenched with saturated aq. NH₄Cl (500 mL), extracted with EA (3×300 mL), washed with brine (500 mL), dried with Na₂SO₄, and concentrated under reduced pressure. The residue is purified by silica gel column chromatography, eluting with PE to afford the title compound (E/Z mixture) (2 g, 25% yield) as a yellow oil. GC-MS (Method 1): EI⁺, m/z: 194.0.

### Example 45: 3-(Bicyclo[2.2.1]heptan-2-yl)prop-2-en-1-ol

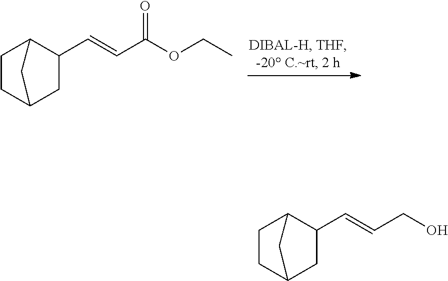

To a solution of ethyl-3-(bicyclo[2.2.1]heptan-2-yl)acrylate (E/Z mixture) (1.9 g, 9.8 mmol, 1.0 equiv.) (Example 44) in THF (38 mL) is added DIBAL-H (49.0 mL, 49.0 mmol, 5.0 equiv.) dropwise at −20° C. The reaction is stirred at room temperature for additional 2 h. The reaction mixture is quenched with NaSO₄·10H₂O and the mixture is stirred 2 h at room temperature. The mixture is filtered and the filtrate is concentrated under reduced pressure. The residue is purified by silica gel column chromatography, eluting with PE/EA=5:1 to afford the title product (E/Z mixture) (1.17 g, 79% yield) as a colorless oil. GC-MS (Method 1): EI⁺, m/z: 152.0. ¹H NMR (300 MHz, DMSO) δ 5.68-5.38 (m, 2H), 4.64-4.51 (m, 1H), 4.03-3.81 (m, 2H), 2.50-2.39 (m, 1H), 2.24-2.14 (m, 1H), 2.10 (dt, J=4.8, 2.6 Hz, 1H), 1.79-1.63 (m, 1H), 1.57-1.40 (m, 2H), 1.38-1.22 (m, 3H), 1.22-1.02 (m, 1H), 0.87 (ddd, J=12.2, 5.3, 2.3 Hz, 1H).

### Example 46: Ethyl 3-(bicyclo[2.2.1]heptan-2-yl)-2-methylacrylate

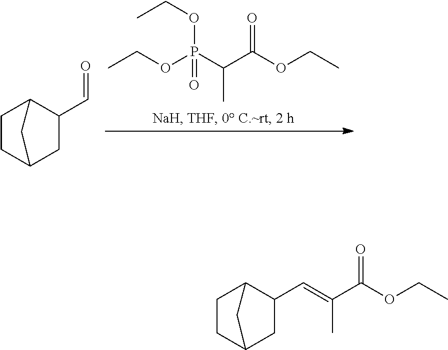

To a solution of ethyl 2-(diethoxyphosphoryl) propanoate (10.5 g, 44.4 mmol, 1.1 equiv.) in THF (100 mL) is added NaH (2.4 g, 60.5 mmol, 1.5 equiv., 60%) in batch at 0° C. The reaction mixture is stirred at 0° C. for 1 h. A solution of bicyclo[2.2.1]heptane-2-carbaldehyde (150 mL in THF) is added dropwise to the above reaction mixture at 0° C. The resulting reaction is stirred at 25° C. for 2 h. The reaction mixture is quenched with saturated aq. NH₄Cl (500 mL), extracted with EA (3×200 mL), washed with brine (300 mL), dried with Na₂SO₄, and concentrated under reduced pressure. The residue is purified by silica gel column chromatography, eluting with PE to afford the title compound (E/Z mixture) (5.0 g, 58% yield) as a yellow oil. GC-MS (Method 1): EI⁺, m/z: 208.1

### Example 47: 3-(Bicyclo[2.2.1]heptan-2-yl)-2-methylprop-2-en-1-ol

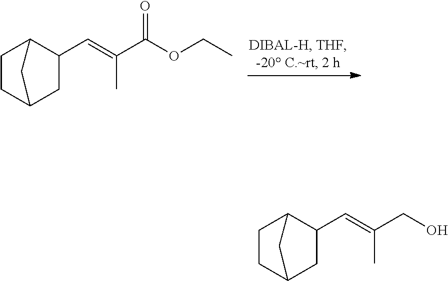

To a solution of ethyl 3-(bicyclo [2.2.1]heptan-2-yl)-2-methylacrylate (E/Z mixture) (1.9 g, 9.8 mmol, 1.0 equiv.) (Example 46) in THF (105 mL) is added DIBAL-H (49.0 mL, 49.0 mmol, 5.0 equiv.) dropwise at −20° C. The reaction is stirred at room temperature for additional 2 h. The reaction mixture is quenched with NaSO₄·10H₂O and the mixture is stirred at room temperature for 2 h. The resulting mixture is filtered and the filtrate is concentrated under reduced pressure. The residue is purified by silica gel column chromatography, eluting with PE/EA=5:1 to afford the title compound (E/Z mixture) (1.14 g, 76% yield) as a colorless oil. GC-MS (Method 1): EI⁺, m/z: 166.0. ¹H NMR (300 MHz, DMSO) δ 5.47-5.11 (m, 1H), 4.57 (dt, J=40.8, 5.6 Hz, 1H), 4.00-3.70 (m, 2H), 2.65 (ddt, J=15.9, 9.7, 5.2 Hz, 1H), 2.20-2.12 (m, 1H), 2.12-2.01 (m, 1H), 1.89-1.72 (m, 1H), 1.72-1.42 (m, 5H), 1.41-1.21 (m, 3H), 1.20-1.07 (m, 1H), 0.81-0.66 (m, 1H).

### Example 48: 3-(Bicyclo[2.2.1]hept-5-en-2-yl)but-2-en-1-ol

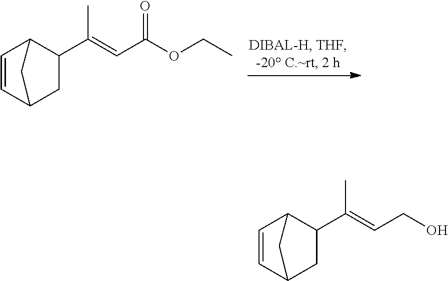

To a solution of ethyl 3-(bicyclo[2.2.1]hept-5-en-2-yl)but-2-enoate (E/Z mixture) (11 g, 53.324 mmol, 1 equiv.) (Example 56) in THF (220 mL) is added dropwise DIBAL-H (1M in THF, 266.6 mL, 266.6 mmol, 5.0 equiv.) at −20° C. The reaction is stirred at 20° C. for 2 h. The reaction is quenched with Na₂SO₄·10H₂O at 0° C. and stirred for 2 h at room temperature. The resulting mixture is filtered and concentrated under reduced pressure. The residue is purified by silica gel column chromatography, eluting with PE/EA=5:1 to afford the title compound (5.02 g, 57.32% yield) as a colorless oil. GC-MS (Method 1): EI⁺, m/z: 164.0 ¹H NMR (300 MHz, DMSO) δ 6.29-5.75 (m, 2H), 5.34-5.03 (m, 1H), 4.43 (dt, J=18.7, 5.3 Hz, 1H), 4.16-3.64 (m, 2H), 3.14-2.63 (m, 2H), 1.90-1.65 (m, 1H), 1.65-1.48 (m, 3H), 1.46-1.20 (m, 3H), 1.04-0.91 (m, 1H).

### Example 49: 3-(bicyclo[2.2.1]hept-5-en-2-yl)propan-1-ol

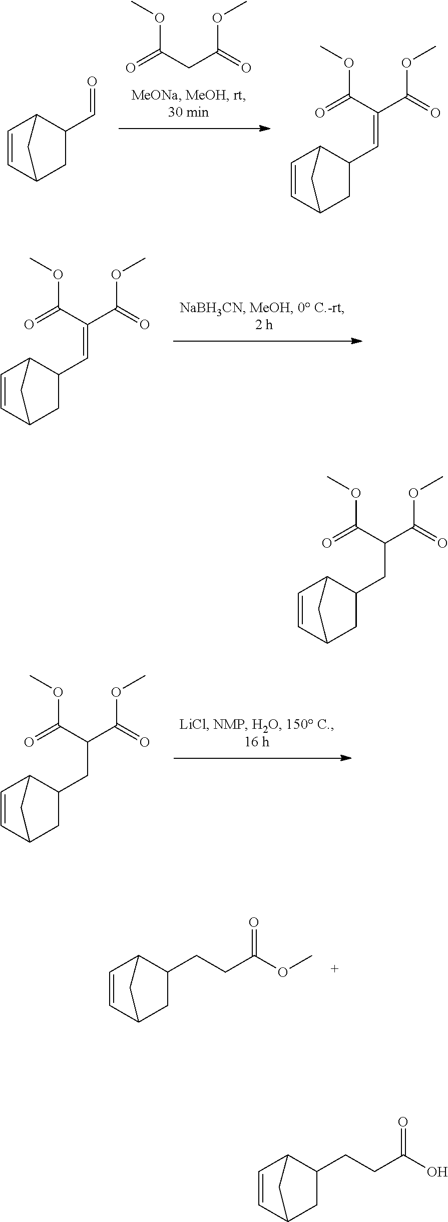

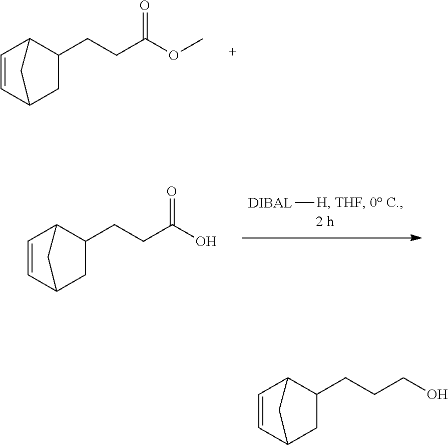

A solution of dimethyl malonate (54.1 g, 409.3 mmol, 1.0 equiv.) in MeOH (500 mL) is treated with MeONa (2.2 g, 40.9 mmol, 0.1 equiv.) at 0° C. for 10 min under nitrogen atmosphere followed by the addition of bicyclo[2.2.1]hept-5-ene-2-carbaldehyde (50 g, 409.3 mmol, 1.0 equiv.) dropwise at 0° C. The mixture is used in the next step directly.

To the above mixture is added NaBH₃CN (25.72 g, 409.28 mmol, 1.0 equiv.) in portions over 5 min at 0° C. The resulting mixture is stirred at room temperature for an additional 2 h. The reaction is quenched with water (1 L). The resulting mixture is extracted with EA (2×500 mL). The combined organic layers are washed with H₂O (1×300 mL) and brine (1×500 mL), and dried over anhydrous Na₂SO₄. After filtration, the filtrate is concentrated under reduced pressure. The residue is purified by silica gel column chromatography, eluting with PE/EA (8:1) to afford 1,3-dimethyl 2-(bicyclo[2.2.1]hept-5-en-2-ylmethyl)propanedioate (29 g, 29.74% yield) as a yellow oil, which is used in the next step directly.

A solution of 1,3-dimethyl 2-(bicyclo[2.2.1]hept-5-en-2-ylmethyl)propanedioate (29 g, 409.3 mmol, 1.0 equiv.) and LiCl (25.8 g, 608.6 mmol, 5.0 equiv.) in NMP (300 mL) and H₂O (27 mL) is stirred at 150° C. for overnight. The reaction is diluted with water (2 L). The resulting mixture is extracted with EA (2×1 L). The combined organic layers are washed with H₂O (1×1 L) and brine (1×1 L), and dried over anhydrous Na₂SO₄. After filtration, the filtrate is concentrated under reduced pressure. The residue is purified by silica gel column chromatography, eluting with PE/EA (10:1) to afford a mixture of methyl 3-(bicyclo[2.2.1]hept-5-en-2-yl)propanoate methyl 3-(bicyclo[2.2.1]hept-5-en-2-yl)propanoate and 3-(bicyclo[2.2.1]hept-5-en-2-yl)propanoic acid (18.14 g) as a yellow oil. GC-MS (Method 1): EI⁺, m/z: 180.0; 166.0.

To the mixture of methyl 3-(bicyclo[2.2.1]hept-5-en-2-yl)propanoate methyl 3-(bicyclo[2.2.1]hept-5-en-2-yl)propanoate and methyl 3-(bicyclo[2.2.1]hept-5-en-2-yl)propanoate and 3-(bicyclo[2.2.1]hept-5-en-2-yl)propanoic acid (18.14 g) in THF (200 mL), is added DIBAL-H (1M) (503.2 mL, 503.2 mmol, 5.0 equiv.) dropwise at 0° C. under nitrogen atmosphere. The reaction is stirred at room temperature for an additional 2 h. The reaction is quenched with Na₂SO₄·10H₂O at 0° C. and the mixture is stirred at room temperature for additional 2 h. The resulting mixture is filtered, and the filter cake is washed with THF (3×100 mL). The filtrate is concentrated under reduced pressure. The residue is purified by silica gel column chromatography, eluting with PE/EA (7:1) to afford the title compound (14.1 g) as a colorless oil. GC-MS (Method 1): EI⁺, m/z: 152.0. ¹H NMR (300 MHz, CDCl3) δ 6.18-5.90 (m, 2H), 3.61-3.34 (m, 2H), 2.85-2.72 (m, 2H), 2.22-1.11 (m, 8H), 0.59-0.46 (m, 1H).

### Example 50: 3-(Bicyclo[2.2.1]hept-5-en-2-yl)propanal

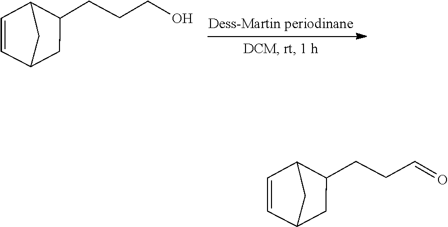 [SMILES: OCCCC1CC2C=CC1C2 ; O=CCCC1CC2C=CC1C2]

To 3-(bicyclo[2.2.1]hept-5-en-2-yl)propan-1-ol (1.50 g, 1 Eq, 9.85 mmol) (Example 49) in DCM (50 mL) is added Dess-Martin periodinane (5.01 g, 1.2 Eq, 11.8 mmol). The mixture is stirred for 1 h at room temperature. Then, the reaction is quenched with sat. Na₂SO₃, diluted with diethyl ether and the layers are separated. The organics are further washed with water and brine, combined, dried over Na₂SO₄, filtered and concentrated affording a residue. The crude product is purified by flash column chromatography (SiO₂, 0-100% MTBE in heptane), affording the title compound (517.5 mg, 3.45 mmol, 35% yield) as yellow oil (92:8 mixture of endo/exo isomers). GC-MS (Method 1): EI⁺, m/z: 150.1. ¹H NMR (400 MHz, CDCl₃) δ 9.75 (t, J=2.0 Hz, 1H), 6.15 (dd, J=5.8, 3.1 Hz, 1H), 5.93 (dd, J=5.8, 2.8 Hz, 1H), 2.77 (tt, J=3.8, 2.3 Hz, 2H), 2.42 (ddd, J=8.6, 7.0, 1.9 Hz, 2H), 2.02-1.97 (m, 1H), 1.87 (ddd, J=11.3, 9.1, 3.7 Hz, 1H), 1.45-1.39 (m, 3H), 1.23 (dt, J=8.1, 1.6 Hz, 1H), 0.52 (ddd, J=11.3, 4.3, 2.6 Hz, 1H).

### Example 51: 3-(Bicyclo[2.2.1]hept-5-en-2-yl)-2-methylpropanal

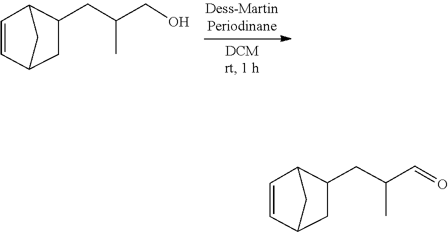

The general procedure of Example 50, starting from the compound of Example 61, afforded the title compound (875 mg, 42% yield) as pale yellow oil (90:10 mixture of endo/exo isomers). GC-MS (Method 1): EI⁺, m/z: 164.2. ¹H NMR (400 MHz, CDCl₃) δ 9.64-9.56 (m, 1H), 6.16 (dd, J=5.3, 2.6 Hz, 1H), 5.93 (dd, J=5.8, 3.0 Hz, 1H), 2.79-2.75 (m, 2H), 2.35 (hd, J=7.0, 2.4 Hz, 1H), 2.12-2.04 (m, 1H), 1.94-1.82 (m, 1H), 1.54-1.46 (m, 1H), 1.42-1.38 (m, 1H), 1.27-1.19 (m, 2H), 1.11-1.07 (m, 3H), 0.52 (dddd, J=13.8, 11.3, 4.3, 2.6 Hz, 1H).

### Example 52: 2-(Bicyclo[2.2.1]heptan-2-ylmethyl)butan-1-ol

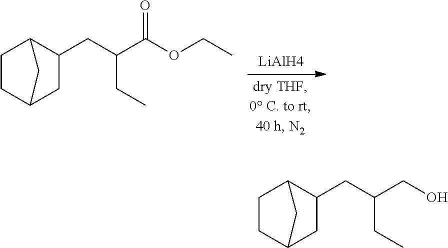

To aluminum lithium hydride in dry THF (5.0 mL, 2.4 molar, 3 Eq, 12 mmol) at 0° C. under nitrogen atmosphere is added a solution of ethyl 2-(bicyclo[2.2.1]heptan-2-ylmethyl)butanoate (0.89 g, 1 Eq, 4.0 mmol) (Example 53) in dry THF (7.9 mL) and after addition, the mixture is allowed to warm to room temperature and then stirred for 40 hours. The reaction is cooled on ice and quenched with potassium sodium tartrate solution (26 mL, 1.5 molar, 10 Eq, 40 mmol), stirring until gas formation ceased. Then the mixture is diluted with EtOAc and the layers are separated. The aqueous layer is extracted twice more with EtOAc, the organics are combined, washed with water, dried over Na₂SO₄, filtered and evaporated under reduced pressure. The crude mixture is purified by flash column chromatography (SiO₂, 0-40% MTBE in heptane) to afford the title compound (610 mg, 3.35 mmol, 84%) as a colorless oil (90:10 mixture of isomers). GC-MS (Method 1): EI⁺, m/z: 182.1. ¹H NMR (400 MHz, CDCl3) δ 3.80-3.48 (m, 2H), 2.21-2.03 (m, 2H), 1.94-1.81 (m, 1H), 1.80-1.69 (m, 1H), 1.57-0.97 (m, 12H), 0.96-0.84 (m, 3H), 0.63-0.53 (m, 1H).

### Example 53: Ethyl 2-(bicyclo[2.2.1]heptan-2-ylmethyl)butanoate

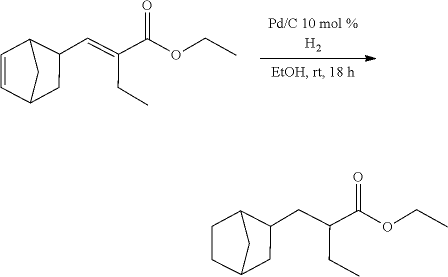 [SMILES: CCOC(=O)/C(=C/C1CC2C=CC1C2)CC ; CCOC(=O)C(CC)CC1CC2CCC1C2]

A stirred solution of ethyl 2-(bicyclo[2.2.1]hept-5-en-2-ylmethylene)butanoate (2.17 g, 1 Eq, 9.85 mmol) (Example 57) in EtOH (40 mL) is purged with nitrogen for 5 min. Palladium, 10% on activated carbon, (1.05 g, 5% Wt., 0.05 Eq, 492 μmol) is added and the solution is purged with nitrogen for another 5 min. The atmosphere is replaced by hydrogen, by submitting the mixture to three cycles of vacuum/hydrogen. The mixture is stirred at room temperature for 18 h. The reaction is filtered over celite and the filter cake is rinsed twice with EtOH, then the filtrate is concentrated. The crude product is purified by flash column chromatography (SiO₂, 0-20% MTBE in heptane) to afford the title compound (0.89 g, 4.0 mmol, 40% yield) as a colorless oil (89:11 mixture of isomers). GC-MS (Method 1): EI⁺, m/z: 224.1. ¹H NMR (400 MHz, CDCl₃) δ 4.13 (qd, J=7.1, 2.3 Hz, 2H), 2.28-2.20 (m, 1H), 2.19-2.02 (m, 2H), 1.78-1.57 (m, 3H), 1.56-1.36 (m, 3H), 1.36-1.21 (m, 7H), 1.14-1.00 (m, 1H), 0.89 (tt, J=7.5, 2.4 Hz, 4H), 0.57 (tt, J=10.1, 3.1 Hz, 1H).

### Example 54: Ethyl 3-(bicyclo[2.2.1]heptan-2-yl)-2-methylpropanoate

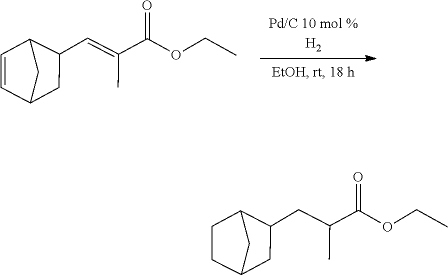

A stirred solution of ethyl 3-(bicyclo[2.2.1]hept-5-en-2-yl)-2-methylacrylate (1.00 g, 1 Eq, 4.85 mmol) (Example 58) in EtOH (25 mL) is purged with nitrogen for 5 min. Palladium, 10% on activated carbon, (103 mg, 5% Wt., 0.01 Eq, 48.5 μmol) is added and the solution is purged with nitrogen for another 5 min. The atmosphere is replaced by hydrogen, by submitting the mixture to three cycles of vacuum/hydrogen. The mixture is stirred at room temperature for 18 h. The reaction is filtered over celite, and the filter cake is rinsed with EtOH. The filtrate is concentrated. The crude product is purified by flash column chromatography (SiO₂, 0-100% MTBE in heptane) to afford the title compound (818.3 mg, 3.891 mmol, 80.3%) as a colorless oil (mixture of isomers). GC-MS (Method 1): EI⁺, m/z: 210.0. ¹H NMR (400 MHz, CDCl₃) δ 4.12 (qd, J=7.1, 1.8 Hz, 2H), 2.48-2.33 (m, 1H), 2.20-2.04 (m, 2H), 1.81-1.67 (m, 3H), 1.48 (ddddd, J=14.5, 12.5, 6.3, 4.6, 2.3 Hz, 2H), 1.42-1.28 (m, 3H), 1.25 (t, J=7.1 Hz, 4H), 1.14 (dd, J=6.9, 1.4 Hz, 3H), 1.12-1.05 (m, 1H), 0.66-0.53 (m, 1H).

### Example 55: Ethyl 3-(bicyclo[2.2.1]heptan-2-yl)propanoate

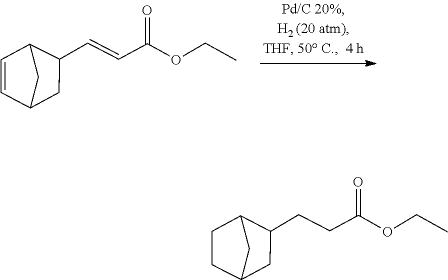

To a solution of ethyl (E)-3-(bicyclo[2.2.1]hept-5-en-2-yl)acrylate (Example 59) (E/Z mixture) (10 g, 10.894 mmol, 1 equiv.) in THF (150 mL) is added Pd/C (2 g 20% wt). The reaction mixture is stirred at 50° C. under 20 atm of hydrogen atmosphere for 4 h. The reaction mixture is filtered and concentrated under reduced pressure. The residue is purified by silica gel column chromatography, eluting with PE/EA=5:1 to afford the title compound (7.0 g, 68.6% yield) as a colorless oil. GC-MS (Method 1): EI⁺, m/z: 196.1. ¹H NMR (400 MHz, CDCl₃) δ 3.66 (q, J=6.6 Hz, 2H), 2.22-1.96 (m, 2H), 1.84-1.46 (m, 6H), 1.38-1.00 (m, 6H), 0.68-0.55 (m, 1H).

### Example 56: Ethyl 3-(bicyclo[2.2.1]hept-5-en-2-yl)but-2-enoate

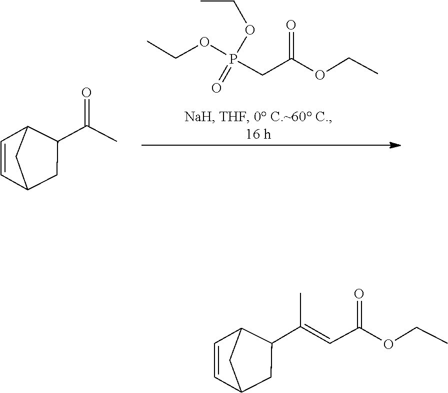

To a solution of ethyl 2-(diethoxyphosphoryl) acetate (32.9 g, 147.0 mmol, 2.0 equiv.) in THF (200 mL) is added NaH (5.9 g, 147.0 mmol, 2.0 equiv., 60%) in batches at 0° C., then the reaction mixture is stirred at 0° C. for 1 h. Then 1-(bicyclo[2.2.1]hept-5-en-2-yl)ethan-1-one (10 g, 73.5 mmol, 1.0 equiv.) is added dropwise at 0° C. The reaction mixture is stirred at 60° C. for 15 h. The reaction mixture is quenched with saturated aq. NH₄Cl (300 mL), extracted with EA (3×200 mL), dried with Na₂SO₄, and concentrated under reduced pressure. The residue is purified by silica gel column chromatography, eluting with PE to afford the title product (E/Z mixture) (11.1 g, 73.28% yield) as a yellow oil. GC-MS (Method 1): EI⁺, m/z: 206.0. ¹H NMR (400 MHz, CDCl3) δ 6.28-5.82 (m, 2H), 573-5.45 (m, 1H), 4.20-4.06 (m, 2H), 3.12-2.77 (m, 2H), 2.23-2.18 (m, 3H), 2.10-1.80 (m, 1H), 1.55-1.05 (m, 7H).

### Example 57: ethyl 2-(bicyclo[2.2.1]hept-5-en-2-ylmethylene)butanoate

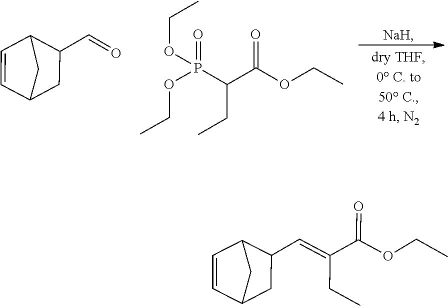 [SMILES: O=CC1CC2C=CC1C2 ; CCOC(=O)C(CC)P(=O)(OCC)OCC ; "NaH," ; CCOC(=O)/C(=C/C1CC2C=CC1C2)CC]

To sodium hydride (794 mg, 60% Wt., 1.2 Eq, 19.8 mmol) in dry THF (10 mL) at 0° C. is added under N2 atmosphere a solution of ethyl 2-(diethoxyphosphoryl)butanoate (5.00 g, 1.2 Eq, 19.8 mmol) in dry THF (20 mL) slowly dropwise. The mixture is allowed to warm to room temperature and stirred for 30 min before addition of a solution of bicyclo[2.2.1]hept-5-ene-2-carbaldehyde (1.96 mL, 1 Eq, 16.5 mmol) in dry THF (15 mL). The reaction is stirred at room temperature for 1 h and then 3 h at 50° C. The solution is diluted with water and extracted with diethyl ether, the layers are separated, and the aqueous layer is further extracted with diethyl ether twice. The organics are combined, washed with brine and then dried over Na₂SO₄, filtered and evaporated under reduced pressure. The crude mixture is purified by flash column chromatography (SiO₂, 0-50% MTBE in heptane) to afford the title compound (2.17 g, 9.2 mmol, 55% yield) as colorless oil (70:10:20 mixture of isomers). GC-MS (Method 1): EI⁺, m/z: 220.1.

### Example 58: Ethyl (E)-3-(bicyclo[2.2.1]hept-5-en-2-yl)-2-methylacrylate

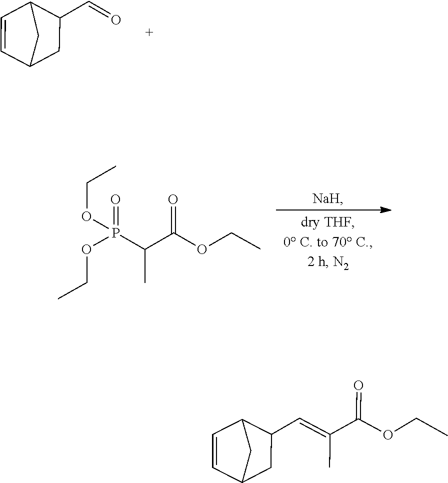

To sodium hydride (1.96 g, 60% Wt., 1.2 Eq, 49.1 mmol) in dry THF (13.6 mL) at 0° C. is added under N₂ atmosphere a solution of triethyl-2-phosphonopropionate (10.6 mL, 1.2 Eq, 49.1 mmol) in dry THF (13.6 mL) slowly dropwise. The mixture is allowed to warm to room temperature and stirred for 30 min, then a solution of 5-norbornene-2-carboxaldehyde (4.87 mL, 1 Eq, 40.9 mmol) in dry THF (13.6 mL) is added and the reaction is refluxed at 70° C. for 2 hours. The solution is diluted with water and extracted with diethyl ether, the layers are separated, and the organic phase is dried over Na₂SO₄ and concentrated. The crude mixture is purified by flash column chromatography (SiO₂, 0-10% MTBE in heptane) to give the title compound as a colorless oil (1:1 mixture of endo E/Z isomers, contains traces of exo E/Z mixture). GC-MS (Method 1): EI⁺, m/z: 206.1. ¹H NMR (400 MHz, CDCl₃) δ 6.34 (dq, J=9.9, 1.5 Hz, 1H, E isomer), 6.22 (ddd, J=8.9, 5.7, 3.1 Hz, 2H), 6.00 (ddd, J=11.3, 5.8, 2.9 Hz, 2H), 5.49 (dq, J=10.0, 1.5 Hz, 1H, Z isomer), 4.24-4.19 (m, 2H, E isomer), 4.18-4.13 (m, 2H, Z isomer), 3.60-3.53 (m, 1H, Z isomer), 3.00-2.93 (m, 1H, E isomer), 2.92-2.89 (m, 1H, Z isomer), 2.88-2.87 (m, 2H, E isomer), 2.86-2.82 (m, 1H, Z isomer), 2.05 (ddt, J=12.1, 9.2, 3.4 Hz, 2H), 1.88 (d, J=1.5 Hz, 3H, E isomer), 1.84 (d, J=1.4 Hz, 3H, Z isomer), 1.43 (ddtd, J=20.3, 8.6, 4.4, 1.8 Hz, 3H), 1.34-1.24 (m, 7H), 0.82 (ddd, J=11.7, 4.2, 2.5 Hz, 1H, E isomer), 0.72 (ddd, J=11.7, 4.2, 2.5 Hz, 1H, Z isomer).

### Example 59: Ethyl-3-(bicyclo[2.2.1]hept-5-en-2-yl)acrylate

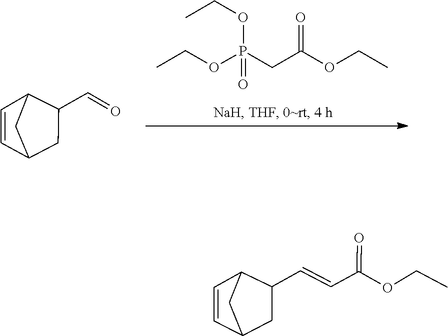

To a solution of ethyl 2-(diethoxyphosphoryl) acetate (21.2 g, 94.5 mmol, 1.05 equiv.) in THF (200 mL) is added NaH (4.68 g, 117.1 mmol, 1.3 equiv., 60%) in batches at 0° C. The reaction mixture is stirred at 0° C. for 1 h. Then to the mixture is added bicyclo[2.2.1]hept-5-ene-2-carbaldehyde (10 g, 73.4 mmol, 1 equiv.) dropwise at 0° C. The resulting reaction mixture is stirred at 60° C. for 15 h. The reaction mixture is quenched by saturated aq. NH₄Cl (200 mL), extracted with EA (3×100 mL), dried with Na₂SO₄, and concentrated under reduced pressure. The residue is purified by silica gel column chromatography, eluting with PE to afford the title compound (E/Z mixture) (10 g, 57.8% yield) as a yellow oil. GC-MS (Method 1): EI⁺, m/z: 192.1. ¹H NMR (300 MHz, CDCl₃) δ 7.04-6.54 (m, 1H), 6.25-6.10 (m, 1H), 6.03-5.93 (m, 1H), 5.87-5.71 (m, 1H), 4.23-4.11 (m, 2H), 294-2.78 (m, 2H), 2.02-1.92 (m, 1H), 1.50-1.35 (m, 1H), 1.34-1.22 (m, 5H), 0.95-0.85 (m, 1H).

### Example 60: Ethyl 3-(bicyclo[2.2.1]hept-5-en-2-yl)-2-methylpropanoate

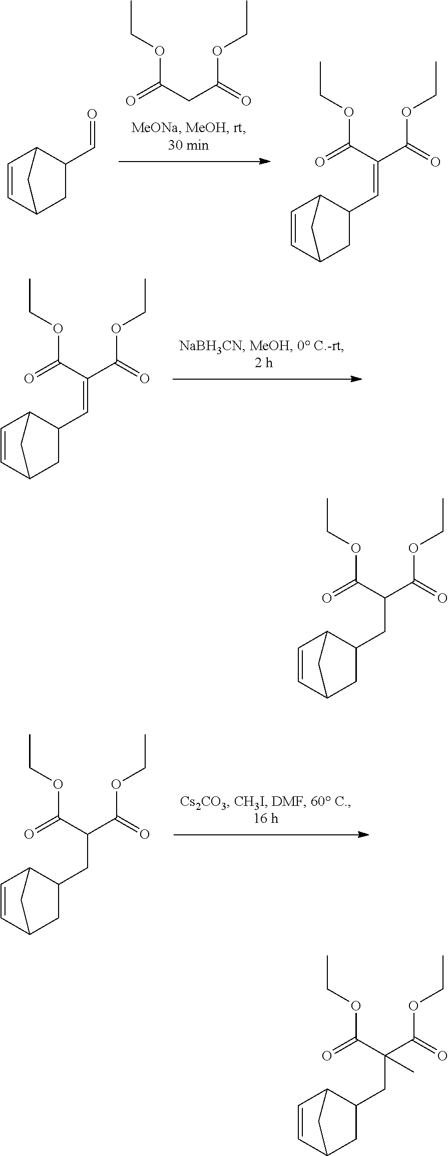

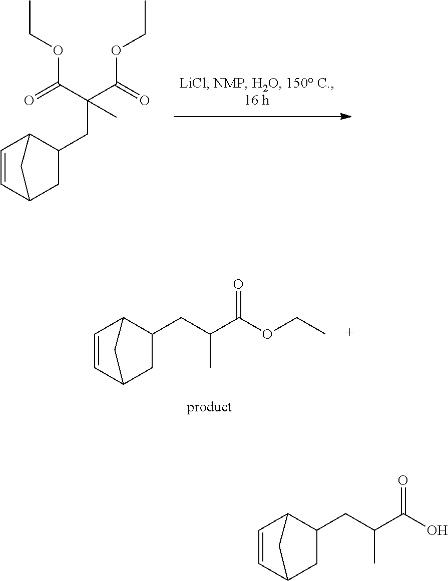 [SMILES: CCOC(=O)C(C)(CC1CC2C=CC1C2)C(=O)OCC ; CCOC(=O)C(C)CC1CC2C=CC1C2 ; CC(CC1CC2C=CC1C2)C(=O)O]

A solution of diethyl malonate (262.2 g, 1637.1 mmol, 1.0 equiv.) and MeONa (8.8 g, 163.7 mmol, 0.1 equiv.) in MeOH (2 L) is stirred at 0° C. for 5 min under air atmosphere. To this mixture is added bicyclo[2.2.1]hept-5-ene-2-carbaldehyde (200.0 g, 1637.1 mmol, 1.0 equiv.) in portions over 10 min at room temperature. The resulting mixture is stirred at room temperature for an additional 30 min. The resulting mixture is concentrated under reduced pressure. The crude product (255.0 g) is used in the next step directly without further purification. GC-MS (Method 1): EI⁺, m/z: 264.0.

To a solution of 1,3-diethyl 2-(bicyclo[2.2.1]hept-5-en-2-ylmethylidene)propanedioate (255.0 g, 964.7 mmol, 1.0 equiv., crude) in MeOH (2.5 L) is added NaBH₃CN (60.6 g, 964.7 mmol, 1.0 equiv.), and the reaction is stirred at room temperature for 2 h under air atmosphere. The reaction is poured into sat. NH₄Cl (aq.) (4 L) at room temperature. The resulting mixture is extracted with EA (3×3 L). The combined organic layers are washed with water (3 L), brine (3 L), and dried over anhydrous Na₂SO₄. After filtration, the filtrate is concentrated under reduced pressure. The residue is purified by silica gel column chromatography, eluting with PE/THF (5:1) to afford 1,3-diethyl 2-(bicyclo[2.2.1]hept-5-en-2-ylmethyl)propanedioate (208 g, 80.95% yield) as a yellow oil. GC-MS (Method 1): EI⁺, m/z: 266.0.

A solution of 1,3-diethyl 2-(bicyclo[2.2.1]hept-5-en-2-ylmethyl)propanedioate (208 g, 781.0 mmol, 1.0 equiv.) in DMF (2 L) is treated with Cs₂CO₃ (381.7 g, 1171.5 mmol, 1.5 equiv.) at room temperature for 10 min under nitrogen atmosphere followed by the addition of CH₃I (554.3 g, 3904.8 mmol, 5.0 equiv.) dropwise at 0° C. The resulting mixture is stirred at 60° C. overnight under air atmosphere. The precipitated solids are removed by filtration and washed with ethyl ether (3×1 L). The resulting mixture is extracted with EA (3×2 L). The combined organic layers are washed with water (6×2 L), and dried over anhydrous Na₂SO₄. After filtration, the filtrate is concentrated under reduced pressure. The residue is purified by reversed-phase flash chromatography with the following conditions: column, C18 silica gel; mobile phase, MeCN in Water (0.1% TFA), 10% to 50% gradient in 10 min; detector, UV 254 nm. This resulted in 1,3-diethyl 2-(bicyclo[2.2.1]hept-5-en-2-ylmethyl)-2-methylpropanedioate (95.5 g, 43.62% yield, 95% purity) as a yellow oil. GC-MS (Method 1): EI⁺, m/z: 280.0.

A mixture of 1,3-diethyl 2-(bicyclo[2.2.1]hept-5-en-2-ylmethyl)-2-methylpropanedioate (95.5 g, 338.8 mmol, 1.0 equiv.) and LiCl (72.2 g, 1703.2 mmol, 1.0 equiv.) in NMP (900 mL) and H₂O (100 mL) is stirred at 150° C. for overnight. The reaction is diluted with water at room temperature. The resulting mixture is extracted with EA (3×3 L). The combined organic layers are washed with sat. NH₄Cl (aq.) (3×3 L), and dried over anhydrous Na₂SO₄. After filtration, the filtrate is concentrated under reduced pressure. The residue is purified by reversed-phase flash chromatography with the following conditions: column, C18 silica gel; mobile phase, MeCN in Water (0.1% TFA), 10% to 50% gradient in 10 min; detector, UV 254 nm. This resulted in the title compound (ethyl 3-(bicyclo[2.2.1]hept-5-en-2-yl)-2-methylpropanoate) (25.47 g) as a yellow oil, and 3-(bicyclo[2.2.1]hept-5-en-2-yl)-2-methylpropanoic acid (20 g) as a yellow oil. GC-MS (Method 1): EI⁺, m/z: 208; 180.0.

### Example 61: 3-(Bicyclo[2.2.1]hept-5-en-2-yl)-2-methylpropan-1-ol

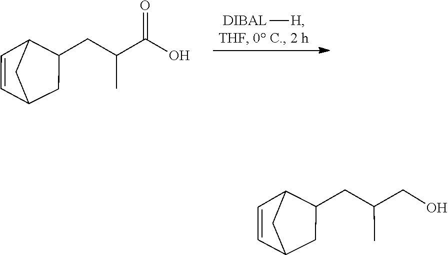

To a stirred solution of 3-(bicyclo[2.2.1]hept-5-en-2-yl)-2-methylpropanoic acid (20 g, 110.96 mmol, 1.0 equiv.) (Example 60, by-product acid) in THF (400 mL) is added DIBAL-H (1M) (554.8 mL, 554.8 mmol, 5.0 equiv.) dropwise at 0° C. under nitrogen atmosphere. The reaction is quenched with the addition of Na₂SO₄·10H₂O at 0° C. and the reaction is stirred at room temperature for 2 h. The resulting mixture is filtered, and the filter cake is washed with THF (3×100 mL). The filtrate is concentrated under reduced pressure. The residue is purified by silica gel column chromatography, eluting with PE/EA (8:1) to afford the title compound (12.1 g, 65.59% yield) as a yellow oil. GC-MS (Method 1): EI⁺, m/z: 166.0. ¹H NMR (300 MHz, CDCl₃) δ 6.23-5.86 (m, 2H), 3.61-3.34 (m, 2H), 2.85-2.43 (m, 2H), 2.22-2.05 (m, 1H), 1.95-1.80 (m, 1H), 1.78-1.42 (m, 1H), 1.42-1.30 (m, 3H), 1.29-1.21 (m, 1H), 1.20-0.86 (m, 5H), 0.59-0.46 (m, 1H). This material is used in odor testing.

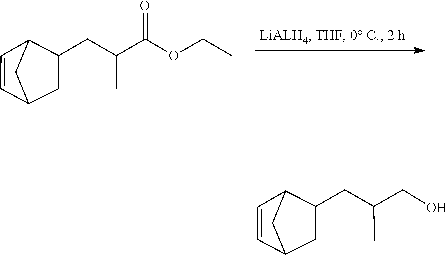

Alternatively, the title compound can be prepared from the Example 60 ester. To a stirred solution of ethyl 3-(bicyclo[2.2.1]hept-5-en-2-yl)-2-methylpropanoate (25.47 g, 122.2 mmol, 1.0 equiv.) (Example 60) in THF (250 mL) is added LiAlH₄ (2M) (152.9 mL, 305.7 mmol, 2.5 equiv.) dropwise at 0° C. under nitrogen atmosphere. After the reaction completed, the reaction is quenched with Na₂SO₄·10H₂O at 0° C. The resulting mixture is filtered, and the filter cake is washed with THF (3×100 mL). The filtrate is concentrated under reduced pressure. The residue is purified by silica gel column chromatography, eluting with PE/EA (8:1) to afford the title compound (18.1 g, 89.99% yield) as a yellow oil. GC-MS (Method 1): EI⁺, m/z: 166.0.

### Example 62: 1-(Bicyclo[2.2.1]heptan-2-yl)prop-2-en-1-ol

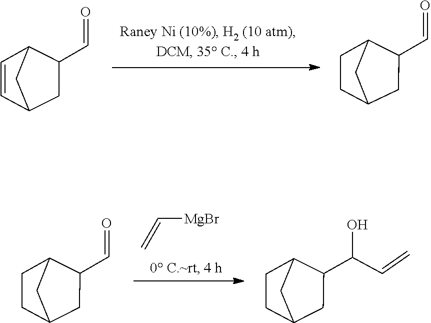

To a solution of bicyclo[2.2.1]hept-5-ene-2-carbaldehyde (10 g, 81.855 mmol, 1.0 equiv.) in DCM (300 mL) is added Raney-Ni (1 g, 10% wt.), the mixture is stirred at 35° C. under 10 atm of hydrogen pressure for 4 h. The reaction solution is filtered, and the filtrate is used in the next step directly. GC-MS (Method 1): EI⁺, m/z: 124.0

Vinylmagnesium bromide (1 M) (90.0 mL, 90.0 mmol, 1.1 equiv.) is added dropwise to the reaction mixture obtained in previous step under N₂ at 0° C. The reaction mixture is stirred at 20° C. for 4 h. The mixture is quenched by saturated aq. NH₄Cl (200 mL), extracted with DCM (3×200 mL), dried with Na₂SO₄, and concentrated under reduced pressure. The residue is purified by silica gel column chromatography, eluting with PE/THF (10/1) to afford the title compound (7 g, 56.2% yield) as a light yellow oil. GC-MS (Method 1): EI⁺, m/z: 152.1.

### Example 63: 2-(methylbicyclo[2.2.1]hept-5-en-2-yl)methanol

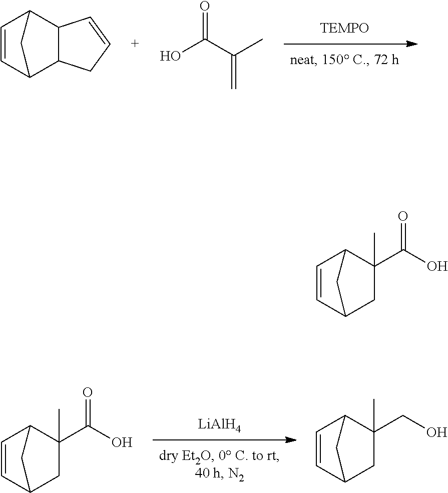

A 50 mL reaction tube is charged with dicyclopentadiene (7.5 mL, 0.8 Eq, 56 mmol) and methacrylic acid (5.9 mL, 1 Eq, 70 mmol), followed by TEMPO (22 mg, 0.002 Eq, 0.14 mmol). The reaction mixture is stirred in the closed reaction tube at 150° C. for 72 h. The reacting mixture is allowed to reach room temperature. The crude oil is treated with diethyl ether and the solids formed are filtered off over a glass filter. The solids are further washed with diethyl ether and the filtrate is evaporated affording a thick orange oil. The crude oil is purified by flash column chromatography (SiO₂, 0-25% ethyl acetate in heptane) affording in order of elution exo-2-methylbicyclo [2.2.1]hept-5-ene-2-carboxylic acid (3.18 g, 19 mmol, 28% yield) as a white thick solid, and 2-methylbicyclo[2.2.1]hept-5-ene-2-carboxylic acid (6.4 g, 41 mmol, 60% yield) as a colorless glassy solid (2:1 mixture of endo/exo isomers). Reported characterization refers to the 2:1 mixture of isomers. GC-MS (Method 1): EI⁺, m/z, 152.0. ¹H NMR (400 MHz, CDCl₃) δ 6.23 (dd, J=5.6, 3.0 Hz, 0.5H, exo), 6.17 (dd, J=5.7, 2.9 Hz, 1H, endo), 6.13 (dd, J=5.7, 2.8 Hz, 1H, endo), 6.10 (dd, J=5.6, 3.2 Hz, 0.5H, exo), 3.08-3.02 (m, 0.5H, exo), 2.84 (s, 1.5H), 2.79 (t, J=2.4 Hz, 1H, endo), 2.43 (dd, J=12.1, 3.9 Hz, 0.5H, exo), 1.88 (dd, J=12.0, 2.6 Hz, 1H, endo), 1.57 (d, J=8.8 Hz, 1H), 1.49 (s, 3H, endo), 1.46 (ddd, J=7.2, 5.5, 3.6 Hz, 3H), 1.16 (s, 1.5H, exo), 0.91-0.84 (m, 0.5H, exo).

2-methylbicyclo[2.2.1]hept-5-ene-2-carboxylic acid (5.18 g, 1 Eq, 34.0 mmol, 2:1 mixture endo/exo) is dissolved in dry diethyl ether (170 mL) under nitrogen atmosphere, then the resulting mixture is cooled down to 0° C. To this, lithium aluminum hydride in dry THF (21.3 mL, 2.4 molar, 1.5 Eq, 51.1 mmol) is added dropwise over a period of 10 min. A thick white suspension started to form. The reaction is stirred at 0° C. for 1 h, then allowed to stir for 40 h at room temperature. After reaction completion, the reaction mixture is cooled down again to 0° C. and slowly quenched by addition of water, then diluted with a saturated solution of potassium sodium tartrate (Rochelle's salt) and stirred at room temperature for 1 h. The resulting suspension is diluted with diethyl ether and the layers are separated. The aqueous layer is extracted thrice more with diethyl ether. The combined organics are washed with brine, dried over Na₂SO₄, filtered and evaporated under reduced pressure, to afford the title compound (5.35 g, 32 mmol, 93% yield) as a colorless oil (1:0.8 mixture of isomers).

GC-MS (Method 1): EI⁺, m/z: 120.1 [fragment mass], calcd. 138.2. LCMS (Method 2): M+H=139.2; calcd. 139.210.

### Example 64: 2-(Methylbicyclo[2.2.1]heptan-2-yl)methanol

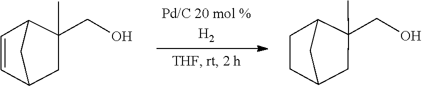

A stirred solution of 2-(methylbicyclo[2.2.1]hept-5-en-2-yl)methanol (1.5 g, 1 Eq, 11 mmol) (Example 63) in THF (54 mL) is purged with nitrogen for 5 min. Palladium, 10% on activated carbon (50% wet with water) (0.46 g, 5% Wt., 0.02 Eq, 0.22 mmol) is added and the solution is purged with nitrogen for another 5 min. The atmosphere is replaced by hydrogen, by submitting the mixture to three cycles of vacuum/hydrogen, and the resulting mixture is stirred at room temperature for 2 h. After reaction completion, the atmosphere is purged with nitrogen for 5 min. The mixture is then filtered off over a plug of celite and the filter cake is washed thrice with ethanol and evaporated under reduced pressure, affording the title compound as a colorless oil (60:40 mixture of isomers). GC-MS (Method 1): EI⁺, m/z: 140.2. ¹H NMR (400 MHz, CDCl₃) δ 3.51 (dd, J=10.8, 5.0 Hz, 0.6H, endo), 3.40 (dt, J=10.8, 6.2 Hz, 1H), 3.22 (dd, J=10.6, 5.6 Hz, 0.4H, exo), 2.20 (q, J=4.5 Hz, 1H), 1.99 (d, J=4.0 Hz, 0.4H, exo), 1.94 (d, J=3.9 Hz, 0.6H, endo), 1.68-1.48 (m, 3H), 1.37 (dddd, J=13.7, 8.3, 6.3, 3.1 Hz, 1H), 1.30-1.22 (m, 1H), 1.21-1.15 (m, 2H), 1.04 (d, J=1.5 Hz, 4H), 0.84 (ddd, J=13.5, 10.9, 2.7 Hz, 1H).

### Example 65: 2-(Methylbicyclo[2.2.1]heptan-2-yl)acetonitrile

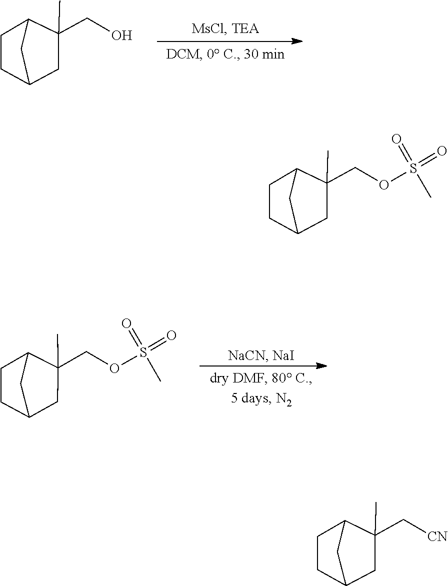

To a solution of 2-(methylbicyclo[2.2.1]heptan-2-yl)methanol (1.49 g, 92% Wt., 1 Eq, 9.78 mmol) (Example 64) in DCM (40 mL) is added TEA (2.2 mL, 1.6 Eq, 15.6 mmol) under nitrogen atmosphere and the reacting mixture is then cooled down to 0° C. To the cold solution, methanesulfonyl chloride (908 μL, 1.2 Eq, 11.7 mmol) is slowly added dropwise via syringe over a period of 5 min. The mixture is left stirring at 0° C. temperature for 30 min. After reaction completion, the mixture is slowly quenched with ice brine/water 1:1 solution (50 mL) and the layers are separated. The aqueous layer is extracted twice more with DCM and then the organics are combined, washed with brine, dried over Na₂SO₄, filtered and evaporated under reduced pressure, affording 2-(methylbicyclo[2.2.1]heptan-2-yl)methane methanesulfonate (2.56 g, 9.1 mmol, 94% yield) as a brown oil (65:35 mixture of isomers, based on GCMS). The oil is used without purification directly in the next step. GCMS (Method 3): 109.2 [fragment mass], calcd. 218.3.

A two-necked flask is charged with 2-(methylbicyclo[2.2.1]heptan-2-yl)methane methanesulfonate (3.16 g, 1 Eq, 14.5 mmol) and sodium iodide (4.77 g, 2.2 Eq, 31.8 mmol) in dry DMF (72 mL) and the resulting mixture is stirred at room temperature for 20 min. Then sodium cyanide (1.42 g, 2 Eq, 29 mmol) is added and the resulting reaction is stirred at 80° C. for 5 days. After reaction completion, the mixture is allowed to cool down to room temperature and then quenched with brine. The mixture is diluted with diethyl ether and the layers are separated. The aqueous layer is extracted six more times with diethyl ether. The combined organics are washed with brine, dried over Na₂SO₄, filtered and evaporated under reduced pressure. The crude residue is purified by flash column chromatography (SiO₂, 0-40% diethyl ether in heptane) affording 2-(methylbicyclo[2.2.1]heptan-2-yl)acetonitrile (686 mg, 4.3 mmol, 30% yield) as a colorless oil (74:26 mixture of isomers based on GCMS). GC-MS (Method 1): EI⁺, m/z: 149.1. ¹H NMR (400 MHz, CDCl3) δ 2.35 (d, J=16.5 Hz, 1H), 2.29 (d, J=13.6 Hz, 1H), 2.25 (d, J=6.4 Hz, 1H), 2.04 (d, J=3.5 Hz, 1H), 1.66 (dp, J=10.2, 2.0 Hz, 1H), 1.63-1.56 (m, 1H), 1.53-1.47 (m, 1H), 1.44 (ddd, J=12.9, 4.6, 2.8 Hz, 2H), 1.29-1.24 (m, 1H), 1.17 (s, 3H), 1.11-1.04 (m, 1H), 1.02 (dd, J=12.4, 2.8 Hz, 1H).

### Example 66: 2-(Methylbicyclo[2.2.1]hept-5-en-2-yl)acetonitrile

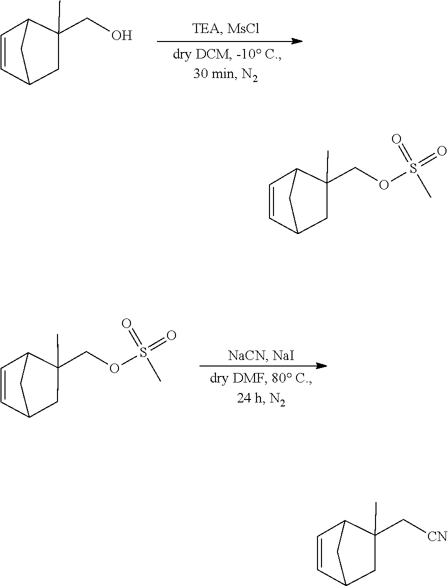

To a solution of 2-(methylbicyclo[2.2.1]hept-5-en-2-yl)methanol (1.5 g, 82% Wt., 1 Eq, 8.9 mmol) (Example 63) in DCM (36 mL), TEA (2.0 mL, 1.6 Eq, 14 mmol) is added under nitrogen atmosphere and the reaction mixture is then cooled down to −10° C. To the cold solution, methanesulfonyl chloride (0.83 mL, 1.2 Eq, 11 mmol) is slowly added dropwise via syringe over a period of 5 min. After addition, the mixture is left stirring at room temperature for 30 min. The reaction is then stopped and slowly quenched with ice/brine 1:1 solution. The layers are separated, and the aqueous layer is twice more extracted with DCM. The combined organics are washed with brine, dried over Na₂SO₄, filtered and evaporated under reduced pressure, affording a crude brown oil. The crude oil is purified by flash column chromatography (SiO₂, 0-40% diethyl ether in heptane) affording 2-(methylbicyclo[2.2.1]hept-5-en-2-yl)methyl methanesulfonate (1.0 g, 4.6 mmol, 55% yield) as a colorless oil (60:40 mixture of isomers). GC-MS (Method 1): EI⁺, m/z: 120.1 [fragment mass], calcd 216.1. LCMS (Method 2): M+H=121.1 [fragment mass]; calcd. 216.086.

A two-necked flask is charged with 2-(methylbicyclo[2.2.1]hept-5-en-2-yl)methane methanesulfonate (1.0 g, 1 Eq, 4.6 mmol) and sodium iodide (1.5 g, 2.2 Eq, 10 mmol) in dry DMF (23 mL) and the resulting mixture is stirred at room temperature for 20 min. Then sodium cyanide (0.45 g, 2 Eq, 9.2 mmol) is added and the resulting reaction is stirred at 80° C. for 24 h. After reaction completion, the mixture is allowed to cool down to room temperature and then quenched with brine. The mixture is diluted with diethyl ether and layers are separated. The aqueous layer is extracted three more times with diethyl ether. The combined organics are washed with brine, dried over Na₂SO₄, filtered and evaporated under reduced pressure. The crude residue is purified by flash column chromatography (SiO₂, 0-40% diethyl ether in heptane) affording the title compound (483 mg, 3.17 mmol, 69% yield) as a colorless oil (70:30 mixture of isomers). GCMS (Method 3): 147.2. ¹H NMR (400 MHz, CDCl₃) δ 6.16 (tdd, J=15.6, 5.7, 3.0 Hz, 2H), 2.86 (s, 1H, major), 2.58 (tt, J=2.8, 1.5 Hz, 1H, major), 2.52 (d, J=16.6 Hz, 1H, minor), 2.44 (d, J=16.6 Hz, 1H, minor), 2.19 (s, 1H, major), 1.66 (dt, J=9.2, 1.7 Hz, 1H), 1.60 (ddd, J=12.0, 3.7, 1.7 Hz, 1H, minor), 1.53-1.50 (m, 1H), 1.39 (s, 2H, major), 1.08 (s, 1H, minor), 1.01 (td, J=12.2, 2.4 Hz, 1H).

### Example 67: 2-(Bicyclo[2.2.1]heptan-2-yl)propanenitrile

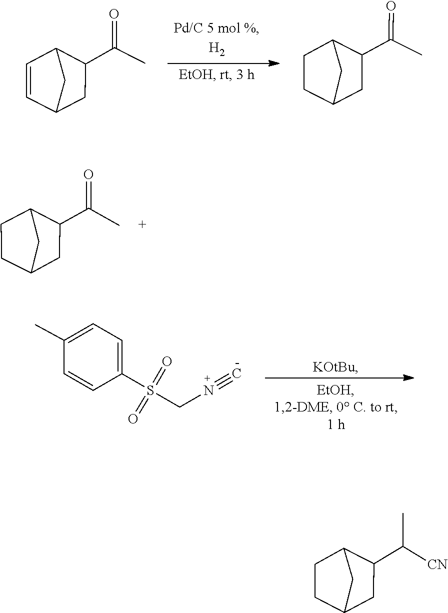

A stirred solution of 2-acetyl-5-norbornene (1.96 mL, 1 Eq, 14.7 mmol) in EtOH (73.4 mL) is purged with nitrogen for 5 min. Palladium, 10% on activated carbon, (1.56 g, 5% Wt., 0.05 Eq, 0.73 mmol) is added and the solution is purged with nitrogen for another 5 min. The atmosphere is replaced by hydrogen, by submitting the mixture to three cycles of vacuum/hydrogen. The mixture is stirred at room temperature for 3 h. The reacting mixture is filtered over celite, and the filter cake is washed with EtOH and EtOAc. The filtrate is evaporated under reduced pressure to give 2-acetyl norbornane (2.45 g, 12 mmol, 65% wt., 78% yield) as a colorless oil (1:1 mixture of isomers). GC-MS (Method 1): EI⁺, m/z: 138.1. ¹H NMR (400 MHz, CDCl3) δ 2.84 (dtd, J=11.0, 4.6, 2.0 Hz, 0.5H, minor), 2.62 (dq, J=4.1, 2.2 Hz, 0.5H, minor), 2.48-2.40 (m, 1H), 2.31-2.22 (m, 1H), 2.16 (s, 1.5H), 2.08 (s, 1.5H), 1.85 (dddd, J=12.4, 5.4, 4.2, 2.8 Hz, 0.5H, minor), 1.75 (ddd, J=12.3, 4.8, 2.5 Hz, 0.5H), 1.63-1.06 (m, 7H).

A solution of potassium tert-butoxide in THF (24.5 mL, 1.6 molar, 3.4 Eq, 39.2 mmol) is added dropwise to a stirred and solution of 2-acetyl-5-norbornane (2.45 g, 65% wt., 1 Eq, 11.5 mmol) and tosylmethyl isocyanide (4.05 g, 1.8 Eq, 20.7 mmol) in a mixture of 1,2-dimethoxyethane (57.6 mL) and ethanol (0.67 mL, 1 Eq, 11.5 mmol) at 0° C. After the addition, the mixture is allowed to reach room temperature and stirring for 1 h. The solution is diluted with water and extracted with diethyl ether, the layers are separated, and the aqueous layer is further extracted with diethyl ether twice. The organics are combined, dried over Na₂SO₄, filtered and concentrated under reduced pressure. The crude mixture is purified by flash column chromatography (SiO₂, 0-50% MTBE in heptane) to afford the title compound (759 mg, 5.08 mmol, 44% yield) as a colorless oil (mixture of isomers, ratio not determined). GC-MS (Method 1): EI⁺, m/z: 149.1. ¹H NMR (400 MHz, CDCl3) δ 2.51-2.09 (m, 3H), 2.02-1.74 (m, 0.5H), 1.69-1.43 (m, 3H), 1.43-1.10 (m, 7.5H), 1.09-0.61 (m, 1H).

### Examples 68-76: Synthetic Methods

The Compound of Examples 68-76 may be prepared using the aforementioned procedures in combination with standard chemical synthetic methods for ester synthesis. For example, the following general schemes may be followed:

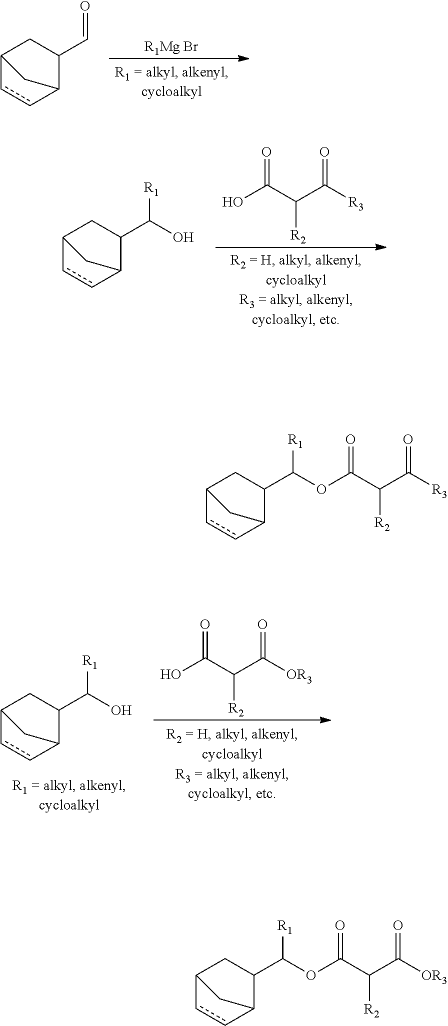

### Example 77: Odor Profile Determination

Odor profile for selected compounds within the scope of the disclosure is determined using assessments by one Master Perfumer and 4-6 trained sensory panelists. Sensory panelists are trained on our internal taxonomy with reference materials that include 11 grand families, and 62 subfamilies. Panelists are experienced in, but not trained in, an additional several hundred odor descriptors. Panelists are trained on usage of a rating scale, which also includes internal references. Panelists are not advanced to compound evaluation until they have completed and received a passing score on a Final Exam on the taxonomy that was curated by our perfumery team.

A single score is generated for the sensory panel by taking the arithmetic mean of all panelist scores for a given sample, for a given attribute. Alternatively, a single score may be generated by fitting all panelist scores to a model that can correct for differences in the intercept or slope of the latent function through which each panelist produces a rating given a percept, i.e. correction for inter-panelist variation.

The Master Perfumer, possessing a deep knowledge of fragrance ingredients, high level technical expertise, and who is recognized for their experience in creating complex perfumes, reviews, may augment sensory panel scores for improved accuracy.

Test samples are dissolved at a concentration of 10% w/v in ethanol. New test blotters (White Paper Paddle Shaped Perfumery Blotters, measuring 5×0.5 inches) are dipped into the 10% solution of the text compound. Odor descriptions are captured within 1-2 minutes of wetting the blotter to allow for evaporation of most of the ethanol solvent, permitting a more accurate determination of the test compound's odor. Odor intensity is ranked on a scale of 1 to 10. Assessments are made at ambient temperature in a benchtop laboratory setting.

The results are shown in the table below:

| | | Odor | Odor |
|---|---|---|---|
| Ex. No. | Structure | Description | Intensity |
| 1 | 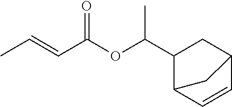 | sweet milky fruity melony ozonic metallic | 7 |
| 2 | 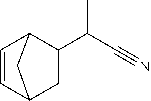 | melon leather fresh beautiful floral salicylate berries tropical salicylate | 6 |
| 3 | 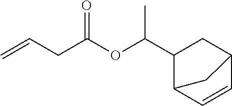 | melon herbal tarragon | 4 |
| 4 | 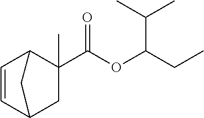 [SMILES: CCC(OC(=O)C1(C)CC2C=CC1C2)C(C)C] | sweet floral | 8 |
| 5 | 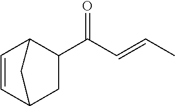 | metallic salty woody | 9 |
| 6 | 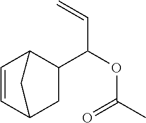 | woody camphoraceous woody | 9 |
| 7 | 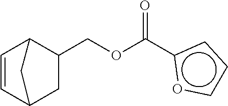 | earthy fatty | 7 |
| 8 | 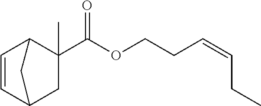 [SMILES: CC/C=C\CCOC(=O)C1(C)CC2C=CC1C2] | muguet grapefruity citrus blood- orange | 6.5 |
| 9 | 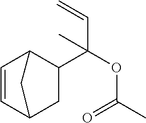 | fruity watery green lavender floral | 7 |
| 10 | 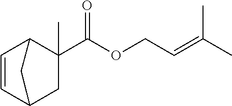 [SMILES: CC(C)=CCOC(=O)C1(C)CC2C=CC1C2] | pear apple fruity watery | 7 |
| 11 | 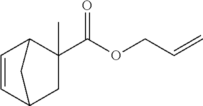 | woody smokey | 7 |
| 12 | 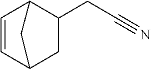 | starchy soulful | 7 |
| 13 | 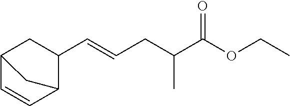 [SMILES: CCOC(=O)C(C)C/C=C/C1CC2C=CC1C2] | soulful chestnut nutty pumpkin | 6 |
| 14 | 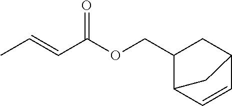 | green watery fruity peach pear mushroom | 7 |
| 15 | 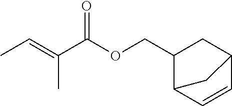 | fruity waxy pear rosy fatty oily aldehydic orange | 7 |
| 16 | 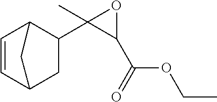 [SMILES: CCOC(=O)C1OC1(C)C1CC2C=CC1C2] | strawberry sweet green caramel fresh | 6.5 |
| 17 | 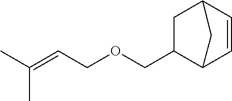 | hay tobacco peach fruity | 5 |
| 18 | 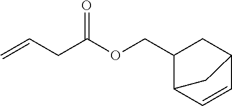 | metallic nutty earthy mushroom | 6 |
| 19 | 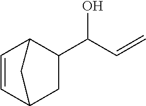 | medicinal camphoraceous watery earthy fruity green piney | 6 |
| 20 | 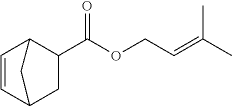 | green hay watery | 6 |
| 21 | 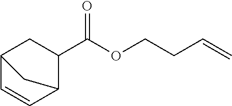 | apricot pineapple | 6 |
| 22 | 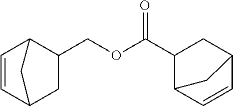 [SMILES: O=C(OCC1CC2C=CC1C2)C1CC2C=CC1C2] | sweet chestnut flesh rummy fruit-cake | 6 |
| 23 | 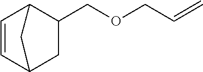 | chemical, green | 9 |
| 24 | 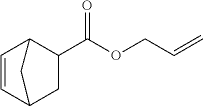 | wet fruity | 9 |
| 25 | 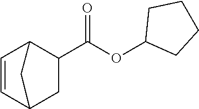 [SMILES: O=C(OC1CCCC1)C1CC2C=CC1C2] | amber sweet spicy melon raspberry | 7 |
| 26 | 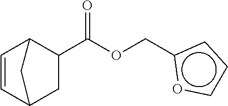 | apple pear melon fruity | 7 |
| 27 | 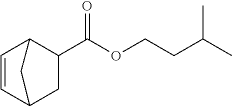 | melon sweet powdery sweet grainy pear watery | 6 |
| 28 | 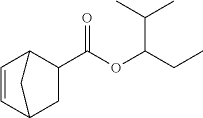 [SMILES: CCC(OC(=O)C1CC2C=CC1C2)C(C)C] | woody watery green fruity | 8 |
| 29 | 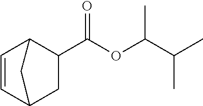 | rosey powdery | 7 |
| 30 | 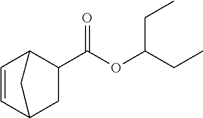 | creamy berry woody peachy | 7 |
| 31 | 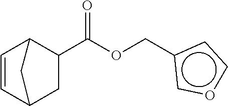 | floral metallic | 6 |
| 32 | 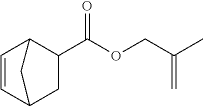 | bell peppery sl.-metallic | 9 |
| 33 | 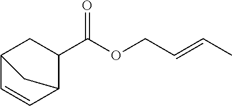 | woody apricot mango fruity | 7 |
| 34 | 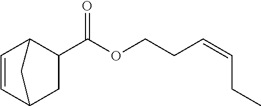 [SMILES: CC/C=C\CCOC(=O)C1CC2C=CC1C2] | green | 6 |
| 35 | 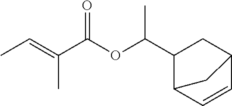 [SMILES: C/C=C(\C)C(=O)OC(C)C1CC2C=CC1C2] | waxy fruity butterscotch caramel | 6 |
| 36 | 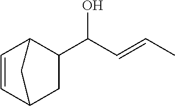 | fruity vanilla smoky leathery | 9 |

Additional compounds are tested as described above, but with the odor evaluation being captured at four time points: t=1-2 minutes, about 30 minutes, about 5 hours, and about 24 hours. Odor intensity is ranked on a scale of 1 to 10. Assessments are made at ambient temperature in a benchtop laboratory setting, and the blotters are left out on the benchtop after blotting. The results are shown below:

| | | | Odor Intensity | | | |
|---|---|---|---|---|---|---|
| Ex. | | Odor | 1-2 | 30 | | |
| No. | Structure | Description | min | min | 5 hr | 24 hr |
| 13A | 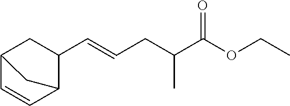 [SMILES: CCOC(=O)C(C)C/C=C/C1CC2C=CC1C2] | soulful, nutty, starchy, baked pastry, woody, chestnut, pumpkin | 6 | 6 | 4.5 | 4 |
| 37 | 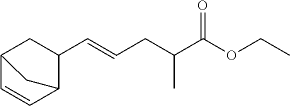 [SMILES: CCOC(=O)C(C)C/C=C/C1CC2C=CC1C2] | fruity, mineral, animalic, soulful, green, fermented, starchy | 6 | 7.5 | 6.5 | 6.5 |
| 38 | 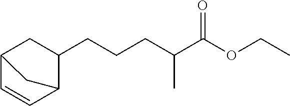 [SMILES: CCOC(=O)C(C)CCCC1CC2C=CC1C2] | fruity, floral, green, tropical | 4.25 | 6 | 1 | 0 |
| 39 | 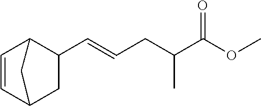 [SMILES: COC(=O)C(C)C/C=C/C1CC2C=CC1C2] | fruity, mineral, citrus, floral, soulful, oily, wet, peary, tropical | 6.5 | 7 | 5 | 4.5 |
| 40 | 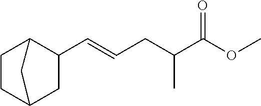 [SMILES: COC(=O)C(C)C/C=C/C1CC2CCC1C2] | fruity, soulful, green, oily, starchy | 6.5 | 6 | 6 | 6 |
| 41 | 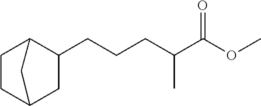 [SMILES: COC(=O)C(C)CCCC1CC2CCC1C2] | fruity, woody, animalic, green, tropical | 6 | 6.5 | 4 | 4 |
| 42 | 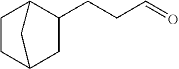 [SMILES: O=CCCC1CC2CCC1C2] | floral, muguet, fruity, green, overripe | 8 | 8 | 4 | 3.5 |
| 43 | 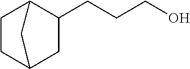 [SMILES: OCCCC1CC2CCC1C2] | floral, muguet, clean, rose, fresh | 4 | 6 | 2.5 | 2 |
| 44 | 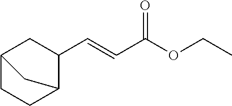 | fruity, floral, green, tropical, cedarwood | 6 | 6 | 4 | 3 |
| 45 | 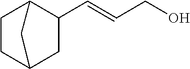 [SMILES: OC/C=C/C1CC2CCC1C2] | fruity, soulful, green, starchy, grassy, peary, | 5 | 6 | 3 | 3 |
| | | peachy, rosy, | | | | |
| 46 | 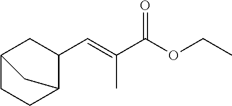 | floral, fresh, woody, fruity, green, herbal, sweet | 5 | 5 | 2 | 2 |
| 47 | 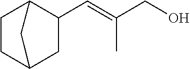 | citrus, floral, woody, green, limey, aldehydic, stemmy, | 5 | 6 | 6 | 5.5 |
| | | herbal, | | | | |
| | | muguet | | | | |
| 48 | 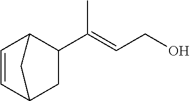 | green, soulful, cucumbery, violet leaf | 6.5 | 7.5 | 6.5 | 6.5 |
| 49 | 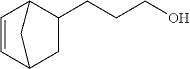 [SMILES: OCCCC1CC2C=CC1C2] | fruity, mineral, citrus, floral, green, tropical, wet, | 6 | 6 | 6 | 6 |
| | | violetty, | | | | |
| | | metallic | | | | |
| 50 | 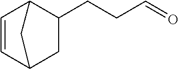 [SMILES: O=CCCC1CC2C=CC1C2] | green, floral, cucumbery, violey leaf, spicy | 7.5 | 7 | 5 | 3 |
| 51 | 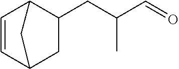 | melony, honey, starchy, aldehydic, fruity, floral, | 7 | 7 | 4 | 4 |
| | | woody, | | | | |
| | | herbal | | | | |
| 52 | 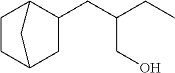 | floral, green, muguet, powdery, violetty, gourmand | 3.25 | 3 | 2 | 0 |
| 53 | 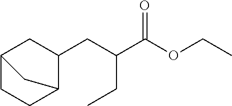 [SMILES: CCOC(=O)C(CC)CC1CC2CCC1C2] | fruity, appley, peary, grassy, banana, fresh, muguet, woody, clean | 6 | 6 | 3.5 | 2.5 |
| 54 | 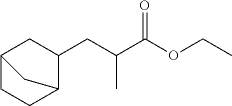 | fruity, appley, peary, driftwoody, creamy, apricot, woody, vetiver | 5 | 4 | 2.5 | 2 |
| 55 | 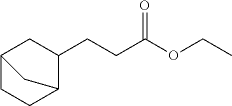 | fruity, appley, peary, mineral, wet | 6 | 6 | 2 | 2 |
| 56 | 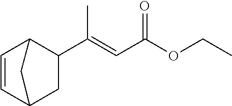 | floral, fruity, green, herbal, rose | 4 | 5 | 3 | 2 |
| 57 | 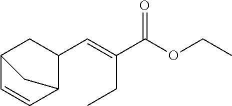 [SMILES: CCOC(=O)/C(=C/C1CC2C=CC1C2)CC] | floral, green, fruity, fresh, jasmin, herbal | 5 | 6 | 4 | 2 |
| 58 | 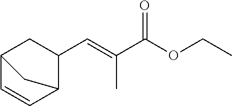 | floral, green, fruity, fresh, jasmin | 5 | 5 | 4 | 3 |
| 59 | 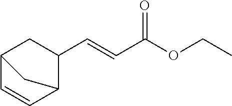 | fruity, green, floral, sweet, melon, tropical | 5 | 5 | 3 | 3 |
| 60 | 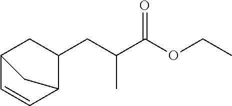 | fruity, floral, green, jasmin, rose, sweet | 5 | 4 | 2.5 | 2 |
| 61 | 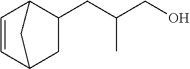 | tropical, green, earthy, cedarwood, muguet | 6 | 4 | 4 | 4 |
| 62 | 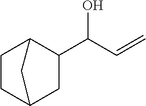 | herbal, green, floral, woody, fresh, miny | 6 | TBD | TBD | TBD |
| 63 | 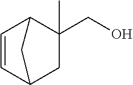 | TBD | TBD | TBD | TBD | TBD |
| 64 | 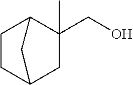 | TBD | TBD | TBD | TBD | TBD |
| 65 | 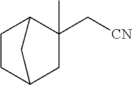 | TBD | TBD | TBD | TBD | TBD |
| 66 | 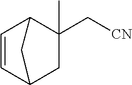 | TBD | TBD | TBD | TBD | TBD |
| 67 | 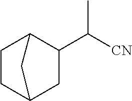 | TBD | TBD | TBD | TBD | TBD |
| 68 | 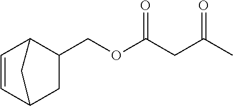 | TBD | TBD | TBD | TBD | TBD |
| 69 | 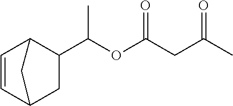 [SMILES: CC(=O)CC(=O)OC(C)C1CC2C=CC1C2] | TBD | TBD | TBD | TBD | TBD |
| 70 | 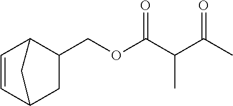 [SMILES: CC(=O)C(C)C(=O)OCC1CC2C=CC1C2] | TBD | TBD | TBD | TBD | TBD |
| 71 | 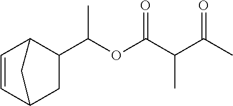 [SMILES: CC(=O)C(C)C(=O)OC(C)C1CC2C=CC1C2] | TBD | TBD | TBD | TBD | TBD |
| 72 | 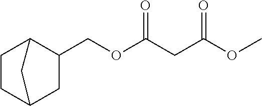 [SMILES: COC(=O)CC(=O)OCC1CC2CCC1C2] | TBD | TBD | TBD | TBD | TBD |
| 73 | 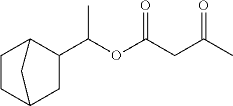 [SMILES: CC(=O)CC(=O)OC(C)C1CC2CCC1C2] | TBD | TBD | TBD | TBD | TBD |
| 74 | 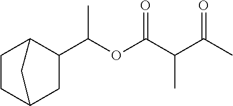 [SMILES: CC(=O)C(C)C(=O)OC(C)C1CC2CCC1C2] | TBD | TBD | TBD | TBD | TBD |
| 75 | 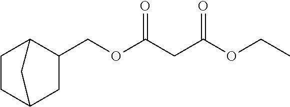 [SMILES: CCOC(=O)CC(=O)OCC1CC2CCC1C2] | TBD | TBD | TBD | TBD | TBD |
| 76 | 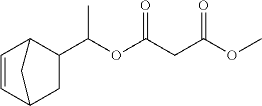 [SMILES: COC(=O)CC(=O)OC(C)C1CC2C=CC1C2] | TBD | TBD | TBD | TBD | TBD |

The Examples provided herein are exemplary only and are not intended to be limiting in any way to the various aspects and embodiments of the invention described herein.

What is claimed:

1. A compound of Formula I or Formula II: 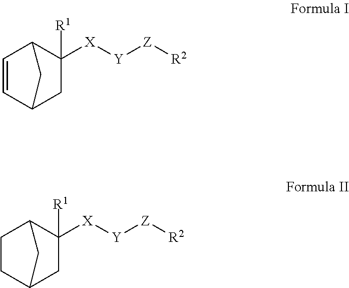
R¹ is H, C₁₋₅alkyl, C₃₋₅cycloalkyl, OC₁₋₅alkyl, or OC₃₋₅cycloalkyl;
X is selected from O, C(R^{X1})(R^{X2}), C(O), C(O)O, C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Y is selected from a bond, C(R^{X1})(R^{X2}), C(O), C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Z is selected from a bond, O, CN, CHO, C(O)O, C(O), C(R^{X1})(R^{X2})O, C(R^{X1})(R^{X2})CN, C(R^{X1})(R^{X2})CHO, C(R^{X1})(R^{X2})C(O), C(R^{X1})(R^{X2})OC(O), and C(R^{X1})(R^{X2})C(O)O, provided that when Z is CN, CHO, C(R^{X1})(R^{X2})CN, or C(R^{X1})(R^{X2})CHO, then R² is absent;
or wherein X—Y—Z—R² is: 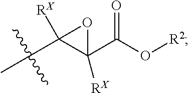
R² is H, OH, C₁₋₆alkyl, C₂₋₆alkenyl, C₃₋₆cycloalkyl, C₃₋₆cycloalkenyl, C₆₋₁₂bicycloalkyl, C₆₋₁₂bicycloalkenyl, aryl, or heteroaryl, or when Z is CN, CHO, C(R^{X1})(R^{X2})CN, or C(R^{X1})(R^{X2})CHO, then R² is absent;
R^{X} is selected from H, C₁₋₆alkyl, C₂₋₆alkenyl, and C₃₋₆cycloalkyl;
R^{X1} and R^{X2} are each independently selected from H, OH, C₁₋₆alkyl, C₂₋₆alkenyl, and C₃₋₆cycloalkyl;
provided that the group X—Y—Z— does not form a peroxide (—O—O—) or acid anhydride ((C(O)—O—C(O)) structure; and
optionally provided that Y is not C(O), C(R^{X1})(R^{X2})O, or C(R^{X1})(R^{X2})C(O), when Z is O, CN, CHO, C(O)O, or C(O);
and all enantiomers, diastereomers, endo/exo, and E/Z isomers thereof;
in free or salt form;
provided that the compound is not any of the Selected Compounds.

2. A compound of Formula I or Formula II: 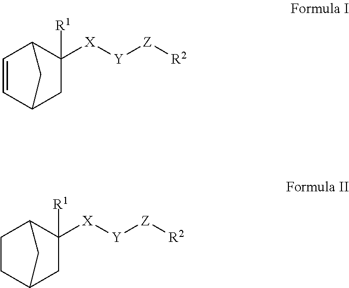
R¹ is H, C₁₋₅alkyl, C₃₋₅cycloalkyl, OC₁₋₅alkyl, or OC₃₋₅cycloalkyl;
X is selected from O, C(R^{X1})(R^{X2}), C(O), C(O)O, C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Y is selected from a bond, C(R^{X1})(R^{X2}), C(O), C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Z is selected from a bond, O, CN, CHO, C(O)O, C(O), C(R^{X1})(R^{X2})O, C(R^{X1})(R^{X2})CN, C(R^{X1})(R^{X2})CHO, C(R^{X1})(R^{X2})C(O), C(R^{X1})(R^{X2})OC(O), and C(R^{X1})(R^{X2})C(O)O, provided that when Z is CN, CHO, C(R^{X1})(R^{X2})CN, or C(R^{X1})(R^{X2})CHO, then R² is absent;
or wherein X—Y—Z—R² is: 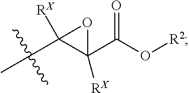
R² is H, OH, C₁₋₆alkyl, C₂₋₆alkenyl, C₃₋₆cycloalkyl, C₃₋₆cycloalkenyl, C₆₋₁₂bicycloalkyl, C₆₋₁₂bicycloalkenyl, aryl, or heteroaryl, or when Z is CN, CHO, C(R^{X1})(R^{X2})CN, or C(R^{X1})(R^{X2})CHO, then R² is absent;
R^{X} is selected from H, C₁₋₆alkyl, C₂₋₆alkenyl, and C₃₋₆cycloalkyl;
R^{X1} and R^{Xa} are each independently selected from H, OH, C₁₋₆alkyl, C₂₋₆alkenyl, and C₃₋₆cycloalkyl;
provided that the group X—Y—Z— does not form a peroxide (—O—O—) or acid anhydride ((C(O)—O—C(O)) structure; and
optionally provided that Y is not C(O), C(R^{X1})(R^{X2})O, or C(R^{X1})(R^{X2})C(O), when Z is O, CN, CHO, C(O)O, or C(O);
and all enantiomers, diastereomers, endo/exo, and E/Z isomers thereof;
in free or salt form;
optionally provided that the compound is not one or more of the Selected Compounds;
for use as a flavor or fragrance ingredient.

3. A flavor composition and/or fragrance composition comprising a compound of Formula I or Formula II: 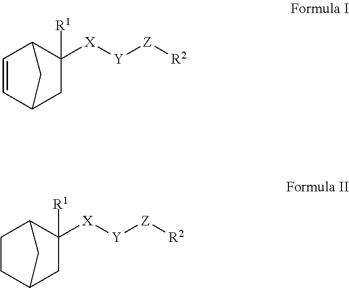
R¹ is H, C₁₋₅alkyl, C₃₋₅cycloalkyl, OC₁₋₅alkyl, or OC₃₋₅cycloalkyl;
X is selected from O, C(R^{X1})(R^{X2}), C(O), C(O)O, C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Y is selected from a bond, C(R^{X1})(R^{X2}), C(O), C(R^{X1})(R^{X2})C(R^{X1})(R^{X2}), C(R^{X})═C(R^{X}), C(R^{X1})(R^{X2})O, OC(R^{X1})(R^{X2}), C(R^{X1})(R^{X2})C(O), and C(O)C(R^{X1})(R^{X2});
Z is selected from a bond, O, CN, CHO, C(O)O, C(O), C(R^{X1})(R^{X2})O, C(R^{X1})(R^{X2})CN, C(R^{X1})(R^{X2})CHO, C(R^{X1})(R^{X2})C(O), C(R^{X1})(R^{X2})OC(O), and C(R^{X1})(R^{X2})C(O)O, provided that when Z is CN, CHO, C(R^{X1})(R^{X2})CN, or C(R^{X1})(R^{X2})CHO, then R² is absent;
or wherein X—Y—Z—R² is: 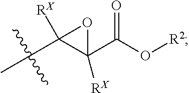
R² is H, OH, C₁₋₆alkyl, C₂₋₆alkenyl, C₃₋₆cycloalkyl, C₃₋₆cycloalkenyl, C₆₋₁₂bicycloalkyl, C₆₋₁₂bicycloalkenyl, aryl, or heteroaryl, or when Z is CN, CHO, C(R^{X1})(R^{X2})CN, or C(R^{X1})(R^{X2})CHO, then R² is absent;
R^{X} is selected from H, C₁₋₆alkyl, C₂₋₆alkenyl, and C₃₋₆cycloalkyl;
R^{X1} and R^{X2} are each independently selected from H, OH, C₁₋₆alkyl, C₂₋₆alkenyl, and C₃₋₆cycloalkyl;
provided that the group X—Y—Z— does not form a peroxide (—O—O—) or acid anhydride ((C(O)—O—C(O)) structure; and
optionally provided that Y is not C(O), C(R^{X1})(R^{X2})O, or C(R^{X1})(R^{X2})C(O), when Z is O, CN, CHO, C(O)O, or C(O);
and all enantiomers, diastereomers, endo/exo, and E/Z isomers thereof;
in free or salt form;
optionally provided that the compound is not one or more of the Selected Compounds;
in admixture with one or more non-toxic, orally acceptable, pharmaceutically acceptable, cosmetically acceptable, or acceptable for a household product, carriers or excipients.

4. The composition of claim 3, wherein the compound of Formula I is one or more of the Selected Compounds.

5. The composition of claim 3, wherein the compound is a Compound of Formula Ia or IIa: 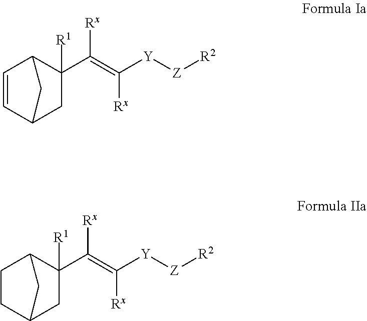
wherein Y is C(R^{X1})(R^{X2})C(R^{X1})(R^{X2}) or a bond, Z is CHO, C(O)O, C(O), C(R^{X1})(R^{X2})O, or C(R^{X1})(R^{X2})OC(O), and R² is H, C₁₋₅alkyl, C₃₋₅cycloalkyl, or C₂₋₅alkenyl.

6. The composition of claim 3, wherein the compound is a Compound of Formula Ib or IIb: 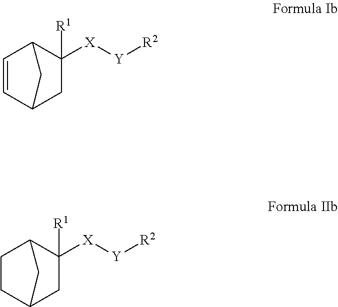
wherein X is C(R^{X1})(R^{X2})O, or C(O)(O), and Y is C(R^{X1})(R^{X2}) or C(O), provided that X is not C(O)O when Y is C(O).

7. The composition of claim 3, wherein the compound is a Compound of Formula Ic or IIc: 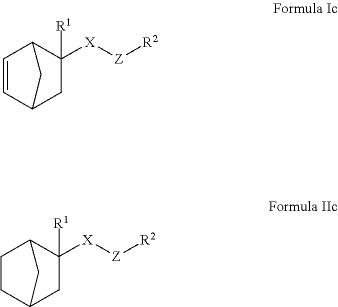
wherein X is C(R^{X1})(R^{X2}) or C(R^{X1})(R^{X2})C(R^{X1})(R^{X2}), and Z is CN, CHO, C(R^{X1})(R^{X2})CN, C(R^{X1})(R^{X2})CHO, C(R^{X1})(R^{X2})O, and C(R^{X1})(R^{X2})C(O)O

8. The composition of claim 3, wherein the compound is a Compound of Formula Id or IId: 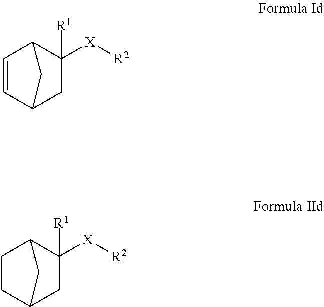
wherein X is C(R^{X1})(R^{X2}), C(R^{X1})(R^{X2})O, C(O) or C(O)(O), or
wherein X—R² is:

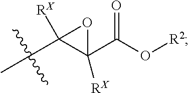

9. The composition of claim 3, wherein the compound is a Compound of Formula Ie or IIe: 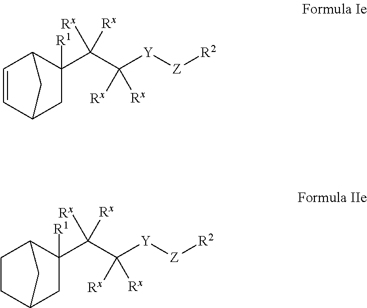
wherein R^{X} is independently selected from H, C₁₋₆alkyl, C₂₋₆alkenyl, and C₃₋₆cycloalkyl, Y is C(R^{X1})(R^{X2})C(R^{X1})(R^{X2}) or a bond, Z is CHO, C(O)O, C(O), C(R^{X1})(R^{X2})O, or C(R^{X1})(R^{X2})OC(O), and R² is H, C₁₋₅alkyl, C₃₋₅cycloalkyl, or C₂₋₅alkenyl;

10. The composition of claim 3, wherein the compound is a Compound of Formula If or IIf: 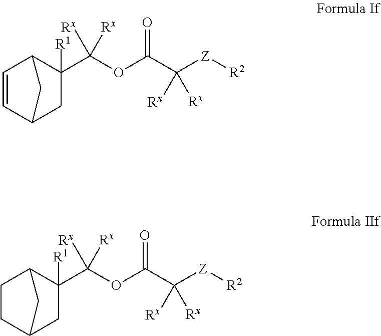
wherein R^{X} is independently selected from H, C₁₋₆alkyl, C₂₋₆alkenyl, and C₃₋₆cycloalkyl, Z is CN, CHO, C(O)O, or C(O), and R² is H, C₁₋₅alkyl, C₃₋₅cycloalkyl, or C₂₋₅alkenyl;
provided that when Z is CN or CHO, then R² is absent.

11. The composition of claim 3, wherein the composition further comprises one or more solvents.

12. The composition of claim 3, wherein the composition further comprises one or more other flavors or fragrances.

13. A product comprising the composition of claim 3, e.g., a product selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.
